SEQUENCE LISTING (68 sequences)

SEQ ID NO: 1 - protein, 10 aa, Artificial Sequence
  source: /note=“Description of Artificial Sequence Synthetic peptide”
GYTFTSYNMH

SEQ ID NO: 2 - protein, 10 aa, Artificial Sequence
  source: /note=“Description of Artificial Sequence Synthetic peptide”
AIYPGNGDTS

SEQ ID NO: 3 - protein, 12 aa, Artificial Sequence
  source: /note=“Description of Artificial Sequence Synthetic peptide”
STYYGGDWYFNV

SEQ ID NO: 4 - protein, 10 aa, Artificial Sequence
  source: /note=“Description of Artificial Sequence Synthetic peptide”
RASSSVSYIH

SEQ ID NO: 5 - protein, 7 aa, Artificial Sequence
  source: /note=“Description of Artificial Sequence Synthetic peptide”
ATSNLAS

SEQ ID NO: 6 - protein, 9 aa, Artificial Sequence
  source: /note=“Description of Artificial Sequence Synthetic peptide”
QQWTSNPPT

SEQ ID NO: 7 - protein, 10 aa, Artificial Sequence
  source: /note=“Description of Artificial Sequence Synthetic peptide”
GYTFSSYNMH

SEQ ID NO: 8 - protein, 10 aa, Artificial Sequence
  source: /note=“Description of Artificial Sequence Synthetic peptide”
AIYPGAGDTS

SEQ ID NO: 9 - protein, 13 aa, Artificial Sequence
  source: /note=“Description of Artificial Sequence Synthetic peptide”
SNYYGSSGWYFDV

SEQ ID NO: 10 - protein, 10 aa, Artificial Sequence
  source: /note=“Description of Artificial Sequence Synthetic peptide”
RASLSVSSMH

SEQ ID NO: 11 - protein, 9 aa, Artificial Sequence
  source: /note=“Description of Artificial Sequence Synthetic peptide”
QQWIFNPPT

SEQ ID NO: 12 - protein, 121 aa, Artificial Sequence
  source: /note=“Description of Artificial Sequence Synthetic polypeptide”
QVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 13 - DNA, 363 bp, Artificial Sequence
  source: /note=“Description of Artificial Sequence Synthetic polynucleotide”
caggttcagctggttcagtctggtgccgaagtgaagaaacctggctcctccgtgaaagtgtcctgcaaggcttccggctacacttttaccagctacaacatgcactgggtccgacaggcccctggacaaggattggaatggatgggcgctatctaccccggcaacggcgatacctcttacgcccagaaattccagggcagagtgaccatcaccgccgacaagtctacctccaccgcctacatggaactgtccagcctgagatctgaggacaccgccgtgtactactgcgcccggtctacctattatggcggcgactggtacttcaacgtgtggggccagggaaccctggtcacagtctcttct

SEQ ID NO: 14 - protein, 106 aa, Artificial Sequence
  source: /note=“Description of Artificial Sequence Synthetic polypeptide”
AIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIK

SEQ ID NO: 15 - DNA, 318 bp, Artificial Sequence
  source: /note=“Description of Artificial Sequence Synthetic polynucleotide”
gccattcagctgacccagtctccatcctctctgtccgcctctgtgggcgacagagtgacaattacctgccgggcctcctcctccgtgtcctacatccattggttccagcagaagcccggcaaggcccctaagcctctgatctacgccacctccaatctggcctctggcgtgccctccagattttccggatctggctctggaaccgactttaccctgacaatctccagcctgcagcctgaggacttcgccacctactactgtcagcagtggaccagcaatcctcctacctttggccagggcaccaagctggaaatcaag

SEQ ID NO: 16 - protein, 122 aa, Artificial Sequence
  source: /note=“Description of Artificial Sequence Synthetic polypeptide”
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSS

SEQ ID NO: 17 - DNA, 366 bp, Artificial Sequence
  source: /note=“Description of Artificial Sequence Synthetic polynucleotide”
caggttcaactggttcagtctggcgccgaagtgaagaaacctggctcctccgtgaaggtgtcctgcaaggcttccggctacaccttctccagctacaacatgcactgggtccgacaggcccctggacaaggattggaatggatgggcgctatctaccctggcgctggcgatacctcttacgcccagaaattccagggcagagtgaccatcaccgccgacgagtctacctccaccgcctacatggaactgtccagcctgagatctgaggacaccgccgtgtactactgcgcccggtctaattactacggctccagcggctggtacttcgacgtgtggggaaagggcaccaccgtgacagtctcttct

SEQ ID NO: 18 - protein, 106 aa, Artificial Sequence
  source: /note=“Description of Artificial Sequence Synthetic polypeptide”
EIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIK

SEQ ID NO: 19 - DNA, 318 bp, Artificial Sequence
  source: /note=“Description of Artificial Sequence Synthetic polynucleotide”
gagatcgtgctgacccagtctccagccacactgtcactgtctccaggcgagagagctaccctgtcctgtagagcctctctgtccgtgtcctccatgcactggtatcagcagaagcctggacaggcccctcggctgctgatctacgctacctctaatctggccagcggtatccccgccagattttctggttctggctctggcaccgactttaccctgaccatctccagcctggaacctgaggacttcgccgtgtactactgccagcagtggatcttcaaccctcctacctttggcggaggcaccaaggtggaaatcaag

SEQ ID NO: 20 - protein, 121 aa, Artificial Sequence
  source: /note=“Description of Artificial Sequence Synthetic polypeptide”
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVYYGSNYWYFDVWGTGTTVTVSS

SEQ ID NO: 21 - DNA, 363 bp, Artificial Sequence
  source: /note=“Description of Artificial Sequence Synthetic polynucleotide”
caagcatatctgcaacagagcggagctgagctggttcggcctggcgcctctgtaaaaatgagttgcaaggccagtggttatacattcacatcatataatatgcactgggtaaagcaaactccccgacaggggcttgaatggattggcgcaatctatcccggcaatggggatacatcctacaatcagaaattcaagggcaaggcaacactgaccgttgacaaatcctcatcaacagcctacatgcagctcagttccctcactagcgaagattctgctgtgtatttctgtgcaagggtgtattatggttctaattactggtatttcgatgtttggggaaccggaactaccgtaactgtttctagc

SEQ ID NO: 22 - protein, 106 aa, Artificial Sequence
  source: /note=“Description of Artificial Sequence Synthetic polypeptide”
QIVLSQSPAILSASPGEKVTMTCRASSSVSYMHWYQQKPGSSPQVWIYATSNLASGVPVRFSGSGSGTSYSLTISRVEAEDTATYYCQQWIFNPPTFGSGTKLEIR

SEQ ID NO: 23 - DNA, 318 bp, Artificial Sequence
  source: /note=“Description of Artificial Sequence Synthetic polynucleotide”
caaatagtcctttcacagtccccagctattctttcagcctctcccggtgaaaaggttacaatgacctgccgggcaagctccagtgtctcatatatgcactggtaccaacaaaaacctggcagtagtcctcaggtgtggatctacgctacaagcaatctcgcttccggggttcccgtgaggtttagcggaagcgggtctggaactagttattccttgacaattagtcgggttgaagccgaggacaccgccacttactattgccaacagtggatattcaatccacccaccttcggttcaggtaccaagctcgaaatccgt

SEQ ID NO: 24 - protein, 122 aa, Artificial Sequence
  source: /note=“Description of Artificial Sequence Synthetic polypeptide”
QAYLQQSGAELVRPGASVKMSCKTSGYTFSSYNMHWVKQTPRQALEWIGAIYPGNGDTSYNQKFKGKATLTVDKSSSTAYMQLSSLTSEDSAVYFCTRSNYYGSSGWYFDVWGTGTTVTVSS

SEQ ID NO: 25 - DNA, 366 bp, Artificial Sequence
  source: /note=“Description of Artificial Sequence Synthetic polynucleotide”
caagcctatcttcaacaatctggggctgagcttgtccggccaggagcctccgtcaaaatgagctgcaaaacctcaggttatacttttagtagctataacatgcattgggtaaaacaaaccccccgacaagcattggagtggataggggccatataccccggcaatggagacacaagttacaaccagaagtttaaaggcaaagctacactcacagttgacaaatcctcaagtactgcttatatgcaactctcctctctcacttccgaagacagtgccgtatatttttgcactcggtccaattactatggatctagtggctggtactttgacgtttggggcactgggacaactgttacagtgtccagc

SEQ ID NO: 26 - protein, 106 aa, Artificial Sequence
  source: /note=“Description of Artificial Sequence Synthetic polypeptide”
QIVLSQSPAILSASPGEKVTMTCRASLSVSSMHWYQQKPGSSPKPWIYATSNLASGVPARFSGSGSGTSYSLTISRVEAEDAATYYCQQWIFNPPTFGGGTKLEIK

SEQ ID NO: 27 - DNA, 318 bp, Artificial Sequence
  source: /note=“Description of Artificial Sequence Synthetic polynucleotide”
cagattgtcctgagccaatccccagcaattctgagtgctagccctggagagaaggtaacaatgacttgtcgggcatcccttagcgtctcatccatgcattggtatcaacaaaagccaggttcatctccaaaaccctggatttacgctacatctaacctggcatctggggtgcctgccagatttagtggatctggttccggcacatcatattcccttacaatcagccgagtggaagccgaggatgctgcaacctattactgtcaacaatggatatttaaccctcccacctttgggggtgggactaaactcgaaatcaag

SEQ ID NO: 28 - protein, 20 aa, Artificial Sequence
  source: /note=“Description of Artificial Sequence Synthetic peptide”
GGSEGKSSGSGSESKSTGGS

SEQ ID NO: 29 - protein, 8 aa, Artificial Sequence
  source: /note=“Description of Artificial Sequence Synthetic peptide”
GGGSGGGS

SEQ ID NO: 30 - protein, 12 aa, Artificial Sequence
  source: /note=“Description of Artificial Sequence Synthetic peptide”
GGGSGGGSGGGS

SEQ ID NO: 31 - protein, 16 aa, Artificial Sequence
  source: /note=“Description of Artificial Sequence Synthetic peptide”
GGGSGGGSGGGSGGGS

SEQ ID NO: 32 - protein, 20 aa, Artificial Sequence
  source: /note=“Description of Artificial Sequence Synthetic peptide”
GGGSGGGSGGGSGGGSGGGS

SEQ ID NO: 33 - protein, 15 aa, Artificial Sequence
  source: /note=“Description of Artificial Sequence Synthetic peptide”
GGGGSGGGGSGGGGS

SEQ ID NO: 34 - protein, 20 aa, Artificial Sequence
  source: /note=“Description of Artificial Sequence Synthetic peptide”
GGGGSGGGGSGGGGSGGGGS

SEQ ID NO: 35 - protein, 25 aa, Artificial Sequence
  source: /note=“Description of Artificial Sequence Synthetic peptide”
GGGGSGGGGSGGGGSGGGGSGGGGS

SEQ ID NO: 36 - protein, 18 aa, Artificial Sequence
  source: /note=“Description of Artificial Sequence Synthetic peptide”
GSTSGSGKPGSGEGSTKG

SEQ ID NO: 37 - protein, 14 aa, Artificial Sequence
  source: /note=“Description of Artificial Sequence Synthetic peptide”
IRPRAIGGSKPRVA

SEQ ID NO: 38 - protein, 15 aa, Artificial Sequence
  source: /note=“Description of Artificial Sequence Synthetic peptide”
GKGGSGKGGSGKGGS

SEQ ID NO: 39 - protein, 15 aa, Artificial Sequence
  source: /note=“Description of Artificial Sequence Synthetic peptide”
GGKGSGGKGSGGKGS

SEQ ID NO: 40 - protein, 15 aa, Artificial Sequence
  source: /note=“Description of Artificial Sequence Synthetic peptide”
GGGKSGGGKSGGGKS

SEQ ID NO: 41 - protein, 15 aa, Artificial Sequence
  source: /note=“Description of Artificial Sequence Synthetic peptide”
GKGKSGKGKSGKGKS

SEQ ID NO: 42 - protein, 15 aa, Artificial Sequence
  source: /note=“Description of Artificial Sequence Synthetic peptide”
GGGKSGGKGSGKGGS

SEQ ID NO: 43 - protein, 15 aa, Artificial Sequence
  source: /note=“Description of Artificial Sequence Synthetic peptide”
GKPGSGKPGSGKPGS

SEQ ID NO: 44 - protein, 20 aa, Artificial Sequence
  source: /note=“Description of Artificial Sequence Synthetic peptide”
GKPGSGKPGSGKPGSGKPGS

SEQ ID NO: 45 - protein, 20 aa, Artificial Sequence
  source: /note=“Description of Artificial Sequence Synthetic peptide”
GKGKSGKGKSGKGKSGKGKS

SEQ ID NO: 46 - protein, 14 aa, Artificial Sequence
  source: /note=“Description of Artificial Sequence Synthetic peptide”
STAGDTHLGGEDFD

SEQ ID NO: 47 - protein, 15 aa, Artificial Sequence
  source: /note=“Description of Artificial Sequence Synthetic peptide”
GEGGSGEGGSGEGGS

SEQ ID NO: 48 - protein, 15 aa, Artificial Sequence
  source: /note=“Description of Artificial Sequence Synthetic peptide”
GGEGSGGEGSGGEGS

SEQ ID NO: 49 - protein, 15 aa, Artificial Sequence
  source: /note=“Description of Artificial Sequence Synthetic peptide”
GEGESGEGESGEGES

SEQ ID NO: 50 - protein, 15 aa, Artificial Sequence
  source: /note=“Description of Artificial Sequence Synthetic peptide”
GGGESGGEGSGEGGS

SEQ ID NO: 51 - protein, 20 aa, Artificial Sequence
  source: /note=“Description of Artificial Sequence Synthetic peptide”
GEGESGEGESGEGESGEGES

SEQ ID NO: 52 - protein, 18 aa, Artificial Sequence
  source: /note=“Description of Artificial Sequence Synthetic peptide”
GSTSGSGKPGSGEGSTKG

SEQ ID NO: 53 - protein, 19 aa, Artificial Sequence
  source: /note=“Description of Artificial Sequence Synthetic peptide”
PRGASKSGSASQTGSAPGS

SEQ ID NO: 54 - protein, 19 aa, Artificial Sequence
  source: /note=“Description of Artificial Sequence Synthetic peptide”
GTAAAGAGAAGGAAAGAAG

SEQ ID NO: 55 - protein, 19 aa, Artificial Sequence
  source: /note=“Description of Artificial Sequence Synthetic peptide”
GTSGSSGSGSGGSGSGGGG

SEQ ID NO: 56 - protein, 20 aa, Artificial Sequence
  source: /note=“Description of Artificial Sequence Synthetic peptide”
GKPGSGKPGSGKPGSGKPGS

SEQ ID NO: 57 - protein, 4 aa, Artificial Sequence
  source: /note=“Description of Artificial Sequence Synthetic peptide”
GSGS

SEQ ID NO: 58 - protein, 10 aa, Artificial Sequence
  source: /note=“Description of Artificial Sequence Synthetic peptide”
APAPAPAPAP

SEQ ID NO: 59 - protein, 20 aa, Artificial Sequence
  source: /note=“Description of Artificial Sequence Synthetic peptide”
APAPAPAPAPAPAPAPAPAP

SEQ ID NO: 60 - protein, 32 aa, Artificial Sequence
  source: /note=“Description of Artificial Sequence Synthetic polypeptide”
AEAAAKEAAAKEAAAAKEAAAAKEAAAAKAAA

SEQ ID NO: 61 - protein, 10 aa, Artificial Sequence
  source: /note=“Description of Artificial Sequence Synthetic peptide”
GGGGSGGGGS

SEQ ID NO: 62 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 12 aa, Artificial Sequence
  source: /note=“Description of Artificial Sequence Synthetic peptide”
VYYGSNYWYFDV

SEQ ID NO: 65 - protein, 10 aa, Artificial Sequence
  source: /note=“Description of Artificial Sequence Synthetic peptide”
RASSSVSYMH

SEQ ID NO: 66 - protein, 13 aa, Homo sapiens
SEDRYRNPKGSAC

SEQ ID NO: 67 - protein, 5 aa, Homo sapiens
EMENP

SEQ ID NO: 68 - protein, 5 aa, Homo sapiens
GFSTL

(19) United States
(12) Patent Application Publication
(10) Pub. No.: US 2025/0019455 A1
(43) Pub. Date: Jan. 16, 2025

(54) PROTEINS COMPRISING CD20 BINDING DOMAINS, AND USES THEREOF

(71) Applicant: PHARMACEUTICA NV, Beerse (BE)

(72) Inventors: Srinivasa R. BANDI, Fremont, CA (US); Jun CHEN, New Hope, PA (US); Jinquan LUO, Malvern, PA (US); Elisabeth Geyer PRINSLOW, Harleysville, PA (US); Sanjaya SINGH, Blue Bell, PA (US); Danlin YANG, Philadelphia, PA (US)

(21) Appl. No.: 18/694,091

(22) PCT Filed: Mar. 23, 2022
(86) PCT No.: PCT/EP2022/057652

§ 371 Date: Mar. 21, 2024

Related U.S. Application Data
(60) Provisional application No. 63/247,874, filed on Sep. 24, 2021.

(51) Int. Cl. C07K 16/28; A61P 35/00

(52) U.S. Cl. CPC C07K 16/2887; A61P 35/00; C07K 16/2809; C07K 2317/31; C07K 2317/526; C07K 2317/565; C07K 2317/732; C07K 2317/92

(57) ABSTRACT

The invention provides antigen binding domains that bind Cluster of Differentiation 20 protein (CD20), proteins comprising the antigen binding domains that bind CD20, polynucleotides encoding them, vectors, host cells, methods of making and using the same.

### CROSS-REFERENCE TO RELATED APPLICATIONS

This application claims the benefit of U.S. Provisional Application Ser. No. 63/247,874 filed 24 Sep. 2021. The entire content of the aforementioned application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 25, 2022, is named PRD4184WOPCT1_SL.txt and is 35,010 bytes in size.

### TECHNICAL FIELD

The invention provides antigen binding domains that bind Cluster of Differentiation 20 protein (CD20), proteins comprising the antigen binding domains that bind CD20, polynucleotides encoding them, vectors, host cells, and methods of making and using the same.

### BACKGROUND

Non-Hodgkin lymphoma (NHL) accounts for about 4% of all cancers. Despite improvements in available therapies, relapsed/refractory (r/r) NHLs are characterized by uniformly poor prognosis. Adoptive immunotherapy using T cells genetically engineered to express a chimeric antigen receptor (CAR) has shown promising results for the treatment of CD19-positive B cell malignancies. However, even with an initial overall response rate of approximately 60-80%, only 40% of patients achieve long-term, complete remission [1, 2]. There is emerging clinical data indicating disease relapse due to CD19 antigen loss in both acute lymphoblastic leukemia (ALL) and diffuse large B-cell lymphoma (DLBCL) patients [2, 4]. Accordingly, there is a need for targeting novel surface antigens.

B cells, or B lymphocytes, are central components of adaptive immunity, responding to several different pathogens by producing antibodies, performing the role of antigen-presenting cells, secreting cytokines, and developing into memory B cells after activation [5]. B cells circulate in the blood and lymphatic systems. In the lymphoid organs, a B cell encounters its cognate antigen, and together with an additional signal from a T helper cell, the B cell can differentiate into effector plasma cells. These cells secrete specific antibodies that will circulate in the blood to target and eliminate antigens or pathogens [6].

CD20 is highly expressed in a wide range of B-cell lymphomas. There is a need for novel CD20 binding domains for therapeutic and diagnostic purposes.

### SUMMARY

Table 1a and Table 1b provide a summary of CDR and VH and VL sequences of some exemplary CD20-specific antibodies described herein:

TABLE la
CDR sequences of exemplary mAbs generated against human CD20

| ID | HC-CDR1 | HC-CDR2 | HC-CDR3 | LC-CDR1 | LC-CDR2 | LC-CDR3 |
|---|---|---|---|---|---|---|
| C20B22 | GYTFTSYN | AIYPGNGDT | STYYGGDW | RASSSVSYI | ATSNLAS | QQWTSNPP |
| | MH (SEQ ID | S (SEQ ID | YFNV (SEQ | H (SEQ ID | (SEQ ID NO: | T (SEQ ID |
| | NO: 1) | NO: 2) | ID NO: 3) | NO: 4) | 5) | NO: 6) |
| C20B648 | GYTFSSYN | AIYPGAGDT | SNYYGSSG | RASLSVSSM | ATSNLAS | QQWIFNPPT |
| | MH (SEQ ID | S (SEQ ID | WYFDV | H (SEQ ID | (SEQ ID NO: | (SEQ ID NO: |
| | NO: 7) | NO: 8) | (SEQ ID | NO: 10) | 5) | 11) |
| | | | NO: 9) | | | |
| 5010GL | GYTFTSYN | AIYPGNGDT | VYYGSNYW | RASSSVSY | ATSNLAS | QQWIFNPPT |
| | MH (SEQ ID | S (SEQ ID | YFDV (SEQ | MH (SEQ ID | (SEQ ID NO: | (SEQ ID NO: |
| | NO: 1) | NO: 2) | ID NO: 64) | NO: 65) | 5) | 11 |
| 4A16GL | GYTFSSYN | AIYPGNGDT | SNYYGSSG | RASLSVSSM | ATSNLAS | QQWIFNPPT |
| | MH (SEQ ID | S (SEQ ID | WYFDV | H (SEQ ID | (SEQ ID NO: | (SEQ ID NO: |
| | NO: 7) | NO: 2) | (SEQ ID | NO: 10) | 5) | 11 |
| | | | NO: 9) | | | |

TABLE 1b
VH and VL sequences of exemplary
mAbs generated against human CD20

| | VH amino | VH DNA | VL amino | VL DNA |
|---|---|---|---|---|
| | acid sequence | sequence | acid sequence | sequence |
| ID | SEQ ID NO | SEQ ID NO | SEQ ID NO | SEQ ID NO |
| C20B22 | 12 | 13 | 14 | 15 |
| C20B648 | 16 | 17 | 18 | 19 |
| 5O10GL | 20 | 21 | 22 | 23 |
| 4A16GL | 24 | 25 | 26 | 27 |

The disclosure provides an isolated protein comprising an antigen binding domain that binds CD20, wherein the antigen binding domain that binds CD20 comprises:
a) a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14;
b) a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18;
c) a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22; or
d) a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26;

The disclosure also provides an isolated antigen binding domain that binds Cluster of Differentiation 20 protein (CD20), wherein the antigen binding domain that binds CD20 comprises a heavy chain variable region (VH) of SEQ ID NO: 12, 16, 20, or 24 and a light chain variable region (VL) of SEQ ID NO: 14, 18, 22 or 26.

In some embodiments, the isolated protein comprises the HCDR1, the HCDR2, the HCDR3, the LCDR1, the LCDR2 and the LCDR3 of
a) SEQ ID NOs: 1, 2, 3, 4, 5 and 6, respectively;
b) SEQ ID NOs: 7, 8, 9, 10, 5 and 11, respectively;
c) SEQ ID NOs: 1, 2, 64, 65, 5 and 11, respectively; or
d) SEQ ID NOs: 7, 2, 9, 10, 5 and 11, respectively.

In some embodiments, the antigen binding domain that binds CD20 is a scFv, a (scFv)₂, a Fv, a Fab, a F(ab′)₂, a Fd, a dAb or a VHH. In some embodiments, the antigen binding domain that binds CD20 is the Fab. In some embodiments, the antigen binding domain that binds CD20 is the VHH. In some embodiments, the antigen binding domain that binds CD20 is the scFv. In certain embodiments, the scFv comprises, from the N- to C-terminus, a VH, a first linker (L1) and a VL (VH-L1-VL) or the VL, the L1 and the VH (VL-L1-VH). In certain embodiments, the L1 comprises
a) about 5-50 amino acids;
b) about 5-40 amino acids;
c) about 10-30 amino acids; or
d) about 10-20 amino acids.

In certain embodiments, the L1 comprises an amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61. In some embodiments, the antigen binding domain that binds CD20 comprises the VH of SEQ ID NOs: 12, 16, 20, or 24 and the VL of SEQ ID NOs: 14, 18, 22 or 26.

In certain embodiments, the antigen binding domain that binds CD20 comprises:
a) the VH of SEQ ID NO: 12 and the VL of SEQ ID NO: 14;
b) the VH of SEQ ID NO: 16 and the VL of SEQ ID NO: 18;
c) the VH of SEQ ID NO: 20 and the VL of SEQ ID NO: 22; or
d) the VH of SEQ ID NO: 24 and the VL of SEQ ID NO: 26.

In various embodiments, the isolated protein comprises an antigen binding domain that binds Cluster of Differentiation 20 protein (CD20), wherein the antigen binding domain that binds CD20 comprises a heavy chain variable region (VH) of SEQ ID NO: 12 and a light chain variable region (VL) of SEQ ID NO: 14; a heavy chain variable region (VH) of SEQ ID NO: 16 and a light chain variable region (VL) of SEQ ID NO: 18; a heavy chain variable region (VH) of SEQ ID NO: 20 and a light chain variable region (VL) of SEQ ID NO: 22; or a heavy chain variable region (VH) of SEQ ID NO: 24 and a light chain variable region (VL) of SEQ ID NO: 26.

In various embodiments, the antigen binding domain that binds CD20 is a scFv, a (scFv)₂, a Fv, a Fab, a F(ab′)₂, a Fd, a dAb or a VHH. In various embodiments, the antigen binding domain that binds CD20 is the Fab. In various embodiments, the antigen binding domain that binds CD20 is the VHH. In various embodiments, the antigen binding domain that binds CD20 is the scFv. In various embodiments, the scFv comprises, from the N- to C-terminus, a VH, a first linker (L1) and a VL (VH-L1-VL) or the VL, the L1 and the VH (VL-L1-VH).

In some embodiments, the L1 comprises
a) about 5-50 amino acids;
b) about 5-40 amino acids;
c) about 10-30 amino acids; or
d) about 10-20 amino acids.

In some embodiments, the L1 comprises an amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61. In some embodiments, the antigen binding domain that binds CD20 comprises the VH of SEQ ID NOs: 12, 16, 20, or 24 and the VL of SEQ ID NOs: 14, 18, 22 or 26.

In various embodiments, the antigen binding domain that binds CD20 comprises:
a) the VH of SEQ ID NO: 12 and the VL of SEQ ID NO: 14;
b) the VH of SEQ ID NO: 16 and the VL of SEQ ID NO: 18;
c) the VH of SEQ ID NO: 20 and the VL of SEQ ID NO: 22; or
d) the VH of SEQ ID NO: 24 and the VL of SEQ ID NO: 26.

In some embodiments, the protein is conjugated to a half-life extending moiety.

In some embodiments, the half-life extending moiety is an immunoglobulin (Ig), a fragment of the Ig, an Ig constant region, a fragment of the Ig constant region, a Fc region, transferrin, albumin, an albumin binding domain or polyethylene glycol.

In some embodiments, the isolated protein is a monospecific protein. In some embodiments, the isolated protein is a multispecific protein. In some embodiments, the multispecific protein is a bispecific protein. In some embodiments, the multispecific protein is a trispecific protein.

In some embodiments, the isolated protein further comprises an immunoglobulin (Ig) constant region or a fragment of the Ig constant region thereof. In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises the CH2 domain and the CH3 domain. In some embodiments, the fragment of the Ig constant region comprises at least portion of a hinge, the CH2 domain and the CH3 domain. In some embodiments, the fragment of the Ig constant region comprises a hinge, the CH2 domain and the CH3 domain. In some embodiments, the antigen binding domain that binds CD20 is conjugated to the N-terminus of the Ig constant region or the fragment of the Ig constant region. In some embodiments, the antigen binding domain that binds CD20 is conjugated to the C-terminus of the Ig constant region or the fragment of the Ig constant region.

In some embodiments, the antigen binding domain that binds CD20 is conjugated to the Ig constant region or the fragment of the Ig constant region via a second linker (L2). In certain embodiments, the L2 comprises the amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61. In some embodiments, the multispecific protein comprises an antigen binding domain that binds an antigen on a lymphocyte.

In some embodiments, the lymphocyte is a T cell. In some embodiments, the T cell is a CD8⁺ T cell. In some embodiments, the lymphocyte is a natural killer (NK) cell.

In various embodiments, the multispecific protein comprises an antigen binding domain that binds CD3, CD3 epsilon (CD3ε), CD8, K12L4, NKG2E, NKG2D, NKG2F, BTNL3, CD186, BTNL8, PD-1, CD195, or NKG2C. In some embodiments, the multispecific protein comprises an antigen binding domain that binds CD3ε. In some embodiments, the Ig constant region or the fragment of the Ig constant region is an IgG1, an IgG2, an IgG3 or an IgG4 isotype. In some embodiments, the Ig constant region or the fragment of the Ig constant region is an IgG1 isotype.

In some embodiments, the Ig constant region or the fragment of the Ig constant region comprises at least one mutation that results in reduced binding of the protein to a Fcγ receptor (FcγR). In various embodiments, the at least one mutation that results in reduced binding of the protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the Ig constant region or the fragment of the Ig constant region comprises at least one mutation that results in enhanced binding of the protein to the FcγR. In some embodiments, the at least one mutation that results in enhanced binding of the protein to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In some embodiments, the FcγR is FcγRI, FcγRIIA, FcγRIIB or FcγRIII, or any combination thereof.

In some embodiments, the Ig constant region of the fragment of the Ig constant region comprises at least one mutation that modulates a half-life of the protein. In some embodiments, the at least one mutation that modulates the half-life of the protein is selected from the group consisting of H435A, P257I/N434H, D376V/N434H, M252Y/S254T/T256E/H433K/N434F, T308P/N434A and H435R, wherein residue numbering is according to the EU index. In some embodiments, the protein comprises at least one mutation in a CH3 domain of the Ig constant region.

In some embodiments, the at least one mutation in the CH3 domain of the Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the Ig constant region or the fragment of the Ig constant region comprises at least one mutation that results in enhanced binding of the protein to the FcγR. In some embodiments, the at least one mutation that results in enhanced binding of the protein to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In some embodiments, the FcγR is FcγRI, FcγRIIA, FcγRIIB or FcγRIII, or any combination thereof. In some embodiments, the Ig constant region of the fragment of the Ig constant region comprises at least one mutation that modulates a half-life of the protein.

In various embodiments, the at least one mutation that modulates the half-life of the protein is selected from the group consisting of H435A, P257I/N434H, D376V/N434H, M252Y/S254T/T256E/H433K/N434F, T308P/N434A and H435R, wherein residue numbering is according to the EU index. In various embodiments, the protein comprises at least one mutation in a CH3 domain of the Ig constant region. In some embodiments, the at least one mutation in the CH3 domain of the Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the at least one mutation in the CH3 domain of the Ig constant region is selected from the group consisting of T350V. In some embodiments, the at least one mutation in the CH3 domain of the Ig constant region is selected from the group consisting of L351Y. In some embodiments, the at least one mutation in the CH3 domain of the Ig constant region is selected from the group consisting of F405A. In some embodiments, the at least one mutation in the CH3 domain of the Ig constant region is selected from the group consisting of Y407V. In some embodiments, the at least one mutation in the CH3 domain of the Ig constant region is selected from the group consisting of T366Y. In some embodiments, the at least one mutation in the CH3 domain of the Ig constant region is selected from the group consisting of T366W.

In some embodiments, the multispecific protein comprises a first antigen binding domain that binds CD20 and a second antigen binding domain that binds a lymphocyte antigen.

In some embodiments, the lymphocyte antigen is a T cell antigen. In some embodiments, the T cell antigen is a CD8⁺ T cell antigen. In some embodiments, the lymphocyte antigen is a NK cell antigen.

In some embodiments, the lymphocyte antigen is CD3, CD3 epsilon (CD3ε), CD8, KI2L4, NKG2E, NKG2D, NKG2F, BTNL3, CD186, BTNL8, PD-1, CD195, or NKG2C. In some embodiments, the lymphocyte antigen is CD3ε. In some embodiments, the first antigen binding domain that binds CD20 and/or the second antigen binding domain that binds the lymphocyte antigen comprise a scFv, a (scFv)₂, a Fv, a Fab, a F(ab′)₂, a Fd, a dAb or a VHH. In some embodiments, the first antigen binding domain that binds CD20 and/or the second antigen binding domain that binds the lymphocyte antigen comprise the Fab. In some embodiments, the first antigen binding domain that binds CD20 and/or the second antigen binding domain that binds the lymphocyte antigen comprise the VHH. In some embodiments, the first antigen binding domain that binds CD20 and/or the second antigen binding domain that binds the lymphocyte antigen comprise the scFv.

In some embodiments, the scFv comprises, from the N- to C-terminus, a VH, a first linker (L1) and a VL (VH-L1-VL) or the VL, the L1 and the VH (VL-L1-VH). In some embodiments, the L1 comprises
a) about 5-50 amino acids;
b) about 5-40 amino acids;
c) about 10-30 amino acids; or
d) about 10-20 amino acids.

In some embodiments, the L1 comprises the amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61. In some embodiments, the first antigen binding domain that binds CD20 comprises the HCDR1 of SEQ ID NOs: 1 or 7, the HCDR2 of SEQ ID NOs: 2 or 8, the HCDR3 of SEQ ID NOs: 3, 9 or 64, the LCDR1 of SEQ ID NOs: 4, 10 or 65, the LCDR2 of SEQ ID NO: 5, and the LCDR3 of SEQ ID NOs: 6 or 11. In some embodiments, the first antigen binding domain that binds CD20 comprises the HCDR1, the HCDR2, the HCDR3, the LCDR1, the LCDR2 and the LCDR3 of
a) SEQ ID NOs: 1, 2, 3, 4, 5 and 6, respectively;
b) SEQ ID NOs: 7, 8, 9, 10, 5 and 11, respectively;
c) SEQ ID NOs: 1, 2, 64, 65, 5 and 11, respectively; or
d) SEQ ID NOs: 7, 2, 9, 10, 5 and 11, respectively.

In some embodiments, the first antigen binding domain that binds CD20 comprises
a) the VH of SEQ ID NO: 12 and the VL of SEQ ID NO: 14;
b) the VH of SEQ ID NO: 16 and the VL of SEQ ID NO: 18;
c) the VH of SEQ ID NO: 20 and the VL of SEQ ID NO: 22; or
d) the VH of SEQ ID NO: 24 and the VL of SEQ ID NO: 26.

In some embodiments, the first antigen binding domain that binds CD20 comprises the VH of SEQ ID NOs: 12, 16, 20, or 24 and the VL of SEQ ID NOs: 14, 18, 22 or 26.

In some embodiments, the first antigen binding domain that binds CD20 comprises:
a) the VH of SEQ ID NO: 12 and the VL of SEQ ID NO: 14;
b) the VH of SEQ ID NO: 16 and the VL of SEQ ID NO: 18;
c) the VH of SEQ ID NO: 20 and the VL of SEQ ID NO: 22; or
d) the VH of SEQ ID NO: 24 and the VL of SEQ ID NO: 26.

In some embodiments, the second antigen binding domain that binds the lymphocyte antigen comprises an antigen binding domain that binds CD3ε.

In some embodiments, the first antigen binding domain that binds CD20 is conjugated to a first immunoglobulin (Ig) constant region or a fragment of the first Ig constant region and/or the second antigen binding domain that binds the lymphocyte antigen is conjugated to a second immunoglobulin (Ig) constant region or a fragment of the second Ig constant region. In some embodiments, the multispecific protein further comprises a second linker (L2) between the first antigen binding domain that binds CD20 and the first Ig constant region or the fragment of the first Ig constant region and the second antigen binding domain that binds the lymphocyte antigen and the second Ig constant region or the fragment of the second Ig constant region. In certain embodiments, the L2 comprises the amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61.

In some embodiments, the first Ig constant region or the fragment of the first Ig constant region and the second Ig constant region or the fragment of the second Ig constant region is an IgG1, an IgG2, and IgG3 or an IgG4 isotype.

In some embodiments, the first Ig constant region or the fragment of the first Ig constant region and the second Ig constant region or the fragment of the second Ig constant region comprises at least one mutation that results in reduced binding of the multispecific protein to a FcγR.

In some embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR). In certain embodiments, the at least one mutation that results in enhanced binding of the multispecific protein to the FcγR is selected from the group consisting of S239D/1332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index. In some embodiments, the FcγR is FcγRI, FcγRIIA, FcγRIIB or FcγRIII, or any combination thereof. In some embodiments, the first Ig constant region or the fragment of the first Ig constant region and the second Ig constant region or the fragment of the second Ig constant region comprises at least one mutation that modulates a half-life of the multispecific protein. In some embodiments, the at least one mutation that modulates the half-life of the multispecific protein is selected from the group consisting of H435A, P257I/N434H, D376V/N434H, M252Y/S254T/T256E/H433K/N434F, T308P/N434A and H435R, wherein residue numbering is according to the EU index.

In some embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region. In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the first Ig constant region or the fragment of the first Ig constant region and the second Ig constant region or the fragment of the second Ig constant region comprise the following mutations:
a) L235A_L235A_D265S_T350V_L351Y_F405A_Y407V in the first Ig constant region and L235A_L235A_D265S_T350V_T366L_K392L_T394W in the second Ig constant region; or
b) L235A_L235A_D265S_T350V_T366L_K392L_T394W in the first Ig constant region and L235A_L235A_D265S_T350V_L351Y_F405A_Y407V in the second Ig constant region.

Also provided is an immunoconjugate comprising any of the above isolated proteins conjugated to a therapeutic agent or an imaging agent. Also provided is a pharmaceutical composition comprising any of the above isolated proteins and a pharmaceutically acceptable carrier. Also provided is a polynucleotide encoding any of the above isolated proteins, a vector encoding the polynucleotide, and a host cell comprising the vector. Also provided is a method of producing the isolated protein comprising culturing the host cell in conditions that the protein is expressed, and recovering the protein produced by the host cell.

Also provided is an immunoconjugate comprising any of the above isolated multispecific proteins conjugated to a therapeutic agent or an imaging agent. Also provided is a pharmaceutical composition comprising the isolated multispecific protein and a pharmaceutically acceptable carrier. Also provided is a polynucleotide encoding the isolated multispecific protein. Also provided is a vector comprising the polynucleotide. Also provided is a host cell comprising the vector. Also provided is a method of producing the isolated multispecific protein comprising culturing the host cell in conditions that the multispecific protein is expressed, and recovering the multispecific protein produced by the host cell.

Also provided is a method of treating a CD20 expressing cancer in a subject, comprising administering a therapeutically effective amount of the isolated protein, the isolated multispecific protein, the immunoconjugate, or the pharmaceutical composition to the subject for a time sufficient to treat the CD20 expressing cancer.

Also provided is a method of reducing the amount of CD20 expressing tumor cells in a subject, comprising administering a therapeutically effective amount of the isolated protein, the isolated multispecific protein, the immunoconjugate, or the pharmaceutical composition to the subject for a time sufficient to treat the CD20 expressing cancer.

Also provided is a method of preventing establishment of a CD20 expressing cancer in a subject, comprising administering a therapeutically effective amount of the isolated protein, the isolated multispecific protein, the immunoconjugate, or the pharmaceutical composition to the subject for a time sufficient to treat the CD20 expressing cancer.

Also provided is a method of treating a noncancerous condition in a subject at risk of developing a CD20 expressing cancer, comprising administering a therapeutically effective amount of the isolated protein, the isolated multispecific protein, the immunoconjugate, or the pharmaceutical composition to the subject for a time sufficient to treat the CD20 expressing cancer.

In some embodiments, the CD20 cancer is relapsed, refractory, or malignant cancer, or any combination thereof. In some embodiments, the isolated protein or the isolated multispecific protein is administered in combination with a second therapeutic agent. In certain embodiments, the second therapeutic agent is surgery, chemotherapy, androgen deprivation therapy or radiation, or any combination thereof.

Also provided is a kit comprising the isolated protein, the isolated multispecific protein, the immunoconjugate, or the pharmaceutical composition.

Also provided is an anti-idiotypic antibody binding to the isolated protein.

The disclosure also provides isolated antigen binding domains that bind CD20 comprising certain VH and VL amino acid sequences.

The disclosure also provides an isolated multispecific protein comprising an antigen binding domain that binds Cluster of Differentiation 20 protein (CD20), wherein the antigen binding domain that binds CD20 comprises:
a) a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14;
b) a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18;
c) a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22; or
d) a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26;
The disclosure also provides an isolated multispecific protein comprising an antigen binding domain that binds Cluster of Differentiation 20 protein (CD20), wherein the antigen binding domain that binds CD20 comprises a heavy chain variable region (VH) of SEQ ID NO: 12, 16, 20, or 24 and a light chain variable region (VL) of SEQ ID NO: 14, 18, 22 or 26.

The disclosure also provides isolated multispecific protein comprising an antigen binding domain that bind CD20 comprising certain VH and VL amino acid sequences.

The disclosure also provides an immunoconjugate comprising the isolated antigen binding domain that binds CD20 of the disclosure.

The disclosure also provides an immunoconjugate comprising the isolated protein comprising the antigen binding domain that binds CD20 of the disclosure.

The disclosure also provides an immunoconjugate comprising the isolated multispecific protein comprising the antigen binding domain that binds CD20 of the disclosure.

The disclosure also provides a pharmaceutical composition comprising the isolated antigen binding domain that binds CD20 of the disclosure.

The disclosure also provides a pharmaceutical composition comprising the isolated protein comprising the antigen binding domain that binds CD20 of the disclosure.

The disclosure also provides a pharmaceutical composition comprising the isolated multispecific protein comprising the antigen binding domain that binds CD20 of the disclosure.

The disclosure also provides an isolated polynucleotide encoding the isolated antigen binding domain that binds CD20 of the disclosure.

The disclosure also provides an isolated polynucleotide encoding the isolated protein comprising the antigen binding domain that binds CD20 of the disclosure.

The disclosure also provides an isolated polynucleotide encoding the isolated multispecific protein comprising the antigen binding domain that binds CD20 of the disclosure.

The disclosure also provides a vector comprising the polynucleotide of the disclosure.

The disclosure also provides a host cell comprising the polynucleotide or the vector of the disclosure.

The disclosure also provides a method of treating a CD20 expressing cancer in a subject, comprising administering a therapeutically effective amount of the antigen binding domain that binds CD20, the protein comprising the antigen binding domain that binds CD20, the multispecific protein comprising the antigen binding domain that binds CD20, the immunoconjugate of the disclosure or the pharmaceutical composition of the disclosure to the subject in need thereof for a time sufficient to treat the CD20 expressing cancer.

The disclosure also provides a method of reducing the amount of CD20 expressing tumor cells in a subject, comprising administering to the subject the antigen binding domain that binds CD20, the protein comprising the antigen binding domain that binds CD20, the multispecific protein comprising the antigen binding domain that binds CD20, the immunoconjugate of the disclosure or the pharmaceutical composition of the disclosure for a time sufficient to reduce the amount of CD20 expressing tumor cells.

The disclosure also provides a method of preventing establishment of a CD20 expressing cancer in a subject, comprising administering the antigen binding domain that binds CD20, the protein comprising the antigen binding domain that binds CD20, the multispecific protein comprising the antigen binding domain that binds CD20, the immunoconjugate of the disclosure or the pharmaceutical composition of the disclosure to the subject in need thereof to prevent establishment of the CD20 expressing cancer in the subject.

The disclosure also provides a method of treating a noncancerous condition in a subject at risk of developing a CD20 expressing cancerous condition, comprising administering the antigen binding domain that binds CD20, the protein comprising the antigen binding domain that binds CD20, the multispecific protein comprising the antigen binding domain that binds CD20, the immunoconjugate of the disclosure or the pharmaceutical composition of the disclosure to the subject in need thereof to treat the noncancerous condition.

The disclosure also provides a method of treating B-cell lymphoma in a subject, comprising administering a therapeutically effective amount of the antigen binding domain that binds CD20, the protein comprising the antigen binding domain that binds CD20, the multispecific protein comprising the antigen binding domain that binds CD20, the immunoconjugate of the disclosure or the pharmaceutical composition of the disclosure to the subject in need thereof for a time sufficient to treat the B-cell lymphoma.

The disclosure also provides a kit comprising the antigen binding domain that binds CD20, the protein comprising the antigen binding domain that binds CD20, the multispecific protein comprising the antigen binding domain that binds CD20, the immunoconjugate of the disclosure or the pharmaceutical composition of the disclosure.

The disclosure also provides an anti-idiotypic antibody binding to the antigen binding domain that binds CD20 of the disclosure.

### BRIEF DESCRIPTION OF THE DRAWINGS

FIG. 1. Depiction of a exemplary CD79b×CD20×CD3 trispecific antibody. FIG. 1 discloses SEQ ID NO: 34.

FIGS. 2A-2D. Binding affinities of selected CD79b×CD3 bsAbs in the HLB-1 cell line (FIG. 2A); the OCI-LY10 cell line (FIG. 2B); the Carnaval cell line (FIG. 2C); and the WILL-2 cell line (FIG. 2D). Circles correspond to the 79C3B646 bsAb; triangles correspond to the 79C3B651 bsAb; and diamonds correspond to the 79C3B601 bsAb.

FIGS. 3A-3D. Binding affinities of selected CD79b×CD20×CD3 trispecific antibodies in the HLB-1 cell line (FIG. 3A); the OCI-LY10 cell line (FIG. 3B); the Carnaval cell line (FIG. 3C); and the WILL-2 cell line (FIG. 3D). Solid circles correspond to the 79C3B646 bsAb control; solid triangles correspond to the 79C3B651 bsAb control; and solid diamonds correspond to the 79C3B601 bsAb control. Open triangles correspond to trispecific antibody C923B38; open diamonds correspond to trispecific antibody C923B74; asterisks correspond to trispecific antibody C923B9; and X corresponds to control null trispecific antibody C923B98.

FIGS. 4A-4I. Binding kinetics of selected CD79b×CD3 bsAbs on DLBCL cell lines. Binding kinetics of the three selected bsAbs in HBL-1 cells at 300 nm (FIG. 4A). Binding kinetics of the three selected bsAbs in HBL-1 cells at 60 nm (FIG. 4B). Binding kinetics of the three selected bsAbs in HBL-1 cells at 12 nm (FIG. 4C). Binding kinetics of the three selected bsAbs in Carnaval cells at 300 nm (FIG. 4D). Binding kinetics of the three selected bsAbs in Carnaval cells at 60 nm (FIG. 4E). Binding kinetics of the three selected bsAbs in Carnaval cells at 12 nm (FIG. 4F). Binding kinetics of the three selected bsAbs in OCI-LY10 cells at 300 nm (FIG. 4G). Binding kinetics of the three selected bsAbs in OCI-LY10 cells at 60 nm (FIG. 4H). Binding kinetics of the three selected bsAbs in OCI-LY10 cells at 12 nm (FIG. 4I). Inverted triangles correspond to the 79C3B646 bsAb; diamonds correspond to the 79C3B651 bsAb; and squares correspond to the 79C3B601 bsAb.

FIGS. 5A-5I. Binding kinetics of selected CD79b×CD20×CD3 trispecific antibodies on DLBCL cell lines. Binding kinetics of the selected antibodies in HBL-1 cells at 300 nm (FIG. 5A). Binding kinetics of the selected antibodies in HBL-1 cells at 60 nm (FIG. 5B). Binding kinetics of the selected antibodies in HBL-1 cells at 12 nm (FIG. 5C). Binding kinetics of the selected antibodies in Carnaval cells at 300 nm (FIG. 5D). Binding kinetics of the selected antibodies in Carnaval cells at 60 nm (FIG. 5E). Binding kinetics of the selected antibodies in Carnaval cells at 12 nm (FIG. 5F). Binding kinetics of the selected antibodies in OCI-LY10 cells at 300 nm (FIG. 5G). Binding kinetics of the selected antibodies in OCI-LY10 cells at 60 nm (FIG. 5H). Binding kinetics of the selected antibodies in OCI-LY10 cells at 12 nm (FIG. 5I). Inverted triangles correspond to the 79C3B646 bsAb control; diamonds correspond to the 79C3B651 bsAb control; and squares correspond to the 79C3B601 bsAb control. Triangles correspond to trispecific antibody C923B38; circles correspond to trispecific antibody C923B74; squares correspond to trispecific antibody C923B99; and asterisks correspond to control null trispecific antibody C923B98.

FIGS. 6A-6D. Primary pan T-cell binding of CD79b×CD20×CD3 trispecific antibodies and CD79b×CD3 bispecific antibodies. Binding kinetics of the selected antibodies in pan T-cell donor line D221837 (FIG. 6A). Binding kinetics of the selected antibodies in pan T-cell donor line D329312 (FIG. 6B). Binding kinetics of the selected antibodies in pan T-cell donor line D329335 (FIG. 6C). Binding kinetics of the selected antibodies in pan T-cell donor line D160115 (FIG. 6D). Circles correspond to the 79C3B651 bsAb; squares correspond to the 79C3B646 bsAb; triangles correspond to the trispecific antibody C923B38; inverted triangles correspond to the trispecific antibody C923B99; diamonds correspond to the trispecific antibody C923B74.

FIGS. 7A-7B. T cell cytotoxicity of CD79b×CD20×CD3 trispecific antibodies and CD79b×CD3 bispecific antibodies. Cytotoxicity of the selected antibodies in the HEL T-cell line (FIG. 7A). Cytotoxicity of the selected antibodies in the K562 T-cell line (FIG. 7B). Shaded circles correspond to the trispecific antibody C923B74; clear circles correspond to the trispecific antibody C923B99; triangles correspond to the trispecific antibody C923B38; inverted triangles correspond to the 79C3B646 bsAb; diamonds correspond to 79C3B651 bsAb; black squares correspond to the 79C3B601 bsAb; and white squares correspond to C923B98 bsAb.

FIGS. 8A-8C. CD79b×CD20×CD3 trispecific construct mediated B cell cytotoxicity and T cell activation. Cytotoxicity in B cells (FIG. 8A); CD4⁺ T-cells (FIG. 8B) and CD8⁺ T-cells are shown for the lead antibodies.

### DETAILED DESCRIPTION

The disclosed methods may be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended, and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of”.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Activation,” “stimulation,” “activated,” or “stimulated” refer to induction of a change in the biologic state of a cell resulting in expression of activation markers, cytokine production, proliferation or mediating cytotoxicity of target cells. Cells may be activated by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or natural killer (NK) cell proliferation and/or upregulation or downregulation of key molecules.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antibody-dependent cellular cytotoxicity”, “antibody-dependent cell-mediated cytotoxicity” or “ADCC” refers to the mechanism of inducing cell death that depends upon the interaction of antibody-coated target cells with effector cells possessing lytic activity, such as NK cells, monocytes, macrophages and neutrophils via Fc gamma receptors (FcγR) expressed on effector cells.

“Antibody-dependent cellular phagocytosis” or “ADCP” refers to the mechanism of elimination of antibody-coated target cells by internalization by phagocytic cells, such as macrophages or dendritic cells.

“Antigen” refers to any molecule (e.g., protein, peptide, polysaccharide, glycoprotein, glycolipid, nucleic acid, portions thereof, or combinations thereof) capable of being bound by an antigen binding domain or a T-cell receptor that is capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, and killed or inactivated whole cells or lysates.

“Antigen binding fragment” or “antigen binding domain” refers to a portion of the protein that binds an antigen. Antigen binding fragments may be synthetic, enzymatically obtainable or genetically engineered polypeptides and include portions of an immunoglobulin that bind an antigen, such as VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each HC is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes: IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (x) and lambda (Q), based on the amino acid sequences of their constant domains.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Complement-dependent cytotoxicity” or “CDC”, refers to the mechanism of inducing cell death in which the Fc effector domain of a target-bound protein binds and activates complement component C1q which in turn activates the complement cascade leading to target cell death. Activation of complement may also result in deposition of complement components on the target cell surface that facilitate CDC by binding complement receptors (e.g., CR3) on leukocytes

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g. Lefranc et al. (2003) Dev Comp Immunol 27: 55-77; Honegger and Pluckthun, J Mol Biol (2001) 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Decrease,” “lower,” “lessen,” “reduce,” or “abate” refers generally to the ability of a test molecule to mediate a reduced response (i.e., downstream effect) when compared to the response mediated by a control or a vehicle. Exemplary responses are T cell expansion, T cell activation or T-cell mediated tumor cell killing or binding of a protein to its antigen or receptor, and enhanced binding to a Fcγ or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Differentiation” refers to a method of decreasing the potency or proliferation of a cell or moving the cell to a more developmentally restricted state.

“Encode” or “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

“Enhance,” “promote,” “increase,” “expand” or “improve” refers generally to the ability of a test molecule to mediate a greater response (i.e., downstream effect) when compared to the response mediated by a control or a vehicle. Exemplary responses are T cell expansion, T cell activation or T-cell mediated tumor cell killing or binding of a protein to its antigen or receptor, and enhanced binding to a Fcγ or enhanced Fc effector functions such as enhanced ADCC, CDC and/or ADCP. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7. 1.8, etc.).

“Expansion” refers to the outcome of cell division and cell death.

“Express” and “expression” refers the to the well-known transcription and translation occurring in cells or in vitro. The expression product, e.g., the protein, is thus expressed by the cell or in vitro and may be an intracellular, extracellular or a transmembrane protein.

“Expression vector” refers to a vector that can be utilized in a biological system or in a reconstituted biological system to direct the translation of a polypeptide encoded by a polynucleotide sequence present in the expression vector. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

“dAb” or “dAb fragment” refers to an antibody fragment composed of a VH domain (Ward et al., Nature 341:544 546 (1989)).

“Fab” or “Fab fragment” refers to an antibody fragment composed of VH, CH1, VL and CL domains.

“F(ab′)₂” or “F(ab′)₂ fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody.

“Full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Genetic modification” refers to the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to the polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

“Heterologous” refers to two or more polynucleotides or two or more polypeptides that are not found in the same relationship to each other in nature.

“Heterologous polynucleotide” refers to a non-naturally occurring polynucleotide that encodes two or more neoantigens as described herein.

“Heterologous polypeptide” refers to a non-naturally occurring polypeptide comprising two or more neoantigen polypeptides as described herein.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“In combination with” means that two or more therapeutic agents are be administered to a subject together in a mixture, concurrently as single agents or sequentially as single agents in any order.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Cluster of Differentiation 20” or “CD20” refers to an antigenic determinant known to be detectable on B cells. Human CD20 is also called membrane-spanning 4-domains, subfamily A, member 1 (MS4A1). The human and murine amino acid and nucleic acid sequences can be found in a public database, such as GenBank, UniProt and Swiss-Prot. For example, the amino acid sequence of human CD20 can be found at Accession Nos. NP_690605.1 and NP_068769.2, and the nucleic acid sequence encoding transcript variants 1 and 3 of the human CD20 can be found at Accession No. NM_152866.2 and NM_021950.3, respectively.

“Modulate” refers to either enhanced or decreased ability of a test molecule to mediate an enhanced or a reduced response (i.e., downstream effect) when compared to the response mediated by a control or a vehicle.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

“Natural killer cell” and “NK cell” are used interchangeably and synonymously herein. NK cell refers to a differentiated lymphocyte with a CD16⁺CD56⁺ and/or CD57⁺ TCR⁻ phenotype. NK cells are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

“Operatively linked” and similar phrases, when used in reference to nucleic acids or amino acids, refers to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA) and in some instances to the production of a polypeptide (i.e., expression of the open reading frame). “Operatively linked peptide” refers to a peptide in which the functional domains of the peptide are placed with appropriate distance from each other to impart the intended function of each domain.

“Pharmaceutical combination” refers to a combination of two or more active ingredients administered either together or separately.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Polynucleotide” or “nucleic acid” refers to a synthetic molecule comprising a chain of nucleotides covalently linked by a sugar-phosphate backbone or other equivalent covalent chemistry. cDNA is a typical example of a polynucleotide. Polynucleotide may be a DNA or a RNA molecule.

“Prevent,” “preventing,” “prevention,” or “prophylaxis” of a disease or disorder means preventing a disorder from occurring in a subject.

“Proliferation” refers to an increase in cell division, either symmetric or asymmetric division of cells.

“Promoter” refers to the minimal sequences required to initiate transcription. Promoter may also include enhancers or repressor elements which enhance or suppress transcription, respectively.

“Protein” or “polypeptide” are used interchangeably herein are refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Recombinant” refers to polynucleotides, polypeptides, vectors, viruses and other macromolecules that are prepared, expressed, created or isolated by recombinant means.

“Regulatory element” refers to any cis- or trans acting genetic element that controls some aspect of the expression of nucleic acid sequences.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL.

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with a K_{D} that is at least one hundred fold less than its K_{D} for binding to a non-specific antigen (e.g., BSA, casein). In the context of the CD20 antigens described here, “specific binding” refers to binding of the proteinaceous molecule to the CD20 antigen without detectable binding to a wild-type protein the antigen is a variant of.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell” and “T lymphocyte” are interchangeable and used synonymously herein. T cell includes thymocytes, naive T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4+CD8⁺ T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8⁺ cytotoxic T cell response. Also included are “regulatory T cells” or “Tregs” which refer to T cells that suppress an abnormal or excessive immune response and play a role in immune tolerance. Tregs are typically transcription factor Foxp3-positive CD4⁺ T cells and can also include transcription factor Foxp3-negative regulatory T cells that are IL-10-producing CD4⁺ T cells.

“Therapeutically effective amount” or “effective amount” as used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics include, for example, improved wellbeing of the patient, reduction of a tumor burden, arrested or slowed growth of a tumor, and/or absence of metastasis of cancer cells to other locations in the body.

“Transduction” refers to the introduction of a foreign nucleic acid into a cell using a viral vector.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid or uptake of exogenous nucleic acid, it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Mutations in the Ig constant regions are referred to as follows: L351Y_F405A_Y407V refers to L351Y, F405A and Y407V mutations in one immunoglobulin constant region. L351Y_F405A_Y407V/T394W refers to L351Y, F405A and Y407V mutations in the first Ig constant region and T394W mutation in the second Ig constant region, which are present in one multimeric protein.

### Compositions of Matter

Antigen Binding Domains that Bind CD20

The disclosure provides antigen binding domains that bind CD20, monospecific and multispecific proteins comprising the antigen binding domains that bind CD20, chimeric antigen receptors (CAR) comprising the antigen binding domains that bind CD20, polynucleotides encoding the foregoing, vectors, host cells and methods of making and using the foregoing.

The disclosure provides an isolated protein comprising an antigen binding domain that binds Cluster of Differentiation CD20 protein (CD20), wherein the antigen binding domain that binds CD20 comprises a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14;
a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18;
a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22; or
a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26.

The disclosure provides an isolated protein comprising an antigen binding domain that binds CD20, wherein the antigen binding domain that binds CD20 comprises the HCDR1, the HCDR2, the HCDR3, the LCDR1, the LCDR2 and the LCDR3 of
SEQ ID NOs: 1, 2, 3, 4, 5 and 6, respectively;
SEQ ID NOs: 7, 8, 9, 10, 5 and 11, respectively;
SEQ ID NOs: 1, 2, 64, 65, 5 and 11, respectively; or
SEQ ID NOs: 7, 2, 9, 10, 5 and 11, respectively.

The disclosure provides an isolated protein comprising an antigen binding domain that binds CD20, wherein the antigen binding domain that binds CD20 comprises the VH of SEQ ID NOs: 12, 16, 20, or 24 and the VL of SEQ ID NO: 14, 18, 22 or 26.

The disclosure provides an isolated protein comprising an antigen binding domain that binds CD20, wherein the antigen binding domain that binds CD20 comprises
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

The disclosure also provides an isolated protein comprising an antigen binding domain that binds Cluster of Differentiation 20 protein (CD20), wherein the antigen binding domain that binds CD20 comprises a heavy chain variable region (VH) of SEQ ID NO: 12 and a light chain variable region (VL) of SEQ ID NO: 14; a heavy chain variable region (VH) of SEQ ID NO: 16 and a light chain variable region (VL) of SEQ ID NO: 18; a heavy chain variable region (VH) of SEQ ID NO: 20 and a light chain variable region (VL) of SEQ ID NO: 22; or a heavy chain variable region (VH) of SEQ ID NO: 24 and a light chain variable region (VL) of SEQ ID NO: 26.

The disclosure provides an isolated protein comprising an antigen binding domain that binds CD20, wherein the antigen binding domain that binds CD20 comprises the VH of SEQ ID NOs: 12, 16, 20, or 24 and the VL of SEQ ID NO: 14, 18, 22 or 26.

The disclosure also provides an isolated protein comprising an antigen binding domain that binds CD20, wherein the antigen binding domain that binds CD20 comprises
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

In some embodiments, the antigen binding domain that binds CD20 is a scFv.

In some embodiments, the antigen binding domain that binds CD20 is a (scFv)₂.

In some embodiments, the antigen binding domain that binds CD20 is a Fv.

In some embodiments, the antigen binding domain that binds CD20 is a Fab.

In some embodiments, the antigen binding domain that binds CD20 is a F(ab′)₂.

In some embodiments, the antigen binding domain that binds CD20 is a Fd.

In some embodiments, the CD20 antigen binding domain is a dAb.

In some embodiments, the CD20 antigen binding domain is a VHH

CD20 Binding scFvs

Any of the VH and the VL domains identified herein that bind CD20 may be engineered into scFv format in either VH-linker-VL or VL-linker-VH orientation. Any of the VH and the VL domains identified herein may also be used to generate sc(Fv)₂ structures, such as VH-linker-VL-linker-VL-linker-VH, VH-linker-VL-linker-VH-linker-VL. VH-linker-VH-linker-VL-linker-VL. VL-linker-VH-linker-VH-linker-VL. VL-linker-VH-linker-VL-linker-VH or VL-linker-VL-linker-VH-linker-VH.

VH and the VL domains identified herein may be incorporated into a scFv format and the binding and thermostability of the resulting scFv to CD20 may be assessed using known methods. Binding may be assessed using ProteOn XPR36, Biacore 3000 or KinExA instrumentation, ELISA or competitive binding assays known to those skilled in the art. Binding may be evaluated using purified scFvs or E. coli supernatants or lysed cells containing the expressed scFv. The measured affinity of a test scFv to CD20 may vary if measured under different conditions (e.g., osmolarity, pH). Thus, measurements of affinity and other binding parameters (e.g., K_{D}, Kₒₙ, K_{off}) are typically made with standardized conditions and standardized buffers. Thermostability may be evaluated by heating the test scFv at elevated temperatures, such as at 50° C., 55° C. or 60° C. for a period of time, such as 5 minutes (min), 10 min, 15 min, 20 min, 25 min or 30 min and measuring binding of the test scFv to CD20. The scFvs retaining comparable binding to CD20 when compared to a non-heated scFv sample are referred to as being thermostable.

In recombinant expression systems, the linker is a peptide linker and may include any naturally occurring amino acid. Exemplary amino acids that may be included into the linker are Gly, Ser, Pro, Thr, Glu, Lys, Arg, Ile, Leu, His and The. The linker should have a length that is adequate to link the VH and the VL in such a way that they form the correct conformation relative to one another so that they retain the desired activity, such as binding to CD20.

The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that may be used are Gly rich linkers, Gly and Ser containing linkers, Gly and Ala containing linkers, Ala and Ser containing linkers, and other flexible linkers.

Other linker sequences may include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers. Exemplary linkers that may be used are shown in Table 2. Additional linkers are described for example in Int. Pat. Publ. No. WO2019/060695.

In some embodiments, the scFv comprises, from the N- to C-terminus, a VH, a first linker (L1) and a VL (VH-L1-VL).

In some embodiments, the scFv comprises, from the N- to C-terminus, the VL, the L1 and the VH (VL-L1-VH).

In some embodiments, the L1 comprises the amino acid sequence of SEQ ID NO: 28.

In some embodiments, the L1 comprises the amino acid sequence of SEQ ID NO: 29.

In some embodiments, the L1 comprises the amino acid sequence of SEQ ID NO: 30.

In some embodiments, the L1 comprises the amino acid sequence of SEQ ID NO: 31.

In some embodiments, the L1 comprises the amino acid sequence of SEQ ID NO: 32.

In some embodiments, the L1 comprises the amino acid sequence of SEQ ID NO: 33.

In some embodiments, the L1 comprises the amino acid sequence of SEQ ID NO: 34.

In some embodiments, the L1 comprises the amino acid sequence of SEQ ID NO: 35.

In some embodiments, the L1 comprises the amino acid sequence of SEQ ID NO: 36.

In some embodiments, the L1 comprises the amino acid sequence of SEQ ID NO: 37.

In some embodiments, the L1 comprises the amino acid sequence of SEQ ID NO: 38.

In some embodiments, the L1 comprises the amino acid sequence of SEQ ID NO: 39.

In some embodiments, the L1 comprises the amino acid sequence of SEQ ID NO: 40.

In some embodiments, the L1 comprises the amino acid sequence of SEQ ID NO: 41.

In some embodiments, the L1 comprises the amino acid sequence of SEQ ID NO: 42.

In some embodiments, the L1 comprises the amino acid sequence of SEQ ID NO: 43.

In some embodiments, the L1 comprises the amino acid sequence of SEQ ID NO: 44.

In some embodiments, the L1 comprises the amino acid sequence of SEQ ID NO: 45.

In some embodiments, the L1 comprises the amino acid sequence of SEQ ID NO: 46.

In some embodiments, the L1 comprises the amino acid sequence of SEQ ID NO: 47.

In some embodiments, the L1 comprises the amino acid sequence of SEQ ID NO: 48.

In some embodiments, the L1 comprises the amino acid sequence of SEQ ID NO: 49.

In some embodiments, the L1 comprises the amino acid sequence of SEQ ID NO: 50.

In some embodiments, the L1 comprises the amino acid sequence of SEQ ID NO: 51.

In some embodiments, the L1 comprises the amino acid sequence of SEQ ID NO: 52.

In some embodiments, the L1 comprises the amino acid sequence of SEQ ID NO: 53.

In some embodiments, the L1 comprises the amino acid sequence of SEQ ID NO: 54.

In some embodiments, the L1 comprises the amino acid sequence of SEQ ID NO: 55.

In some embodiments, the L1 comprises the amino acid sequence of SEQ ID NO: 56.

In some embodiments, the L1 comprises the amino acid sequence of SEQ ID NO: 57.

In some embodiments, the L1 comprises the amino acid sequence of SEQ ID NO: 58.

In some embodiments, the L1 comprises the amino acid sequence of SEQ ID NO: 59.

In some embodiments, the L1 comprises the amino acid sequence of SEQ ID NO: 60.

In some embodiments, the L1 comprises the amino acid sequence of SEQ ID NO: 61.

TABLE 2

| | | SEQ |
|---|---|---|
| | | ID |
| Linker name | Amino acid sequence | NO: |
| Linker 1 | GGSEGKSSGSGSESKSTGGS | 28 |
| Linker 2 | GGGSGGGS | 29 |
| Linker 3 | GGGSGGGSGGGS | 30 |
| Linker 4 | GGGSGGGSGGGSGGGS | 31 |
| Linker 5 | GGGSGGGSGGGSGGGSGGGS | 32 |
| Linker 6 | GGGGSGGGGSGGGGS | 33 |
| Linker 7 | GGGGSGGGGSGGGGSGGGGS | 34 |
| Linker 8 | GGGGSGGGGSGGGGSGGGGSGGGGS | 35 |
| Linker 9 | GSTSGSGKPGSGEGSTKG | 36 |
| Linker 10 | IRPRAIGGSKPRVA | 37 |
| Linker 11 | GKGGSGKGGSGKGGS | 38 |
| Linker 12 | GGKGSGGKGSGGKGS | 39 |
| Linker 13 | GGGKSGGGKSGGGKS | 40 |
| Linker 14 | GKGKSGKGKSGKGKS | 41 |
| Linker 15 | GGGKSGGKGSGKGGS | 42 |
| Linker 16 | GKPGSGKPGSGKPGS | 43 |
| Linker 17 | GKPGSGKPGSGKPGSGKPGS | 44 |
| Linker 18 | GKGKSGKGKSGKGKSGKGKS | 45 |
| Linker 19 | STAGDTHLGGEDFD | 46 |
| Linker 20 | GEGGSGEGGSGEGGS | 47 |
| Linker 21 | GGEGSGGEGSGGEGS | 48 |
| Linker 22 | GEGESGEGESGEGES | 49 |
| Linker 23 | GGGESGGEGSGEGGS | 50 |
| Linker 24 | GEGESGEGESGEGESGEGES | 51 |
| Linker 25 | GSTSGSGKPGSGEGSTKG | 52 |
| Linker 26 | PRGASKSGSASQTGSAPGS | 53 |
| Linker 27 | GTAAAGAGAAGGAAAGAAG | 54 |
| Linker 28 | GTSGSSGSGSGGSGSGGGG | 55 |
| Linker 29 | GKPGSGKPGSGKPGSGKPGS | 56 |
| Linker 30 | GSGS | 57 |
| Linker 31 | APAPAPAPAP | 58 |
| Linker 32 | APAPAPAPAPAPAPAPAPAP | 59 |
| Linker 33 | AEAAAKEAAAKEAAAAKEAAAAKEAAAAKAAA | 60 |
| Linker 34 | GGGGSGGGGS | 61 |

In some embodiments, the scFv comprises a heavy chain complementarity determining region (HCDR) 1, a HCDR2 and a HCDR3 of a heavy chain variable (VH) of SEQ ID NO: 12 and a light chain complementarity determining region (LCDR) 1, a LCDR2 and a LCDR3 of a light chain variable region (VL) of SEQ ID NO: 14.

In some embodiments, the scFv comprises a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18.

In some embodiments, the scFv comprises a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22.

In some embodiments, the scFv comprises a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26.

In some embodiments, the scFv comprises the HCDR1, the HCDR2, the HCDR3, the LCDR1, the LCDR2 and the LCDR3 of
SEQ ID NOs: 1, 2, 3, 4, 5 and 6, respectively;
SEQ ID NOs: 7, 8, 9, 10, 5 and 11, respectively;
SEQ ID NOs: 1, 2, 64, 65, 5 and 11, respectively; or
SEQ ID NOs: 7, 2, 9, 10, 5 and 11, respectively.

In some embodiments, the scFv comprises the HCDR1, the HCDR2, the HCDR3, the LCDR1, the LCDR2 and the LCDR3 of SEQ ID NOs: 1, 2, 3, 4, 5 and 6, respectively.

In some embodiments, the scFv comprises the HCDR1, the HCDR2, the HCDR3, the LCDR1, the LCDR2 and the LCDR3 of SEQ ID NOs: 7, 8, 9, 10, 5 and 11, respectively.

In some embodiments, the scFv comprises the HCDR1, the HCDR2, the HCDR3, the LCDR1, the LCDR2 and the LCDR3 of SEQ ID NOs: 1, 2, 64, 65, 5 and 11, respectively.

In some embodiments, the scFv comprises the HCDR1, the HCDR2, the HCDR3, the LCDR1, the LCDR2 and the LCDR3 of SEQ ID NOs: 7, 2, 9, 10, 5 and 11, respectively.

In some embodiments, the scFv comprises VH of SEQ ID NO: 12 and the VL of SEQ ID NO: 14.

In some embodiments, the scFv comprises VH of SEQ ID NO: 16 and the VL of SEQ ID NO: 18.

In some embodiments, the scFv comprises VH of SEQ ID NO: 20 and the VL of SEQ ID NO: 22.

In some embodiments, the scFv comprises VH of SEQ ID NO: 24 and the VL of SEQ ID NO: 26.

In some embodiments, the scFv comprises the VH of SEQ ID NOs: 12, 16, 20, or 24 and the VL of SEQ ID NOs: 14, 18, 22 or 26.

In some embodiments the scFv comprises, from the N- to C-terminus, a VH, a first linker (L1) and a VL (VH-L1-VL) or the VL, the L1 and the VH (VL-L1-VH). In certain embodiments, the L1 comprises about 5-50 amino acids; about 5-40 amino acids; about 10-30 amino acids; or about 10-20 amino acids. In certain embodiments, the L1 comprises an amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61.

Other Antigen Binding Domains that Bind CD20

Any of the VH and the VL domains identified herein that bind CD20 may also be engineered into Fab, F(ab′)₂, Fd or Fv format and their binding to CD20 and thermostability may be assessed using the assays described herein.

In some embodiments, the Fab comprises a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14.

In some embodiments, the Fab comprises a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18.

In some embodiments, the Fab comprises a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22.

In some embodiments, the Fab comprises a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26.

In some embodiments, the Fab comprises the HCDR1, the HCDR2, the HCDR3, the LCDR1, the LCDR2 and the LCDR3 of
SEQ ID NOs: 1, 2, 3, 4, 5 and 6, respectively;
SEQ ID NOs: 7, 8, 9, 10, 5 and 11, respectively;
SEQ ID NOs: 1, 2, 64, 65, 5 and 11, respectively; or
SEQ ID NOs: 7, 2, 9, 10, 5 and 11, respectively.

In some embodiments, the Fab comprises the HCDR1, the HCDR2, the HCDR3, the LCDR1, the LCDR2 and the LCDR3 of SEQ ID NOs: 1, 2, 3, 4, 5 and 6, respectively.

In some embodiments, the Fab comprises the HCDR1, the HCDR2, the HCDR3, the LCDR1, the LCDR2 and the LCDR3 of SEQ ID NOs: 7, 8, 9, 10, 5 and 11, respectively.

In some embodiments, the Fab comprises the HCDR1, the HCDR2, the HCDR3, the LCDR1, the LCDR2 and the LCDR3 of SEQ ID NOs: 1, 2, 64, 65, 5 and 11, respectively.

In some embodiments, the Fab comprises the HCDR1, the HCDR2, the HCDR3, the LCDR1, the LCDR2 and the LCDR3 of SEQ ID NOs: 7, 2, 9, 10, 5 and 11, respectively.

In some embodiments, the Fab comprises the VH of SEQ ID NO: 12 and the VL of SEQ ID NO: 14.

In some embodiments, the Fab comprises the VH of SEQ ID NO: 16 and the VL of SEQ ID NO: 18.

In some embodiments, the Fab comprises the VH of SEQ ID NO: 20 and the VL of SEQ ID NO: 22.

In some embodiments, the Fab comprises the VH of SEQ ID NO: 24 and the VL of SEQ ID NO: 26.

In some embodiments, the F(ab′)₂ comprises a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14.

In some embodiments, the F(ab′)₂ comprises a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18.

In some embodiments, the F(ab′)₂ comprises a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22.

In some embodiments, the F(ab′)₂ comprises a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26.

In some embodiments, the F(ab′)₂ comprises the HCDR1, the HCDR2, the HCDR3, the LCDR1, the LCDR2 and the LCDR3 of
SEQ ID NOs: 1, 2, 3, 4, 5 and 6, respectively;
SEQ ID NOs: 7, 8, 9, 10, 5 and 11, respectively;
SEQ ID NOs: 1, 2, 64, 65, 5 and 11, respectively; or
SEQ ID NOs: 7, 2, 9, 10, 5 and 11, respectively.

In some embodiments, the F(ab′)₂ comprises the HCDR1, the HCDR2, the HCDR3, the LCDR1, the LCDR2 and the LCDR3 of SEQ ID NOs: 1, 2, 3, 4, 5 and 6, respectively.

In some embodiments, the F(ab′)₂ comprises the HCDR1, the HCDR2, the HCDR3, the LCDR1, the LCDR2 and the LCDR3 of SEQ ID NOs: SEQ ID NOs: 7, 8, 9, 10, 5 and 11, respectively.

In some embodiments, the F(ab′)₂ comprises the HCDR1, the HCDR2, the HCDR3, the LCDR1, the LCDR2 and the LCDR3 of SEQ ID NOs: 1, 2, 64, 65, 5 and 11, respectively.

In some embodiments, the F(ab′)₂ comprises the HCDR1, the HCDR2, the HCDR3, the LCDR1, the LCDR2 and the LCDR3 of SEQ ID NOs: SEQ ID NOs: 7, 2, 9, 10, 5 and 11, respectively.

In some embodiments, the F(ab′)₂ comprises the VH of SEQ ID NO: 12 and the VL of SEQ ID NO: 14.

In some embodiments, the F(ab′)₂ comprises the VH of SEQ ID NO: 16 and the VL of SEQ ID NO: 18.

In some embodiments, the F(ab′)₂ comprises the VH of SEQ ID NO: 20 and the VL of SEQ ID NO: 22.

In some embodiments, the F(ab′)₂ comprises the VH of SEQ ID NO: 24 and the VL of SEQ ID NO: 26.

In some embodiments, the Fv comprises a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14.

In some embodiments, the Fv comprises a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18.

In some embodiments, a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22.

In some embodiments, the Fv comprises) a heavy chain complementarity determining region (HCDR) 1, a HCDR2 and a HCDR3 of a heavy chain variable (VH) of SEQ ID NO: 24 and a light chain complementarity determining region (LCDR) 1, a LCDR2 and a LCDR3 of a light chain variable region (VL) of SEQ ID NO: 26.

In some embodiments, the Fv comprises the HCDR1, the HCDR2, the HCDR3, the LCDR1, the LCDR2 and the LCDR3 of
SEQ ID NOs: 1, 2, 3, 4, 5 and 6, respectively;
SEQ ID NOs: 7, 8, 9, 10, 5 and 11, respectively;
SEQ ID NOs: 1, 2, 64, 65, 5 and 11, respectively; or
SEQ ID NOs: 7, 2, 9, 10, 5 and 11, respectively.

In some embodiments, the Fv comprises the HCDR1, the HCDR2, the HCDR3, the LCDR1, the LCDR2 and the LCDR3 of SEQ ID NOs: 1, 2, 3, 4, 5 and 6, respectively.

In some embodiments, the Fv comprises the HCDR1, the HCDR2, the HCDR3, the LCDR1, the LCDR2 and the LCDR3 of SEQ ID NOs: 7, 8, 9, 10, 5 and 11, respectively.

In some embodiments, the Fv comprises the HCDR1, the HCDR2, the HCDR3, the LCDR1, the LCDR2 and the LCDR3 of SEQ ID NOs: 1, 2, 64, 65, 5 and 11, respectively.

In some embodiments, the Fv comprises the HCDR1, the HCDR2, the HCDR3, the LCDR1, the LCDR2 and the LCDR3 of SEQ ID NOs: 7, 2, 9, 10, 5 and 11, respectively.

In some embodiments, the Fv comprises the VH of SEQ ID NO: 12 and the VL of SEQ ID NO: 14.

In some embodiments, the Fv comprises the VH of SEQ ID NO: 16 and the VL of SEQ ID NO: 18.

In some embodiments, the Fv comprises the VH of SEQ ID NO: 20 and the VL of SEQ ID NO: 22.

In some embodiments, the Fv comprises the VH of SEQ ID NO: 24 and the VL of SEQ ID NO: 26.

In some embodiments, the Fv comprises the VH of SEQ ID NOs: 12, 16, 20, or 24 and the VL of SEQ ID NOs: 14, 18, 22 or 26.

Homologous Antigen Binding Domains and Antigen Binding Domains with Conservative Substitutions

Variants of the antigen binding domains that bind CD20 are within the scope of the disclosure. For example, variants may comprise 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acid substitutions in the antigen binding domain that bind CD20 as long as they retain or have improved functional properties when compared to the parent antigen binding domains. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding domains that bind CD20 of the disclosure. In some embodiments, the variation is in the framework regions. In some embodiments, variants are generated by conservative substitutions.

Also provided are antigen binding domains that bind CD20 comprising the VH and the VL which are at least 80% identical to
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

In some embodiments, the identity is 85%. In some embodiments, the identity is 90%. In some embodiments, the identity is 91%. In some embodiments, the identity is 91%. In some embodiments, the identity is 92%. In some embodiments, the identity is 93%. In some embodiments, the identity is 94%. In some embodiments, the identity is 94%. In some embodiments, the identity is 95%. In some embodiments, the identity is 96%. In some embodiments, the identity is 97%. In some embodiments, the identity is 98%. In some embodiments, the identity is 99%.

The percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences.

The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at http_//_www gcg_com), using either a Blossum 62 matrix or a PAM250 matrix, and a gap weight of 16, 14, 12, 10, 8, 6, or 4 and a length weight of 1, 2, 3, 4, 5, or 6.

In some embodiments, variant antigen binding domains that bind CD20 comprise one or two conservative substitutions in any of the CDR regions, while retaining desired functional properties of the parent antigen binding fragments that bind CD20.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

Methods of Generating Antigen Binding Fragment that Bind CD20

Antigen binding domains that bind CD20 provided in the disclosure may be generated using various technologies. For example, the hybridoma method of Kohler and Milstein may be used to identify VH/VL pairs that bind CD20. In the hybridoma method, a mouse or other host animal, such as a hamster, rat or chicken is immunized with human and/or cyno CD20, followed by fusion of spleen cells from immunized animals with myeloma cells using standard methods to form hybridoma cells. Colonies arising from single immortalized hybridoma cells may be screened for production of the antibodies containing the antigen binding domains that bind CD20 with desired properties, such as specificity of binding, cross-reactivity or lack thereof, affinity for the antigen, and any desired functionality.

Antigen binding domains that bind CD20 generated by immunizing non-human animals may be humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen biding domains may be further optimized to improve their selectivity or affinity to a desired antigen by incorporating altered framework support residues to preserve binding affinity (backmutations) by techniques such as those described in Int. Patent Publ. Nos. WO1990/007861 and WO1992/22653, or by introducing variation at any of the CDRs for example to improve affinity of the antigen binding domain.

Transgenic animals, such as mice, rat or chicken carrying human immunoglobulin (Ig) loci in their genome may be used to generate antigen binding fragments that bind CD20, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc net), KyMab (http://_www_kymab_com), Trianni (http://_www.trianni_com) and Ablexis (http://_www_ablexis_com) may be engaged to provide human antibodies directed against a selected antigen using technologies as described above.

Antigen binding domains that bind CD20 may be selected from a phage display library, where the phage is engineered to express human immunoglobulins or portions thereof such as Fabs, single chain antibodies (scFv), or unpaired or paired antibody variable regions. The antigen binding domains that bind CD20 may be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries may be screened for phage binding to human and/or cyno CD20 and the obtained positive clones may be further characterized, the Fabs isolated from the clone lysates, and converted to scFvs or other configurations of antigen binding fragments.

Preparation of immunogenic antigens and expression and production of antigen binding domains of the disclosure may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

### Conjugation to Half-Life Extending Moieties

The antigen binding domains that bind CD20 of the disclosure may be conjugated to a half-life extending moiety. Exemplary half-life extending moieties are albumin, albumin variants, albumin-binding proteins and/or domains, transferrin and fragments and analogues thereof, immunoglobulins (Ig) or fragments thereof, such as Fc regions. Amino acid sequences of the aforementioned half-life extending moieties are known. Ig or fragments thereof include all isotypes, i.e., IgG1, IgG2, IgG3, IgG4, IgM, IgA and IgE.

Additional half-life extending moieties that may be conjugated to the antigen binding domains that bind CD20 of the disclosure include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the antigen binding domains that bind CD20 of the disclosure and may be generated by standard cloning and expression techniques. Alternatively, well known chemical coupling methods may be used to attach the moieties to recombinantly produced antigen binding domains that bind CD20 of the disclosure.

A pegyl moiety may for example be conjugated to the antigen binding domain that bind CD20 of the disclosure by incorporating a cysteine residue to the C-terminus of the antigen binding domain that bind CD20 of the disclosure, or engineering cysteines into residue positions that face away from the CD20 binding site and attaching a pegyl group to the cysteine using well known methods.

In some embodiments, the antigen binding fragment that binds CD20 is conjugated to a half-life extending moiety.

In some embodiments, the half-life extending moiety is an immunoglobulin (Ig), a fragment of the Ig, an Ig constant region, a fragment of the Ig constant region, a Fc region, transferrin, albumin, an albumin binding domain or polyethylene glycol. In some embodiments, the half-life extending moiety is an Ig constant region.

In some embodiments, the half-life extending moiety is the Ig.

In some embodiments, the half-life extending moiety is the fragment of the Ig.

In some embodiments, the half-life extending moiety is the Ig constant region.

In some embodiments, the half-life extending moiety is the fragment of the Ig constant region.

In some embodiments, the half-life extending moiety is the Fc region.

In some embodiments, the half-life extending moiety is albumin.

In some embodiments, the half-life extending moiety is the albumin binding domain.

In some embodiments, the half-life extending moiety is transferrin.

In some embodiments, the half-life extending moiety is polyethylene glycol.

The antigen binding domains that bind CD20 conjugated to a half-life extending moiety may be evaluated for their pharmacokinetic properties utilizing known in vivo models.

### Conjugation to Immunoglobulin (Ig) Constant Regions or Fragments of the Ig Constant Regions

The antigen binding domains that bind CD20 of the disclosure may be conjugated to an Ig constant region or a fragment of the Ig constant region to impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or the fragment of the Ig constant region functions also as a half-life extending moiety as discussed herein. The antigen binding domains that bind CD20 of the disclosure may be engineered into conventional full length antibodies using standard methods. The full length antibodies comprising the antigen binding domain that binds CD20 may further be engineered as described herein.

Immunoglobulin heavy chain constant region comprised of subdomains CH1, hinge, CH2 and CH3. The CH1 domain spans residues A118-V215, the CH2 domain residues A231-K340 and the CH3 domain residues G341-K447 on the heavy chain, residue numbering according to the EU Index. In some instances G341 is referred as a CH2 domain residue. Hinge is generally defined as including E216 and terminating at P230 of human IgG1. Ig Fc region comprises at least the CH2 and the CH3 domains of the Ig constant region, and therefore comprises at least a region from about A231 to K447 of Ig heavy chain constant region.

The invention also provides an antigen binding domain that binds CD20 conjugated to an immunoglobulin (Ig) constant region or a fragment of the Ig constant region.

In some embodiments, the Ig constant region is a heavy chain constant region

In some embodiments, the Ig constant region is a light chain constant region.

In some embodiments, the fragment of the Ig constant region comprises a Fc region.

In some embodiments, the fragment of the Ig constant region comprises a CH2 domain.

In some embodiments, the fragment of the Ig constant region comprises a CH3 domain.

In some embodiments, the fragment of the Ig constant region comprises the CH2 domain and the CH3 domain.

In some embodiments, the fragment of the Ig constant region comprises at least portion of a hinge, the CH2 domain and the CH3 domain. Portion of the hinge refers to one or more amino acid residues of the Ig hinge.

In some embodiments, the fragment of the Ig constant region comprises the hinge, the CH2 domain and the CH3 domain.

In some embodiments, the antigen binding domain that binds CD20 is conjugated to the N-terminus of the Ig constant region or the fragment of the Ig constant region.

In some embodiments, the antigen binding domain that binds CD20 is conjugated to the C-terminus of the Ig constant region or the fragment of the Ig constant region.

In some embodiments, the antigen binding domain that binds CD20 is conjugated to the Ig constant region or the fragment of the Ig constant region via a second linker (L2).

In some embodiments, the L2 comprises the amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61.

In some embodiments, the L2 comprises the amino acid sequence of SEQ ID NO: 28.

In some embodiments, the L2 comprises the amino acid sequence of SEQ ID NO: 29.

In some embodiments, the L2 comprises the amino acid sequence of SEQ ID NO: 30.

In some embodiments, the L2 comprises the amino acid sequence of SEQ ID NO: 31.

In some embodiments, the L2 comprises the amino acid sequence of SEQ ID NO: 32.

In some embodiments, the L2 comprises the amino acid sequence of SEQ ID NO: 33.

In some embodiments, the L2 comprises the amino acid sequence of SEQ ID NO: 34.

In some embodiments, the L2 comprises the amino acid sequence of SEQ ID NO: 35.

In some embodiments, the L2 comprises the amino acid sequence of SEQ ID NO: 36.

In some embodiments, the L2 comprises the amino acid sequence of SEQ ID NO: 37.

In some embodiments, the L2 comprises the amino acid sequence of SEQ ID NO: 38.

In some embodiments, the L2 comprises the amino acid sequence of SEQ ID NO: 39.

In some embodiments, the L2 comprises the amino acid sequence of SEQ ID NO: 40.

In some embodiments, the L2 comprises the amino acid sequence of SEQ ID NO: 41.

In some embodiments, the L2 comprises the amino acid sequence of SEQ ID NO: 42.

In some embodiments, the L2 comprises the amino acid sequence of SEQ ID NO: 43.

In some embodiments, the L2 comprises the amino acid sequence of SEQ ID NO: 44.

In some embodiments, the L2 comprises the amino acid sequence of SEQ ID NO: 45.

In some embodiments, the L2 comprises the amino acid sequence of SEQ ID NO: 46.

In some embodiments, the L2 comprises the amino acid sequence of SEQ ID NO: 47.

In some embodiments, the L2 comprises the amino acid sequence of SEQ ID NO: 48.

In some embodiments, the L2 comprises the amino acid sequence of SEQ ID NO: 49.

In some embodiments, the L2 comprises the amino acid sequence of SEQ ID NO: 50.

In some embodiments, the L2 comprises the amino acid sequence of SEQ ID NO: 51.

In some embodiments, the L2 comprises the amino acid sequence of SEQ ID NO: 52.

In some embodiments, the L2 comprises the amino acid sequence of SEQ ID NO: 53.

In some embodiments, the L2 comprises the amino acid sequence of SEQ ID NO: 54.

In some embodiments, the L2 comprises the amino acid sequence of SEQ ID NO: 55.

In some embodiments, the L2 comprises the amino acid sequence of SEQ ID NO: 56.

In some embodiments, the L2 comprises the amino acid sequence of SEQ ID NO: 57.

In some embodiments, the L2 comprises the amino acid sequence of SEQ ID NO: 58.

In some embodiments, the L2 comprises the amino acid sequence of SEQ ID NO: 59.

In some embodiments, the L2 comprises the amino acid sequence of SEQ ID NO: 60.

In some embodiments, the L2 comprises the amino acid sequence of SEQ ID NO: 61.

The antigen binding domains that bind CD20 of the disclosure conjugated to Ig constant region or the fragment of the Ig constant region may be assessed for their functionality using several known assays. Binding to CD20 may be assessed using methods described herein. Altered properties imparted by the Ig constant domain or the fragment of the Ig constant region such as Fc region may be assayed in Fc receptor binding assays using soluble forms of the receptors, such as the FcγRI, FcγRII, FcγRIII or FcRn receptors, or using cell-based assays measuring for example ADCC, CDC or ADCP.

ADCC may be assessed using an in vitro assay using CD20 expressing cells as target cells and NK cells as effector cells. Cytolysis may be detected by the release of label (e.g. radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

ADCP may be evaluated by using monocyte-derived macrophages as effector cells and any CD20 expressing cells as target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD 11⁺CD14⁺ macrophages using standard methods.

CDC of cells may be measured for example by plating Daudi cells at 1×10⁵ cells/well (50 L/well) in RPMI-B (RPMI supplemented with 1% BSA), adding 50 μL of test protein to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

Proteins Comprising the Antigen Binding Domains that Bind CD20 of the Disclosure

The antigen binding domains that bind CD20 of the disclosure may be engineered into monospecific or multispecific proteins of various designs using standard methods.

The disclosure also provides a monospecific protein comprising the antigen binding domain that binds CD20 of the disclosure.

In some embodiments, the monospecific protein is an antibody.

The disclosure also provides a multispecific protein comprising the antigen binding domain that binds CD20 of the disclosure.

In some embodiments, the multispecific protein is bispecific.

In some embodiments, the multispecific protein is trispecific.

In some embodiments, the multispecific protein is tetraspecific.

In some embodiments, the multispecific protein is monovalent for binding to CD20.

In some embodiments, the multispecific protein is bivalent for binding to CD20.

The disclosure also provides an isolated multispecific protein comprising a first antigen binding domain that binds CD20 and a second antigen binding domain that binds a lymphocyte antigen. In some embodiments, the second antigen binding domain that binds a lymphocyte antigen comprises a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14; a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18; a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22; or a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26.

In some embodiments, the lymphocyte antigen is a T cell antigen.

In some embodiments, the T cell antigen is a CD8⁺ T cell antigen.

In some embodiments, the lymphocyte antigen is a NK cell antigen.

In some embodiments, the lymphocyte antigen is CD3, CD3 epsilon (CD3ε), CD8, KI2L4, NKG2E, NKG2D, NKG2F, BTNL3, CD186, BTNL8, PD-1, CD195, or NKG2C.

In some embodiments, the lymphocyte antigen is CD3ε.

In some embodiments, the first antigen binding domain that binds CD20 and/or the second antigen binding domain that binds the lymphocyte antigen comprise a scFv, a (scFv)₂, a Fv, a Fab, a F(ab′)₂, a Fd, a dAb or a VHH.

In some embodiments, the first antigen binding domain that binds CD20 and/or the second antigen binding domain that binds the lymphocyte antigen comprise the Fab.

In some embodiments, the first antigen binding domain that binds CD20 and/or the second antigen binding domain that binds the lymphocyte antigen comprise the F(ab′)₂.

In some embodiments, the first antigen binding domain that binds CD20 and/or the second antigen binding domain that binds the lymphocyte antigen comprise the VHH.

In some embodiments, the first antigen binding domain that binds CD20 and/or the second antigen binding domain that binds the lymphocyte antigen comprise the Fv.

In some embodiments, the first antigen binding domain that binds CD20 and/or the second antigen binding domain that binds the lymphocyte antigen comprise the Fd.

In some embodiments, the first antigen binding domain that binds CD20 and/or the second antigen binding domain that binds the lymphocyte antigen comprise the scFv.

In some embodiments, the scFv comprises, from the N- to C-terminus, a VH, a first linker (L1) and a VL (VH-L1-VL) or the VL, the L1 and the VH (VL-L1-VH).

In some embodiments, the L1 comprises about 5-50 amino acids.

In some embodiments, the L1 comprises about 5-40 amino acids.

In some embodiments, the L1 comprises about 10-30 amino acids.

In some embodiments, the L1 comprises about 10-20 amino acids.

In some embodiments, the L1 comprises the amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61.

In some embodiments, the L1 comprises the amino acid sequence of SEQ ID NO: 28.

In some embodiments, the L1 comprises the amino acid sequence of SEQ ID NO: 29.

In some embodiments, the L1 comprises the amino acid sequence of SEQ ID NO: 30.

In some embodiments, the L1 comprises the amino acid sequence of SEQ ID NO: 31.

In some embodiments, the L1 comprises the amino acid sequence of SEQ ID NO: 32.

In some embodiments, the L1 comprises the amino acid sequence of SEQ ID NO: 33.

In some embodiments, the L1 comprises the amino acid sequence of SEQ ID NO: 34.

In some embodiments, the L1 comprises the amino acid sequence of SEQ ID NO: 35.

In some embodiments, the L1 comprises the amino acid sequence of SEQ ID NO: 36.

In some embodiments, the L1 comprises the amino acid sequence of SEQ ID NO: 37.

In some embodiments, the L1 comprises the amino acid sequence of SEQ ID NO: 38.

In some embodiments, the L1 comprises the amino acid sequence of SEQ ID NO: 39.

In some embodiments, the L1 comprises the amino acid sequence of SEQ ID NO: 40.

In some embodiments, the L1 comprises the amino acid sequence of SEQ ID NO: 41.

In some embodiments, the L1 comprises the amino acid sequence of SEQ ID NO: 42.

In some embodiments, the L1 comprises the amino acid sequence of SEQ ID NO: 43.

In some embodiments, the L1 comprises the amino acid sequence of SEQ ID NO: 44.

In some embodiments, the L1 comprises the amino acid sequence of SEQ ID NO: 45.

In some embodiments, the L1 comprises the amino acid sequence of SEQ ID NO: 46.

In some embodiments, the L1 comprises the amino acid sequence of SEQ ID NO: 47.

In some embodiments, the L1 comprises the amino acid sequence of SEQ ID NO: 48.

In some embodiments, the L1 comprises the amino acid sequence of SEQ ID NO: 49.

In some embodiments, the L1 comprises the amino acid sequence of SEQ ID NO: 50.

In some embodiments, the L1 comprises the amino acid sequence of SEQ ID NO: 51.

In some embodiments, the L1 comprises the amino acid sequence of SEQ ID NO: 52.

In some embodiments, the L1 comprises the amino acid sequence of SEQ ID NO: 53.

In some embodiments, the L1 comprises the amino acid sequence of SEQ ID NO: 54.

In some embodiments, the L1 comprises the amino acid sequence of SEQ ID NO: 55.

In some embodiments, the L1 comprises the amino acid sequence of SEQ ID NO: 56.

In some embodiments, the L1 comprises the amino acid sequence of SEQ ID NO: 57.

In some embodiments, the L1 comprises the amino acid sequence of SEQ ID NO: 58.

In some embodiments, the L1 comprises the amino acid sequence of SEQ ID NO: 59.

In some embodiments, the L1 comprises the amino acid sequence of SEQ ID NO: 60.

In some embodiments, the L1 comprises the amino acid sequence of SEQ ID NO: 61.

In some embodiments, the first antigen binding domain that binds CD20 comprises the HCDR1 of SEQ ID NOs: 1 or 7, the HCDR2 of SEQ ID NOs: 2 or 8, the HCDR3 of SEQ ID NOs: 3, 9 or 64, the LCDR1 of SEQ ID NOs: 4, 10 or 65, the LCDR2 of SEQ ID NO: 5, and the LCDR3 of SEQ ID NOs: 6 or 11.

In some embodiments, the first antigen binding domain that binds CD20 comprises the HCDR1, the HCDR2, the HCDR3, the LCDR1, the LCDR2 and the LCDR3 of
SEQ ID NOs: 1, 2, 3, 4, 5 and 6, respectively;
SEQ ID NOs: 7, 8, 9, 10, 5 and 11, respectively;
SEQ ID NOs: 1, 2, 64, 65, 5 and 11, respectively; or
SEQ ID NOs: 7, 2, 9, 10, 5 and 11, respectively.

In some embodiments, the first antigen binding domain that binds CD20 comprises the VH of SEQ ID NO: 12 and the VL of SEQ ID NO: 14.

In some embodiments, the first antigen binding domain that binds CD20 comprises the VH of SEQ ID NO: 16 and the VL of SEQ ID NO: 18.

In some embodiments, the first antigen binding domain that binds CD20 comprises the VH of SEQ ID NO: 20 and the VL of SEQ ID NO: 22.

In some embodiments, the first antigen binding domain that binds CD20 comprises the VH of SEQ ID NO: 24 and the VL of SEQ ID NO: 26.

In some embodiments, the first antigen binding domain that binds CD20 comprises the VH of SEQ ID NOs: 12, 16, 20, or 24 and the VL of SEQ ID NOs: 14, 18, 22 or 26.

In some embodiments, the first antigen binding domain that binds CD20 comprises:
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

In some embodiments, the first antigen binding domain that binds CD20 is conjugated to a first immunoglobulin (Ig) constant region or a fragment of the first Ig constant region and/or the second antigen binding domain that binds the lymphocyte antigen is conjugated to a second immunoglobulin (Ig) constant region or a fragment of the second Ig constant region.

In some embodiments, the fragment of the first Ig constant region and/or the fragment of the second Ig constant region comprises a Fc region.

In some embodiments, the fragment of the first Ig constant region and/or the fragment of the second Ig constant region comprises a CH2 domain.

In some embodiments, the fragment of the first Ig constant region and/or the fragment of the second Ig constant region comprises a CH3 domain.

In some embodiments, the fragment of the first Ig constant region and/or the fragment of the second Ig constant region comprises the CH2 domain and the CH3 domain.

In some embodiments, the fragment of the first Ig constant region and/or the fragment of the second Ig constant region comprises at least portion of a hinge, the CH2 domain and the CH3 domain.

In some embodiments, the fragment of the Ig constant region comprises the hinge, the CH2 domain and the CH3 domain.

In some embodiments, the multispecific protein further comprises a second linker (L2) between the first antigen binding domain that binds CD20 and the first Ig constant region or the fragment of the first Ig constant region and the second antigen binding domain that binds the lymphocyte antigen and the second Ig constant region or the fragment of the second Ig constant region.

In some embodiments, the L2 comprises the amino acid sequence of SEQ ID NOs: 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or 61.

In some embodiments, the first Ig constant region or the fragment of the first Ig constant region and the second Ig constant region or the fragment of the second Ig constant region is an IgG1, an IgG2, and IgG3 or an IgG4 isotype.

In some embodiments, the first Ig constant region or the fragment of the first Ig constant region and the second Ig constant region or the fragment of the second Ig constant region is an IgG1 isotype.

In some embodiments, the first Ig constant region or the fragment of the first Ig constant region and the second Ig constant region or the fragment of the second Ig constant region is an IgG2 isotype.

In some embodiments, the first Ig constant region or the fragment of the first Ig constant region and the second Ig constant region or the fragment of the second Ig constant region is an IgG3 isotype.

In some embodiments, the first Ig constant region or the fragment of the first Ig constant region and the second Ig constant region or the fragment of the second Ig constant region is an IgG4 isotype.

The first Ig constant region or the fragment of the first Ig constant region and the second Ig constant region or the fragment of the second Ig constant region can further be engineered as described herein.

In some embodiments, the first Ig constant region or the fragment of the first Ig constant region and the second Ig constant region or the fragment of the second Ig constant region comprises at least one mutation that results in reduced binding of the multispecific protein to a FcγR.

In some embodiments, the at least one mutation that results in reduced binding of the multispecific protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

In some embodiments, the first Ig constant region or the fragment of the first Ig constant region and the second Ig constant region or the fragment of the second Ig constant region comprises at least one mutation that results in enhanced binding of the multispecific protein to a Fcγ receptor (FcγR).

In some embodiments, the at least one mutation that results in enhanced binding of the multispecific protein to the FcγR is selected from the group consisting of S239D/I332E, S298A/E333A/K334A, F243L/R292P/Y300L, F243L/R292P/Y300L/P396L, F243L/R292P/Y300L/V305I/P396L and G236A/S239D/I332E, wherein residue numbering is according to the EU index.

In some embodiments, the FcγR is FcγRI, FcγRIIA, FcγRIIB or FcγRIII, or any combination thereof.

In some embodiments, the first Ig constant region or the fragment of the first Ig constant region and the second Ig constant region or the fragment of the second Ig constant region comprises at least one mutation that modulates a half-life of the multispecific protein.

In some embodiments, the at least one mutation that modulates the half-life of the multispecific protein is selected from the group consisting of H435A, P257I/N434H, D376V/N434H, M252Y/S254T/T256E/H433K/N434F, T308P/N434A and H435R, wherein residue numbering is according to the EU index.

In some embodiments, the multispecific protein comprises at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region.

In some embodiments, the at least one mutation in a CH3 domain of the first Ig constant region or in a CH3 domain of the fragment of the first Ig constant region and/or at least one mutation in a CH3 domain of the second Ig constant region or in a CH3 domain of the fragment of the second Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

In some embodiments, the first Ig constant region or the fragment of the first Ig constant region and the second Ig constant region or the fragment of the second Ig constant region comprise the following mutations L235A_L235A_D265S_T350V_L351Y_F405A_Y407V in the first Ig constant region and L235A_L235A_D265S_T350V_T366L_K392L_T394W in the second Ig constant region; or L235A_L235A_D265S_T350V_T366L_K392L_T394W in the first Ig constant region and L235A_L235A_D265S_T350V_L351Y_F405A_Y407V in the second Ig constant region.

Generation of Multispecific Proteins that Comprise Antigen Binding Fragments that Bind CD20

The antigen binding fragments that bind CD20 of the disclosure may be engineered into multispecific antibodies which are also encompassed within the scope of the invention.

The antigen binding fragments that bind CD20 may be engineered into full length multispecific antibodies which are generated using Fab arm exchange, in which substitutions are introduced into two monospecific bivalent antibodies within the Ig constant region CH3 domain which promote Fab arm exchange in vitro. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g. Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I _K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chai heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the antigen binding domains that bind CD20 can be incorporated include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

The antigen binding domains that bind CD20 of the disclosure may also be engineered into multispecific proteins which comprise three polypeptide chains. In such designs, at least one antigen binding domain is in the form of a scFv. Exemplary designs include (in which “1” indicates the first antigen binding domain, “2” indicates the second antigen binding domain and “3” indicates the third antigen binding domain:
Design 1: Chain A) scFv1-CH2-CH3; Chain B) VL2-CL; Chain C) VH2-CH1-hinge-CH2-CH3
Design 2: Chain A) scFv1-hinge-CH2-CH3; Chain B) VL2-CL; Chain C) VH2-CH1-hinge-CH2-CH3
Design 3: Chain A) scFv1-CH1-hinge-CH2-CH3; Chain B) VL2-CL; Chain C) VH2-CH1-hinge-CH2-CH3
Design 4: Chain A) CH2-CH3-scFv1; Chain B) VL2-CL; Chain C) VH2-CH1-hinge-CH2-CH3

CH3 engineering may be incorporated to the Designs 1-4, such as mutations L351Y_F405A_Y407V/T394W, T366I _K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

### Isotypes, Allotypes and Fc Engineering

The Ig constant region or the fragment of the Ig constant region, such as the Fc region present in the proteins of the disclosure may be of any allotype or isotype.

In some embodiments, the Ig constant region or the fragment of the Ig constant region is an IgG1 isotype.

In some embodiments, the Ig constant region or the fragment of the Ig constant region is an IgG2 isotype.

In some embodiments, the Ig constant region or the fragment of the Ig constant region is an IgG3 isotype.

In some embodiments, the Ig constant region or the fragment of the Ig constant region is an IgG4 isotype.

The Ig constant region or the fragment of the Ig constant region may be of any allotype. It is expected that allotype has no influence on properties of the Ig constant region, such as binding or Fc-mediated effector functions. Immunogenicity of therapeutic proteins comprising Ig constant regions of fragments thereof is associated with increased risk of infusion reactions and decreased duration of therapeutic response (Baert et al., (2003) N. Engl. J Med. 348:602-08). The extent to which therapeutic proteins comprising Ig constant regions of fragments thereof induce an immune response in the host may be determined in part by the allotype of the Ig constant region (Stickler et al., (2011) Genes and Immunity 12:213-21). Ig constant region allotype is related to amino acid sequence variations at specific locations in the constant region sequences of the antibody. Table 3 shows select IgG1, IgG2 and IgG4 allotypes.

TABLE 3

| | Amino acid residue at position of diversity (residue numbering: EU Index) | | | | | | | |
|---|---|---|---|---|---|---|---|---|
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| Glm(17) | | | | | K | E | M | A |
| Glm(17, 1) | | | | | K | D | L | A |

C-terminal lysine (CTL) may be removed from the Ig constant region by endogenous circulating carboxypeptidases in the blood stream (Cai et al., (2011) Biotechnol. Bioeng. 108:404-412). During manufacturing, CTL removal may be controlled to less than the maximum level by control of concentration of extracellular Zn²⁺, EDTA or EDTA—Fe³⁺ as described in U.S. Patent Publ. No. US20140273092. CTL content of proteins may be measured using known methods.

In some embodiments, the antigen binding fragment that binds CD20 conjugated to the Ig constant region has a C-terminal lysine content from about 10% to about 90%. In some embodiments, the C-terminal lysine content is from about 20% to about 80%. In some embodiments, the C-terminal lysine content is from about 40% to about 70%. In some embodiments, the C-terminal lysine content is from about 55% to about 70%. In some embodiments, the C-terminal lysine content is about 60%.

Fc region mutations may be made to the antigen binding domains that bind CD20 conjugated to the Ig constant region or to the fragment of the Ig constant region to modulate their effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), inhibitory FcγRIIb and/or to FcRn.

In some embodiments, the antigen binding domain that binds CD20 conjugated to the Ig constant region or the fragment of the Ig constant region comprises at least one mutation in the Ig constant region or in the fragment of the Ig constant region.

In some embodiments, the at least one mutation is in the Fc region.

In some embodiments, the antigen binding domain that binds CD20 conjugated to the Ig constant region or to the fragment of the Ig constant region comprises at least one, two, three, four, five, six, seven, eight, nine, ten, eleven, twelve, thirteen, fourteen or fifteen mutations in the Fc region.

In some embodiments, the antigen binding domain that binds CD20 conjugated to the Ig constant region or to the fragment of the Ig constant region comprises at least one mutation in the Fc region that modulates binding of the antibody to FcRn.

Fc positions that may be mutated to modulate half-life (e.g. binding to FcRn) include positions 250, 252, 253, 254, 256, 257, 307, 376, 380, 428, 434 and 435. Exemplary mutations that may be made singularly or in combination are mutations T250Q, M252Y, I253A, S254T, T256E, P257I, T307A, D376V, E380A, M428L, H433K, N434S, N434A, N434H, N434F, H435A and H435R. Exemplary singular or combination mutations that may be made to increase the half-life are mutations M428L/N434S, M252Y/S254T/T256E, T250Q/M428L, N434A and T307A/E380A/N434A. Exemplary singular or combination mutations that may be made to reduce the half-life are mutations H435A, P257I/N434H, D376V/N434H, M252Y/S254T/T256E/H433K/N434F, T308P/N434A and H435R.

In some embodiments, the antigen binding domain that binds CD20 conjugated to the Ig constant region or to the fragment of the Ig constant region comprises M252Y/S254T/T256E mutation.

In some embodiments, the antigen binding domain that binds CD20 conjugated to the Ig constant region or to the fragment of the Ig constant region comprises at least one mutation in the Fc region that reduces binding of the protein to an activating Fey receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding of the protein to the activating FcγR and subsequently to reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in proteins with reduced ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

An exemplary mutation that result in proteins with reduced CDC is a K322A mutation.

Well-known S228P mutation may be made in IgG4 to enhance IgG4 stability.

In some embodiments, the antigen binding domain that binds CD20 conjugated to the Ig constant region or to the fragment of the Ig constant region comprises at least one mutation selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, K322, A330S and P331S.

In some embodiments, the antigen binding domain that binds CD20 conjugated to the Ig constant region or to the fragment of the Ig constant region comprises L234A/L235A/D265S mutation.

In some embodiments, the antigen binding domain that binds CD20 conjugated to the Ig constant region or to the fragment of the Ig constant region comprises L234A/L235A mutation.

In some embodiments, the antigen binding domain that binds CD20 conjugated to the Ig constant region or to the fragment of the Ig constant region comprises at least one mutation in the Fc region that enhances binding of the protein to an Fcγ receptor (FcγR) and/or enhances Fc effector functions such as C1q binding, complement dependent cytotoxicity (CDC), antibody-dependent cell-mediated cytotoxicity (ADCC) and/or phagocytosis (ADCP).

Fc positions that may be mutated to increase binding of the protein to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index). Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination mutations that result in proteins with increased ADCC or ADCP are a S239D/I332E, S298A/E333A/K334A, F243L/R292P/Y300L, F243L/R292P/Y300L/P396L, F243L/R292P/Y300L/V305I/P396L and G236A/S239D/I332E.

Fc positions that may be mutated to enhance CDC include positions 267, 268, 324, 326, 333, 345 and 430. Exemplary mutations that may be made singularly or in combination are S267E, F1268F, S324T, K326A, K326W, E333A, E345K, E345Q, E345R, E345Y, E430S, E430F and E430T. Exemplary combination mutations that result in proteins with increased CDC are K326A/E333A, K326W/E333A, H268F/S324T, S267E/H268F, S267E/S324T and S267E/H268F/S324T.

The specific mutations described herein are mutations when compared to the IgG1 and IgG4 wild-type amino acid sequences of SEQ ID NOs: 62, and 63, respectively.

SEQ ID NO: 62, wild-type IgG1
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 63; wild-type IgG4
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

Binding of the antibody to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test antibody on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

### Glycoengineering

The ability of the antigen binding domain that binds CD20 conjugated to the Ig constant region or to the fragment of the Ig constant region to mediate ADCC can be enhanced by engineering the Ig constant region or the fragment of the Ig constant region oligosaccharide component. Human IgG1 or IgG3 are N-glycosylated at Asn297 with the majority of the glycans in the well-known biantennary G0, G0F, G1, G1F, G2 or G2F forms. Ig constant region containing proteins may be produced by non-engineered CHO cells typically have a glycan fucose content of about at least 85%. The removal of the core fucose from the biantennary complex-type oligosaccharides attached to the antigen binding domain that binds CD20 conjugated to the Ig constant region or to the fragment of the Ig constant region enhances the ADCC of the protein via improved FcγRIIIa binding without altering antigen binding or CDC activity. Such proteins can be achieved using different methods reported to lead to the successful expression of relatively high defucosylated immunoglobulins bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4): 405-415, 2010; PMID:20562582), application of a rat hybridoma cell line YB2/0 as the host cell line (Shinkawa et al., J Biol Chem 278:3466-3473, 2003), introduction of small interfering RNA specifically against the a 1,6-fucosyltrasferase (FUT8) gene (Mori et al., Biotechnol Bioeng 88:901-908, 2004), or coexpression of 0-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments, the antigen binding domain that binds CD20 conjugated to the Ig constant region or to the fragment of the Ig constant region of the disclosure has a biantennary glycan structure with fucose content of about between 1% to about 15%, for example about 15%, 14%, 13%, 12%, 11% 10%, 9% 8, 7%, 6%, 5%, 4%, 3%, 2% or 1%. In some embodiments, the antigen binding domain that binds CD20 conjugated to the Ig constant region or to the fragment of the Ig constant region has a glycan structure with fucose content of about 50%, 40%, 45%, 40%, 35%, 30%, 25%, or 20%.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Int Pat. Publ. No. WO2008/077546 2); 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to the antigen binding domain that bind CD20 conjugated to the Ig constant region or to the fragment of the Ig constant region with fucose content of about between 1%-15%.

“Normal fucose” or “normal fucose content” as used herein refers to the antigen binding domain that bind CD20 conjugated to the Ig constant region or to the fragment of the Ig constant region with fucose content of about over 50%, typically about over 80% or over 85%.

### Anti-Idiotypic Antibodies

Anti-idiotypic antibodies are antibodies that specifically bind to the antigen binding domain that binds CD20 of the disclosure.

The invention also provides an anti-idiotypic antibody that specifically binds to the antigen binding domain that binds CD20 of the disclosure.

The invention also provides an anti-idiotypic antibody that specifically binds to the antigen binding domain that binds CD20 comprising
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

An anti-idiotypic (Id) antibody is an antibody which recognizes the antigenic determinants (e.g. the paratope or CDRs) of the antibody. The Id antibody may be antigen-blocking or non-blocking. The antigen-blocking Id may be used to detect the free antigen binding domain in a sample (e.g. the antigen binding domain that binds CD20 of the disclosure). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original antigen binding domain which induced the anti-Id. Thus, by using antibodies to the idiotypic determinants of the antigen binding domain, it is possible to identify other clones expressing antigen binding domains of identical specificity. Anti-Id antibodies may be varied (thereby producing anti-Id antibody variants) and/or derivatized by any suitable technique, such as those described elsewhere herein.

### Immunoconjugates

The antigen binding domains that bind CD20 of the disclosure, the proteins comprising the antigen binding domains that bind CD20 or the multispecific proteins that comprise the antigen binding domains that bind CD20 (collectively referred herein as to CD20 binding proteins) may be conjugated to a heterologous molecule.

In some embodiments, the heterologous molecule is a detectable label or a cytotoxic agent.

The invention also provides an antigen binding domain that binds CD20 conjugated to a detectable label.

The invention also provides a protein comprising an antigen binding domain that binds CD20 conjugated to a detectable label.

The invention also provides a multispecific protein comprising an antigen binding domain that binds CD20 conjugated to a detectable label.

The invention also provides an antigen binding domain that binds CD20 conjugated to a cytotoxic agent.

The invention also provides a protein comprising an antigen binding domain that binds CD20 conjugated to a cytotoxic agent.

The invention also provides a multispecific protein comprising an antigen binding domain that binds CD20 conjugated to a cytotoxic agent.

CD20 binding proteins of the disclosure may be used to direct therapeutics to CD20 expressing cells, such as B-cell lymphoma, non-Hodgkin lymphoma, diffuse large B-cell lymphoma (DLBCL), a mantle cell lymphoma (MCL), a follicular lymphoma (FL), or acute lymphoblastic leukemia (ALL) cells. Alternatively, CD20 expressing cells may be targeted with a CD20 binding protein of the disclosure coupled to a therapeutic intended to modify cell function once internalized.

In some embodiments, the detectable label is also a cytotoxic agent.

The CD20 binding proteins of the disclosure conjugated to a detectable label may be used to evaluate expression of CD20 on a variety of samples.

Detectable label includes compositions that when conjugated to the CD20 binding proteins of the disclosure renders the latter detectable, via spectroscopic, photochemical, biochemical, immunochemical, or chemical means.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e. iodine) to 83 (i.e. bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The antigen binding domain that binds CD20 conjugated to a detectable label may be used as an imaging agent.

The protein comprising an antigen binding domain that binds CD20 conjugated to a detectable label may be used as an imaging agent.

The multispecific protein comprising an antigen binding domain that binds CD20 conjugated to a detectable label may be used as an imaging agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y and ¹⁸⁶Re.

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the antibody of the invention through the N (amino) terminus or the C (carboxyl) terminus of the peptidic drug moiety (WO02/088172), or via any cysteine engineered into the antibody.

The CD20 binding proteins of the disclosure may be conjugated to a detectable label using known methods.

In some embodiments, the detectable label is complexed with a chelating agent.

In some embodiments, the detectable label is conjugated to the CD20 binding proteins of the disclosure via a linker.

The detectable label or the cytotoxic moiety may be linked directly, or indirectly, to the CD20 binding proteins of the disclosure using known methods. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10, tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

In some embodiments, the CD20 binding proteins of the disclosure is removed from the blood via renal clearance.

### Kits

The invention also provides a kit comprising the antigen binding domain that binds CD20.

The invention also provides a kit comprising the protein comprising an antigen binding domain that binds CD20.

The invention also provides a kit comprising the multispecific protein comprising an antigen binding domain that binds CD20.

The kit may be used for therapeutic uses and as diagnostic kits.

The kit may be used to detect the presence of CD20 in a sample.

In some embodiments, the kit comprises the CD20 binding protein of the disclosure and reagents for detecting the CD20 binding protein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antigen binding domain that binds CD20 in a container and instructions for use of the kit.

In some embodiments, the kit comprises the protein comprising an antigen binding domain that binds CD20 in a container and instructions for use of the kit.

In some embodiments, the kit comprises the multispecific protein comprising an antigen binding domain that binds CD20 in a container and instructions for use of the kit.

In some embodiments, the antigen binding domain that binds CD20 in the kit is labeled.

In some embodiments, the protein comprising an antigen binding domain that binds CD20 in the kit is labeled.

In some embodiments, the multispecific protein comprising an antigen binding domain that binds CD20 in the kit is labeled.

In some embodiments, the kit comprises the antigen binding domain that binds CD20 comprising
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

### Methods of Detecting CD20

The invention also provides a method of detecting CD20 in a sample, comprising obtaining the sample, contacting the sample with the antigen binding domain that binds CD20 of the disclosure and detecting the bound CD20 in the sample.

In some embodiments, the sample may be derived from urine, blood, serum, plasma, saliva, ascites, circulating cells, synovial fluid, circulating cells, cells that are not tissue associated (i.e., free cells), tissues (e.g., surgically resected tissue, biopsies, including fine needle aspiration), histological preparations, and the like.

The antigen binding domain that binds CD20 of the disclosure may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antigen binding domain that binds CD20 of the disclosure may be used in a variety of assays to detect CD20 in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

### Polynucleotides, Host Cells and Vectors

The disclosure also provides an isolated polynucleotide encoding any of the CD20 binding proteins of the disclosure. The CD20 binding protein includes the antigen binding domains that bind CD20, the proteins comprising the antigen binding domains that bind CD20, the multispecific proteins that comprise the antigen binding domains that bind CD20 and the chimeric antigen receptors (CAR) comprising the antigen binding domains that bind CD20 of the disclosure.

The invention also provides an isolated polynucleotide encoding any of CD20 biding proteins or fragments thereof.

The invention also provides an isolated polynucleotide encoding the VH of SEQ ID NO: 12.

The invention also provides an isolated polynucleotide encoding the VH of SEQ ID NO: 16.

The invention also provides an isolated polynucleotide encoding the VH of SEQ ID NO: 20.

The invention also provides an isolated polynucleotide encoding the VH of SEQ ID NO: 24.

The invention also provides an isolated polynucleotide encoding the VL of SEQ ID NO: 14.

The invention also provides an isolated polynucleotide encoding the VL of SEQ ID NO: 18.

The invention also provides an isolated polynucleotide encoding the VL of SEQ ID NO: 22.

The invention also provides an isolated polynucleotide encoding the VL of SEQ ID NO: 26.

The invention also provides for an isolated polynucleotide encoding
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

Some embodiments of the disclosure also provide an isolated or purified nucleic acid comprising a polynucleotide which is complementary to the polynucleotides encoding the CD20 binding proteins of the disclosure or polynucleotides which hybridize under stringent conditions to the polynucleotides encoding the CD20 binding proteins of the disclosure.

The polynucleotides which hybridize under stringent conditions may hybridize under high stringency conditions. By “high stringency conditions” is meant that the polynucleotide specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

The polynucleotide sequences of the disclosure may be operably linked to one or more regulatory elements, such as a promoter or enhancer, that allow expression of the nucleotide sequence in the intended host cell. The polynucleotide may be a cDNA. The promoter bay be a strong, weak, tissue-specific, inducible or developmental-specific promoter. Exemplary promoters that may be used are hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. Inducible promoters such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like may also be used.

The invention also provides a vector comprising the polynucleotide of the invention. The disclosure also provide an expression vector comprising the polynucleotide of the invention. Such vectors may be plasmid vectors, viral vectors, vectors for baculovirus expression, transposon based vectors or any other vector suitable for introduction of the synthetic polynucleotide of the invention into a given organism or genetic background by any means. Polynucleotides encoding the CD20 binding proteins of the disclosure may be operably linked to control sequences in the expression vector(s) that ensure the expression of the CD20 binding proteins. Such regulatory elements may include a transcriptional promoter, sequences encoding suitable mRNA ribosomal binding sites, and sequences that control the termination of transcription and translation. Expression vectors may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The expression vectors can comprise naturally-occurring or non-naturally-occurring internucleotide linkages, or both types of linkages. The non-naturally occurring or altered nucleotides or internucleotide linkages do not hinder the transcription or replication of the vector.

Once the vector has been incorporated into the appropriate host, the host is maintained under conditions suitable for high level expression of the CD20 binding proteins of the disclosure encoded by the incorporated polynucleotides. The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

Vectors of the Disclosure May Also Contain One or More Internal Ribosome Entry Site(s) (IRES). Inclusion of an IRES sequence into fusion vectors may be beneficial for enhancing expression of some proteins. In some embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol x resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Exemplary vectors that may be used are Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif), the pET series (Novagen, Madison, Wis.), the pGEX series (Pharmacia Biotech, Uppsala, Sweden), and the pEX series (Clontech, Palo Alto, Calif). Bacteriophage vectors, such as λGT10, λGT11, λEMBL4, and λNM1149, λZapII (Stratagene) can be used. Exemplary plant expression vectors include pBI01, pBI01.2, pBI121, pBI101.3, and pBIN19 (Clontech). Exemplary animal expression vectors include pEUK-C1, pMAM, and pMAMneo (Clontech). The expression vector may be a viral vector, e.g., a retroviral vector, e.g., a gamma retroviral vector.

In some embodiments, the vector comprises the polynucleotide encoding the VH of SEQ ID NO: 12.

In some embodiments, the vector comprises the polynucleotide encoding the VH of SEQ ID NO: 16.

In some embodiments, the vector comprises the polynucleotide encoding the VH of SEQ ID NO: 20.

In some embodiments, the vector comprises the polynucleotide encoding the VH of SEQ ID NO: 24.

In some embodiments, the vector comprises the polynucleotide encoding the VL of SEQ ID NO: 14.

In some embodiments, the vector comprises the polynucleotide encoding the VL of SEQ ID NO: 18.

In some embodiments, the vector comprises the polynucleotide encoding the VL of SEQ ID NO: 22.

In some embodiments, the vector comprises the polynucleotide encoding the VL of SEQ ID NO: 26.

In some embodiments, the vector comprises the polynucleotide encoding
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

Embodiments of the invention further provide host cells comprising any of the recombinant expression vectors described herein. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, polypeptide, or protein, the host cell may be a mammalian cell. The host cell may be a human cell. While the host cell can be of any cell type, can originate from any type of tissue, and can be of any developmental stage, the host cell may be a peripheral blood lymphocyte (PBL). The host cell may be a T cell.

Also provided are a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, an erythrocyte, a neutrophil, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment, the population of cells is a clonal population comprising host cells comprising a recombinant expression vector as described herein.

The disclosure also provides a method of producing the CD20 binding protein of the disclosure comprising culturing the host cell of the disclosure in conditions that the CD20 binding protein is expressed, and recovering the CD20 binding protein produced by the host cell. Methods of making proteins and purifying them are known. Once synthesized (either chemically or recombinantly), the CD20 binding proteins may be purified according to standard procedures, including ammonium sulfate precipitation, affinity columns, column chromatography, high performance liquid chromatography (HPLC) purification, gel electrophoresis, and the like (see generally Scopes, Protein Purification (Springer—Verlag, N.Y., (1982)). A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

The polynucleotides encoding the CD20 binding proteins of the disclosure may be incorporated into vectors using standard molecular biology methods. Host cell transformation, culture, antibody expression and purification are done using well known methods.

### Pharmaceutical Compositions/Administration

The disclosure also provides a pharmaceutical composition comprising the CD20 binding protein of the disclosure and a pharmaceutically acceptable carrier.

The disclosure also provides a pharmaceutical composition comprising the antigen binding domain that binds CD20 of the disclosure and a pharmaceutically acceptable carrier.

The disclosure also provides a pharmaceutical composition comprising the protein comprising the antigen binding domain that binds CD20 of the disclosure and a pharmaceutically acceptable carrier.

The disclosure also provides a pharmaceutical composition comprising the multispecific protein comprising the antigen binding domain that binds CD20 of the disclosure and a pharmaceutically acceptable carrier.

The disclosure also provides a pharmaceutical composition comprising the antigen binding domain that binds CD20 of the disclosure and a pharmaceutically acceptable carrier.

For therapeutic use, the CD20 binding protein of the disclosure may be prepared as pharmaceutical compositions containing an effective amount of the antibody as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the CD79-binding proteins of the invention in such pharmaceutical formulation may vary, from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

The mode of administration of the CD20 binding protein of the disclosure may be any suitable route such as parenteral administration, e.g., intradermal, intramuscular, intraperitoneal, intravenous or subcutaneous, transmucosal (oral, intranasal, intravaginal, rectal) or other means appreciated by the skilled artisan, as well known in the art.

The CD20 binding protein of the disclosure of the invention may also be administered prophylactically in order to reduce the risk of developing a disease such as cancer.

Thus, a pharmaceutical composition of the invention for intramuscular injection may be prepared to contain 1 ml sterile buffered water, and between about 1 ng to about 100 mg/kg, e.g. about 50 ng to about 30 mg/kg or more preferably, about 5 mg to about 25 mg/kg, of the CD20 binding protein of the disclosure of the invention.

In embodiments of the present disclosure, the CD20 binding protein-expressing cells may be provided in compositions, e.g., suitable pharmaceutical composition(s) comprising the CD20 binding protein-expressing cells and a pharmaceutically acceptable carrier. In one aspect, the present disclosure provides pharmaceutical compositions comprising an effective amount of a lymphocyte expressing one or more of the CD20 binding proteins described and a pharmaceutically acceptable excipient. Pharmaceutical compositions of the present disclosure may comprise a CD20 binding protein-expressing cell, e.g., a plurality of CD20 binding protein-expressing cells, as described herein, in combination with one or more pharmaceutically or physiologically acceptable carriers, excipients or diluents. A pharmaceutically acceptable carrier can be an ingredient in a pharmaceutical composition, other than an active ingredient, which is nontoxic to the subject.

A pharmaceutically acceptable carrier can include a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Pharmaceutical compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Pharmaceutical compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

### Methods of Treatment and Uses

The disclosure also provides a method of treating a CD20 expressing cancer in a subject, comprising administering a therapeutically effective amount of the antigen biding domain that binds CD20 of the disclosure to the subject in need thereof for a time sufficient to treat the CD20 expressing cancer.

The disclosure also provides a method of treating a CD20 expressing cancer in a subject, comprising administering a therapeutically effective amount of the protein comprising the antigen biding domain that binds CD20 of the disclosure to the subject for a time sufficient to treat the CD20 expressing cancer

The disclosure also provides a method of treating a CD20 expressing cancer in a subject, comprising administering a therapeutically effective amount of the multispecific protein comprising the antigen biding domain that binds CD20 of the disclosure to the subject for a time sufficient to treat the CD20 expressing cancer.

The disclosure also provides a method of treating a CD20 expressing cancer in a subject, comprising administering a therapeutically effective amount of the antigen biding domain that binds CD20 of the disclosure to the subject for a time sufficient to treat the CD20 expressing cancer.

The disclosure also provides a method of treating a CD20 expressing cancer in a subject, comprising administering a therapeutically effective amount of the immunoconjugate comprising the antigen biding domain that binds CD20 of the disclosure to the subject for a time sufficient to treat the CD20 expressing cancer.

The disclosure also provides a method of treating a CD20 expressing cancer in a subject, comprising administering a therapeutically effective amount of the pharmaceutical composition comprising the antigen biding domain that binds CD20 of the disclosure to the subject for a time sufficient to treat the CD20 expressing cancer.

In one aspect, the disclosure relates generally to the treatment of a subject at risk of developing cancer. The invention also includes treating a malignancy or an autoimmune disease in which chemotherapy and/or immunotherapy results in significant immunosuppression in a subject, thereby increasing the risk of the subject developing cancer.

The disclosure also provides a method of treating a noncancerous condition in a subject at risk of developing a cancerous condition, comprising administering the antigen binding domain that bind CD20 of the disclosure to the subject to treat the noncancerous condition.

The disclosure also provides a method of treating a noncancerous condition in a subject at risk of developing a cancerous condition, comprising administering the protein comprising the antigen binding domain that bind CD20 of the disclosure to the subject to treat the noncancerous condition.

The disclosure also provides a method of treating a noncancerous condition in a subject at risk of developing a cancerous condition, comprising administering the multispecific protein comprising the antigen binding domain that bind CD20 of the disclosure to the subject to treat the noncancerous condition.

The disclosure also provides a method of treating a noncancerous condition in a subject at risk of developing a cancerous condition, comprising administering the immunoconjugate of the disclosure to the subject to treat the noncancerous condition.

The disclosure also provides a method of treating a noncancerous condition in a subject at risk of developing a cancerous condition, comprising administering the pharmaceutical composition of the disclosure to the subject to treat the noncancerous condition.

The disclosure also provides a method of treating a noncancerous condition in a subject at risk of developing a cancerous condition, comprising administering a CD20 binding protein of the disclosure to the subject to treat the noncancerous condition.

The disclosure also provides a method of preventing CD20 expressing cancer in a subject, comprising administering a therapeutically effective amount of the antigen biding domain that binds CD20 of the disclosure to the subject for a time sufficient to prevent the CD20 expressing cancer.

The disclosure also provides a method of preventing a CD20 expressing cancer in a subject, comprising administering a therapeutically effective amount of the protein comprising the antigen biding domain that binds CD20 of the disclosure to the subject for a time sufficient to prevent the CD20 expressing cancer.

The disclosure also provides a method of preventing a CD20 expressing cancer in a subject, comprising administering a therapeutically effective amount of the multispecific protein comprising the antigen biding domain that binds CD20 of the disclosure to the subject for a time sufficient to prevent the CD20 expressing cancer.

The disclosure also provides a method of preventing a CD20 expressing cancer in a subject, comprising administering a therapeutically effective amount of the CD20 antigen-binding protein comprising the antigen biding domain that binds CD20 of the disclosure to the subject for a time sufficient to prevent the CD20 expressing cancer.

The disclosure also provides a method of preventing a CD20 expressing cancer in a subject, comprising administering a therapeutically effective amount of the immunoconjugate comprising the antigen biding domain that binds CD20 of the disclosure to the subject for a time sufficient to prevent the CD20 expressing cancer.

The disclosure also provides a method of preventing a CD20 expressing cancer in a subject, comprising administering a therapeutically effective amount of the pharmaceutical composition comprising the antigen biding domain that binds CD20 of the disclosure to the subject for a time sufficient to prevent the CD20 expressing cancer.

The disclosure also provides a method of reducing the amount of CD20 expressing tumor cells in a subject, comprising administering the antigen biding domain that binds CD20 of the disclosure to the subject for a time sufficient to reduce the amount of CD20 expressing tumor cells.

The disclosure also provides a method of reducing the amount of CD20 expressing tumor cells in a subject, comprising administering the protein comprising the antigen biding domain that binds CD20 of the disclosure to the subject for a time sufficient to reduce the amount of CD20 expressing tumor cells.

The disclosure also provides a method of reducing the amount of CD20 expressing tumor cells in a subject, comprising administering the multispecific protein comprising the antigen biding domain that binds CD20 of the disclosure to the subject for a time sufficient to reduce the amount of CD20 expressing tumor cells.

The disclosure also provides a method of reducing the amount of CD20 expressing cells in a subject, comprising administering the immunoconjugate of the disclosure to the subject for a time sufficient to reduce the amount of CD20 expressing tumor cells.

The disclosure also provides a method of reducing the amount of CD20 expressing cells in a subject, comprising administering the pharmaceutical composition of the disclosure to the subject for a time sufficient to reduce the amount of CD20 expressing tumor cells.

The disclosure also provides a method of treating cancer in a subject, comprising administering a therapeutically effective amount of the multispecific protein comprising the antigen binding domain that binds CD20 to the subject for a time sufficient to treat the cancer, wherein the antigen binding domain that bind CD20 comprises
the VH of SEQ ID NO: 12 and the VL of SEQ ID NO: 14;
the VH of SEQ ID NO: 16 and the VL of SEQ ID NO: 18;
the VH of SEQ ID NO: 20 and the VL of SEQ ID NO: 22; or
the VH of SEQ ID NO: 24 and the VL of SEQ ID NO: 26.

In one aspect, the present disclosure provides methods of preventing cancer, the methods comprising administering a CD20-binding protein described herein to a subject in need thereof.

In one aspect, the present disclosure provides methods of treating a subject having cancer, the methods comprising administering a therapeutically effective amount of a lymphocyte expressing one or more of the CD20-binding proteins described to a subject in need thereof, whereby the lymphocyte induces or modulates killing of cancer cells in the subject.

In another aspect, the present disclosure provides methods of reducing tumor burden in a subject having cancer, the methods comprising administering a therapeutically effective amount of a lymphocyte expressing one or more of the CD20-binding proteins described herein to a subject in need thereof, whereby the lymphocyte induces killing of cancer cells in the subject. In another aspect, the present disclosure provides methods of increasing survival of a subject having cancer, the methods comprising administering a therapeutically effective amount of a lymphocyte expressing one or more of the CD20-binding proteins described to a subject in need thereof, whereby the survival of the subject is lengthened. Generally, the CD20-binding proteins can induce killing of cancer cells in the subject and result in reduction or eradication of the tumors/cancer cells in the subject.

In one aspect, the methods described herein are applicable to treatment of noncancerous conditions that are at risk of developing into a cancerous condition.

In one aspect, a method of targeted killing of a cancer cell is disclosed, the method comprising contacting the cancer cell with a CD20-binding protein described, whereby the CD20-binding protein induces killing of the cancer cell.

When a therapeutically effective amount is indicated, the precise amount of the CD20-binding proteins of the present disclosure to be administered can be determined by a physician with consideration of individual differences in age, weight, tumor size, extent of infection or metastasis, and condition of the subject.

Delivery systems useful in the context of the CD20-binding proteins of the invention may include time-released, delayed release, and sustained release delivery systems such that the delivery of the CD20-binding protein compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the invention.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation.

The administration of the CD20-binding proteins and compositions may be carried out in any manner, e.g., by parenteral or nonparenteral administration, including by aerosol inhalation, injection, infusions, ingestion, transfusion, implantation or transplantation. For example, the CD20-binding proteins and compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a subject by intradermal or subcutaneous injection. The compositions of CD20-binding proteins may be injected, for instance, directly into a tumor, lymph node, tissue, organ, or site of infection.

In one embodiment, administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

The following examples are provided to further describe some of the embodiments disclosed herein. The examples are intended to illustrate, not to limit, the disclosed embodiments.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Immunization Protocol and V Gene Recovery of CD79b Binders

The following protocol was used to prepare CD79b monoclonal antibodies (mAbs).

### AB239

A human immunoglobulin transgenic mouse strain (Ablexis®; AlivaMab, LLC.) was used to develop human CD79b monoclonal antibodies. The Ablexis® mice contained a chimeric human/mouse IgH locus (comprising of 32 human V alleles, 27 human D alleles and 6 human J alleles in natural configuration linked to the mouse C_{H} locus) together with fully human IgL locus (comprising of 18 V_{κ} alleles and 5 J_{κ} alleles and/or 29 V_{λ} alleles and 7 J_{λ} alleles linked to appropriate mouse Cλ or Cκ). Accordingly, the mice contained an inactivated endogenous Ig locus, and in response to immunization, the introduced human heavy and light chain transgenes undergo class switching and somatic mutation to generate high affinity human IgG monoclonal antibodies. The preparation and use of Ablexis®, and the genomic modifications carried by such mice, is described in U.S. Patent Pub. No. 2013/0167256.

When immunized with recombinant human CD79b (rhCD79b), this transgenic mouse produced human IgG antibodies specific to human CD79b.

For HYB650, the immunization strategy in Ablexis® kappa mice included repetitive immunizations multiple sites+intraperitoneal (RIMMS+IP) injections of rhCD79b (R&D Cas 9687-CD Lot: TLS021805A) in CL413 (InvivoGen cat #vac-c1413-5) (days 42, 49, and 56) or Sigma (Sigma, Catalog S6322) (days 72, 79, 86, and 114). On day 129, after sufficient titers were reached, mice were given a final boost of rhCD79b (R&D Cas 9687-CD Lot: TLS021805A)+CL413 (InvivoGen cat #vac-c1413-5)+CD40 (R&D cat #MAB440; lot: AHY181704A) 7 days prior to sorting. Spleens and mandibular, accessory mandibular, superficial parotid, proper axillary, accessory axillary, subiliac, sciatic, popliteal, gastric, pancreaticodoudenal, jejunal, and medial iliac lymph nodes were harvested and antigen-positive B cells were isolated by Fluorescence-activated cell sorting (FACS). Ten 384-well plates of sorted B cell supernatants were screened by cell-based MSD to identify mAbs with specific binding to human CD79b expressing SU-DHL-10 cells (CD79a/b expressing primary cell lines, AG000002270). Positive clones were sequenced, cloned and expressed in small scale.

### AB241

A human immunoglobulin transgenic mouse strain (Ablexis®; AlivaMab, LLC.) was used to develop human CD79b monoclonal antibodies. The Ablexis® mice contained a chimeric human/mouse IgH locus (comprising of 32 human V alleles, 27 human D alleles and 6 human J alleles in natural configuration linked to the mouse C_{H} locus) together with fully human IgL locus (comprising of 18 V_{κ} alleles and 5 J_{κ} alleles and/or 29 V_{λ} alleles and 7 J_{λ} alleles linked to appropriate mouse Cλ or Cκ). Accordingly, the mice contained an inactivated endogenous Ig locus, and in response to immunization, the introduced human heavy and light chain transgenes undergo class switching and somatic mutation to generate high affinity human IgG monoclonal antibodies. The preparation and use of Ablexis®, and the genomic modifications carried by such mice, is described in U.S. Patent Pub. No. 2013/0167256.

When immunized with recombinant human CD79b (rhCD79b), this transgenic mouse produced human IgG antibodies specific to human CD79b.

For HYB649, the immunization strategy in Ablexis® kappa mice consisted of RIMMS+IP injections rhCD79b (R&D Cas 9687-CD Lot: TLS021805A) in sigma adjuvant (Sigma, Catalog S6322) (days 0, 8, 13, and 20). On day 31, after sufficient titers were reached, mice were given a final boost of rhCD79b (R&D Cas 9687-CD Lot: TLS021805A)+anti-msCD40 (R&D cat #MAB440; lot: AHY181704A) 4 days prior to fusion. Spleens and mandibular, accessory mandibular, superficial parotid, proper axillary, accessory axillary, subiliac, sciatic, popliteal, gastric, pancreaticodoudenal, jejunal, and medial iliac lymph nodes were harvested and used to generate hybridomas. Sixty plates of hybridoma supernatants were screened by cell-based MSD to identify mAbs which exhibited binding to rhCD79b. After further confirmatory screenings, hybridoma supernatants from both screens that exhibited binding specific to human CD79b expressing SU-DHL-4 & SU-DHL-10 cells (CD79a/b expressing primary cell lines, AG000002269 & AG000002270, respectively) were sequenced, cloned and expressed in small scale.

### V Region Cloning

B-cells were lysed in RealTime Ready Cell Lysis Buffer (Roche) and the B cell lysate was used directly for cDNA synthesis using the Smarter cDNA synthesis kit (Clontech, Mount View, CA). To facilitate cDNA synthesis, oligodT was used to prime reverse transcription of all messenger RNAs followed by “5′ capping” with a Smarter IIA oligonucleotide. Subsequent amplification of the VH and VL fragments was performed using a 2-step PCR amplification using 5′ primers targeting the Smarter IIA cap and 3′ primers targeting consensus regions in CH1. Briefly, each 50 μl PCR reaction consists of 10 μM of forward and reverse primer mixes, 25 μl of PrimeStar Max DNA polymerase premix (Clontech), 2 μl of unpurified cDNA, and 21 μl of double-distilled H₂O. The cycling program starts at 94° C. for 3 min, followed by 35 cycles (94° C. for 10 Sec, 55° C. for 1 min, 68° C. for 1 min), and ends at 72° C. for 7 min. The second round PCR was performed with VL and VH 2nd round primers containing 15 bp complementary extensions that “overlap” respective regions in their respective Lonza mother vector (VH and VL). Second round PCR was performed with the following program: 94° C. for 3 min; 35 cycles (94° C. for 10 Sec, 65° C. for 1 min, 72° C. for 1 min), and ends at 72° C. for 7 min. In-Fusion® HD Cloning Kit (Clonetech, U.S.A.) was used for directional cloning of VL gene into Lonza huIgK or Lambda vector and VH gene into Lonza huIgG1 vector. To facilitate In-Fusion® HD Cloning, PCR products were treated with Cloning Enhancer before In-Fusion HD Cloning. Cloning and transformation were performed according to manufacturer's protocol (Clonetech, U.S.A.). Mini-prep DNAs were subjected to Sanger sequencing to confirm that complete V-gene fragments were obtained. The DNA plasmid DNA or glycerol stocks were sequenced at GENEWIZ using pre-designed primers to obtain v-region sequences. The resulting .abi files of V-region sequences were collected and analyzed by a Sanger V-region sequence analysis program. All V genes were cloned into the Lonza-based expression vector carrying the appropriate constant region of the desired human antibody isotype IgG1 AAS. A total of 147 antibodies were successfully cloned and proceeded for further characterization from Ablexis Mice.

### Example 2: Expression and Purification of Bispecific CD79b×CD3 and Trispecific CD79b×CD20×CD3 Antibodies

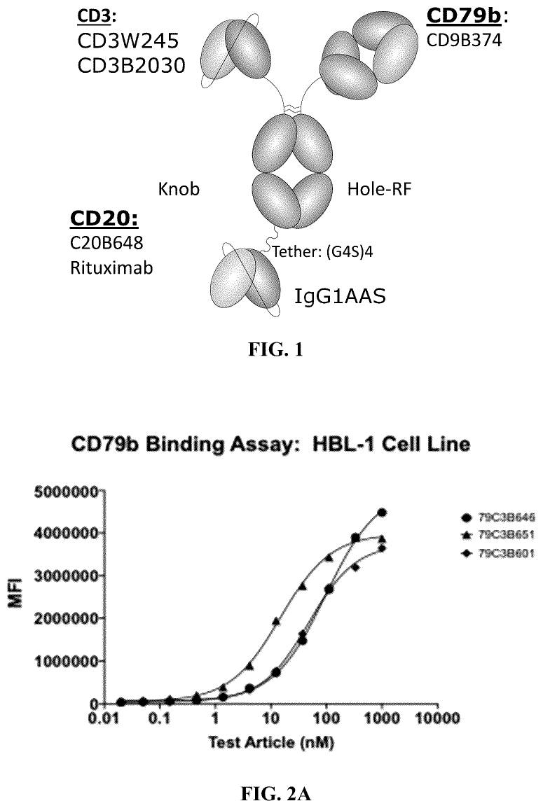

The CD79b×CD3 bispecific antibody (bsAb) is an immunoglobulin (Ig) G1 bispecific antibody that can bind simultaneously or individually to the cluster of differentiation (CD) 3 receptor complex on T lymphocytes and to CD79b on B lymphocytes. The CD79b×CD20×CD3 trispecific antibody is an immunoglobulin (Ig) G1 trispecific antibody that can bind simultaneously or individually to the CD3 receptor complex on T lymphocytes, and to the CD20 receptor complex on B lymphocytes and to the CD79b receptor complex on B lymphocytes. The antibody has mutations which reduce Fc binding to a Fey receptor and heterodimerization has been enhanced using the knobs-in-holes platform mutations. The trispecific antibody was developed to evaluate the therapeutic potential of dual targeting CD20 and CD79b for T cell redirection. An illustration of an exemplary CD79b×CD20×CD3 antibody is depicted in FIG. 1.

The antibodies were expressed in ExpiCHO-S™ cells (ThermoFisher Scientific; Waltham, MA, Cat #A29127) by transient transfection with purified plasmid DNA following the manufacturer's recommendations. Briefly, ExpiCHO-S™ cells were maintained in suspension in ExpiCHO™ expression medium (ThermoFisher Scientific, Cat #A29100) in an orbital shaking incubator set at 37° C., 8% CO₂ and 125 RPM. The cells were passaged and diluted prior to transfection to 6.0×10⁶ cells per ml, maintaining cell viability at 99.0% or better. Transient transfections were done using the ExpiFectamine™ CHO transfection kit (ThermoFisher Scientific, Cat #A29131). For each ml of diluted cells to be transfected, 0.5 microgram of bispecific encoding DNA (HC1:HC2:LC=1:2:2) and 0.5 microgram of pAdVAntage DNA (Promega, Cat #E1711) was used and diluted into OptiPRO™ SFM complexation medium. ExpiFectamine™ CHO reagent was used at a 1:4 ratio (v/v, DNA:reagent) and diluted into OptiPRO™. The diluted DNA and transfection reagent were combined for one minute, allowing DNA/lipid complex formation, and then added to the cells. After overnight incubation, ExpiCHO™ feed and ExpiFectamine™ CHO enhancers were added to the cells as per the manufacturer's Standard protocol. Cells were incubated with orbital shaking (125 rpm) at 37° C. for seven days prior to harvesting the culture broth. The culture supernatant from the transiently transfected ExpiCHO-S™ cells was clarified by centrifugation (30 min, 3000 ref) followed by filtration (0.2 μm PES membrane, Corning; Corning, NY).

The filtered cell culture supernatant was loaded onto a pre-equilibrated (1×DPBS, pH 7.2) MabSelect Sure Protein A column (GE Healthcare) using an AKTAXpress chromatography system. After loading, the column was washed with 10 column volumes of 1×DPBS, pH7.2. The protein was eluted with 10 column volumes of 0.1 M sodium (Na)-Acetate, pH 3.5. Protein fractions were neutralized immediately by the addition of 2.5 M Tris HCl, pH 7.2 to 20% (v/v) of the elution fraction volume. Peak fractions were pooled and loaded onto a CH1 column (Thermofisher). After loading, the column was washed with 10 column volumes of 1×DPBS, pH7.2. The protein was eluted with 10 column volumes of 0.1 M sodium (Na)-Acetate, pH 3.5. Protein fractions were partially neutralized by the addition of 2.5 M Tris HCl, pH 7.2 to 15% (v/v) of the final volume. The high molecular weight species were removed by preparative size exclusion chromatography (SEC) using Superdex 200 (GE Healthcare). Post sample injection, the column was developed with 1×DPBS and the major peak fractions were pooled, dialyzed into 10 mM Histidine, pH6.5 and filtered (0.2 μm).

The concentration of purified protein was determined by absorbance at 280 nm on a Dropsense spectrophotometer. The quality of the purified protein was assessed by cSDS and analytical size exclusion HPLC (Agilent HPLC system). The endotoxin level was measured using a turbidometric LAL assay (Pyrotell®-T, Associates of Cape Cod; Falmouth, MA).

### Example 4: Bispecific and Trispecific Antibodies Binding Characterization Binding of Bispecific CD79×CD3 Antibodies on CD79+Target Cells

The binding affinity of the CD79b binding arm of the CD79×CD3 bispecific molecules were assessed using cell lines that were validated by flow cytometry to have different endogenous expression levels of CD79b on the cell surface, shown in Table 4.

TABLE 4
CD79b Antigen Density of B Lymphoma Cell Lines

| | | CD79b Antigen |
|---|---|---|
| | | Density (Antigen |
| Cell Line | Cell Type | Number/cell) |
| HBL-1 | Diffuse large B-cell lymphoma line | 429,649 |
| OCI-LY-10 | Diffuse large B-cell lymphoma line | 38,885 |
| CARNAVAL | Diffuse large B-cell lymphoma line | 98,176 |
| WILL2 | Diffuse large B-cell lymphoma line | 3,824 |

Diffuse large B-cell lymphoma cell lines were incubated for 1 hour with CD79b×CD3 test molecules 79C3B646, 79C3B651, and 79C3B601 (1 uM starting concentration at 1:3 serial dilutions) at 37° C. All cells were washed with BD stain buffer (BD Biosciences; Cat #554657), centrifuged at 1200 RPM for 3 mins, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:200 dilution along with Aqua Fixable Live/Dead stain (Invitrogen; Cat #L34957) at a 1:400 dilution. All cells were washed with BD stain buffer (BD Biosciences; Cat #554657), centrifuged at 1200 RPM for 3 minutes, with supernatant discarded. Cells were analyzed using Intellicyt (Sartorius) flow cytometer and mean fluorescent intensity (MFI) was generated using Forcyt software (Sartorius). MFI was graphed and EC50 values generated using GraphPad PRISM v.8. Dose response curves were generated by transforming the x axis values using the formula x=lox. Data were then graphed using non-linear regression curve fit analysis “log(agonist) vs. response-variable slope (four parameter)”.

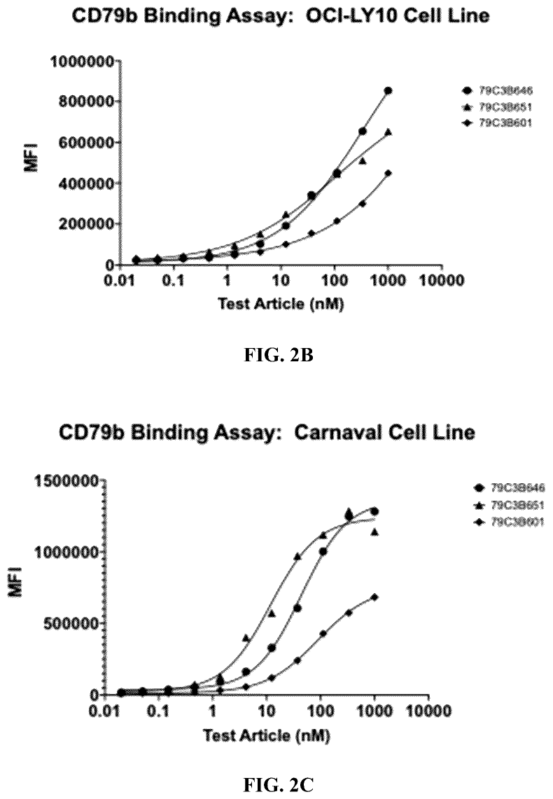

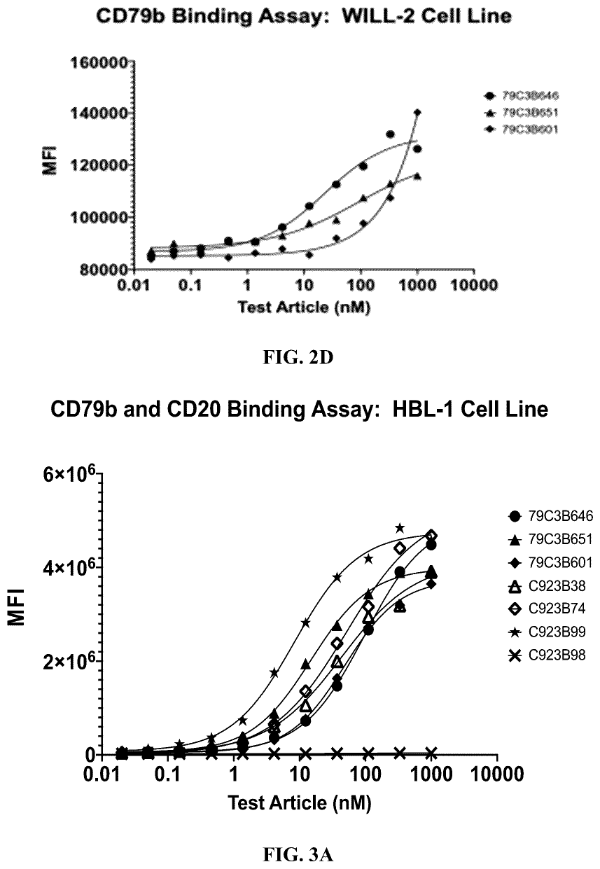

All CD79b×CD3 molecules showed good binding on cell lines expressing endogenous CD79b on the cell surface, with the CD79b binding arm of construct 79C3B651 showing the highest binding affinity across all tested cell lines, shown in FIGS. 2A-2D and Table 5.

TABLE 5
CD79b × CD3 Bispecifics Cell Binding EC50 Values

| | HBL-1 | OCI-LY10 | Carnaval | WILL-2 |
|---|---|---|---|---|
| | EC50 (nM) | EC50 (nM) | EC50 (nM) | EC50 (nM) |
| 79C3B646 | 97 | undetermined | 44 | undetermined |
| 79C3B651 | 15 | undetermined | 12 | undetermined |
| 79C3B601 | 48 | undetermined | 89 | undetermined |

### Binding of Trispecific CD79×CD20×CD3 Antibodies on CD79b+ and CD20 Target Cells

The binding affinity of the CD79b binding arm of the CD79×CD20×CD3 trispecific molecules as well as control CD79b×CD3 and Null×CD20×CD3 were assessed using cell lines that were validated by flow cytometry to have different endogenous expression levels of CD79b and CD20 on the cell surface, shown in Table 6.

TABLE 6
CD79b and CD20 Antigen Density of B Lymphoma Cell Lines

| | | CD79b Antigen | CD20 Antigen |
|---|---|---|---|
| | | Density | Density |
| | | (Antigen | (Antigen |
| Cell Line | Cell Type | Number/cell) | Number/cell) |
| HBL-1 | Diffuse large B-cell | 429,649 | 73,467 |
| | lymphoma line | | |
| OCI-LY-10 | Diffuse large B-cell | 38,885 | 67,352 |
| | lymphoma line | | |
| CARNAVAL | Diffuse large B-cell | 98,176 | 118,789 |
| | lymphoma line | | |
| WILL2 | Diffuse large B-cell | 3,824 | 314 |
| | lymphoma line | | |

Diffuse large B-cell lymphoma cell lines were incubated for 1 hour with CD79b×CD20×CD3 test molecules C923B74, C923B99, and C923B38; CD79×CD3 test molecules 79C3B646, 79C3B651, and 79C3B601 and Null×CD20×CD3 control molecule C923B98 (1 μM starting concentration at 1:3 serial dilutions) at 37° C. All cells were washed with BD stain buffer (BD Biosciences; Cat #554657), centrifuged at 1200 RPM for 3 minutes, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:200 dilution along with Aqua Fixable Live/Dead stain (Invitrogen; Cat #L34957) at a 1:400 dilution. All cells were washed with BD stain buffer (BD Biosciences; Cat #554657), centrifuged at 1200 RPM for 3 mins, with supernatant discarded. Cells were analyzed using Intellicyt (Sartorius) flow cytometer and mean fluorescent intensity (MFI) was generated using Forcyt software (Sartorius). MFI was graphed and EC50 values generated using GraphPad PRISM v.8. Dose response curves were generated by transforming the x axis values using the formula x=lox. Data was then graphed using non-linear regression curve fit analysis “log(agonist) vs. response-variable slope (four parameter)”.

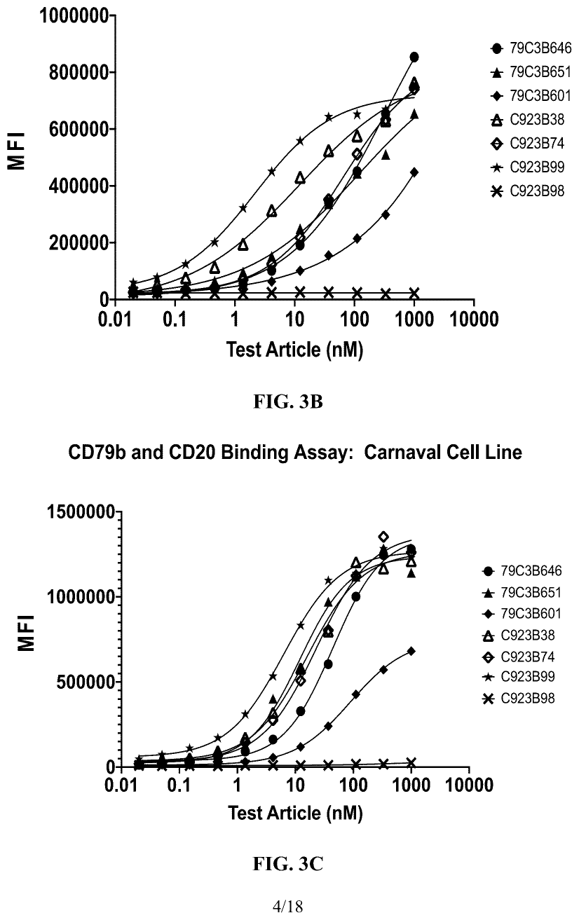

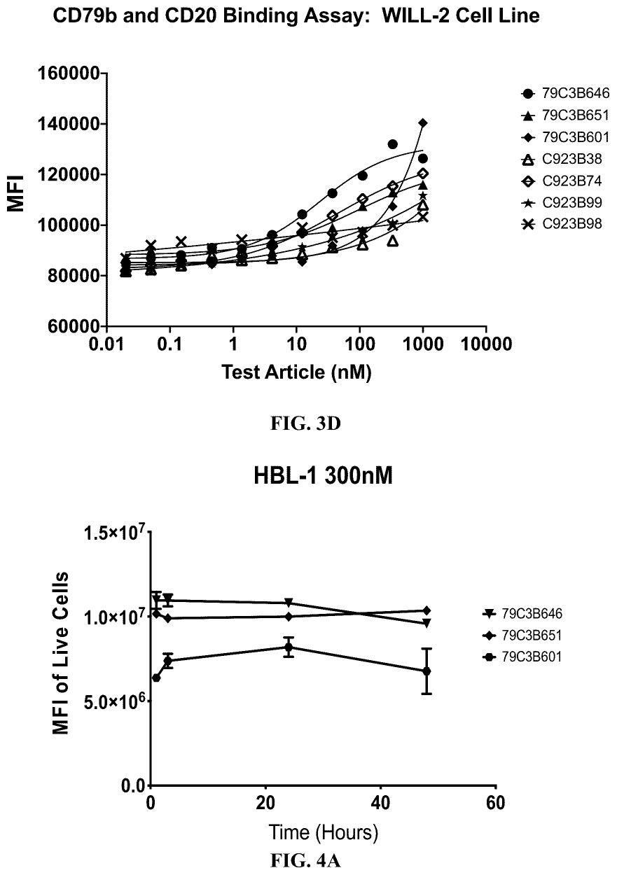

All CD79b×CD20×CD3 molecules showed good binding on cell lines expressing endogenous CD79b and CD20 on the cell surface, with some trispecific constructs showing better binding affinity across cell lines when compared to binding of CD79b×CD3 and CD20×CD3 control molecules, shown in FIGS. 3A-3D and Table 7. The CD79b binding arm of trispecific construct C923B99 showed the highest binding affinity across all tested cell lines, shown in FIGS. 3A-3D and Table 7.

TABLE 7
CD79bxCD20xCD3 Trispecific Cell Binding EC50 Values

| | HBL-1 | OCI-LY10 | Carnaval | WILL-2 |
|---|---|---|---|---|
| | EC50 | EC50 | EC50 | EC50 |
| | (nM) | (nM) | (nM) | (nM) |
| C923B38 | 43 | 12 | 16 | undetermined |
| C923B74 | 52 | 66 | 23 | undetermined |
| C923B99 | 8 | 2 | 6 | undetermined |
| 79C3B646 | 97 | undetermined | 44 | undetermined |
| 79C3B651 | 15 | undetermined | 12 | undetermined |
| 79C3B601 | 48 | undetermined | 89 | undetermined |
| C923B98 | undetermined | undetermined | Undetermined | undetermined |

### Kinetic Cell Binding of Bispecific CD79×CD3 Antibodies on CD79+Target Cells

The binding kinetics of the CD79b binding arm of the CD79×CD3 bispecific molecules were assessed over a time course using cell lines that were validated by flow cytometry to have different endogenous expression levels of CD79b on the cell surface, shown in Table 8.

TABLE 8
CD79b Antigen Density of B Lymphoma Cell Lines

| | | CD79b Antigen |
|---|---|---|
| | | Density (Antigen |
| Cell Line | Cell Type | Number/cell) |
| HBL-1 | Diffuse large B-cell lymphoma line | 429,649 |
| OCI-LY10 | Diffuse large B-cell lymphoma line | 38,885 |
| CARNAVAL | Diffuse large B-cell lymphoma line | 98,176 |

Diffuse large B-cell lymphoma cell lines were incubated for 1, 3, 24, and 48 hours with CD79b×CD3 test molecules 79C3B646, 79C3B651, and 79C3B601 (300 nM, 60 nM, 12 nM) at 37° C. At each time point, cells were washed with BD stain buffer (BD Biosciences; Cat #554657), centrifuged at 1200 RPM for 3 mins, with supernatant discarded. Cells were then stained for 30 minutes at 4° C. with BD stain buffer containing AlexaFluor 647 labeled anti-human IgG secondary antibody (Jackson Immuno; Cat #109-606-098) at a 1:200 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed and EC50 values generated using GraphPad PRISM v.8.

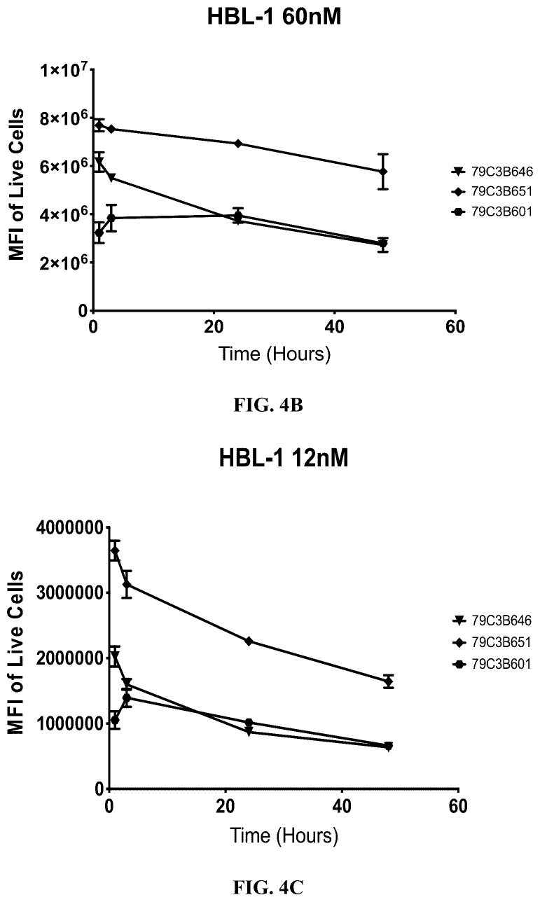

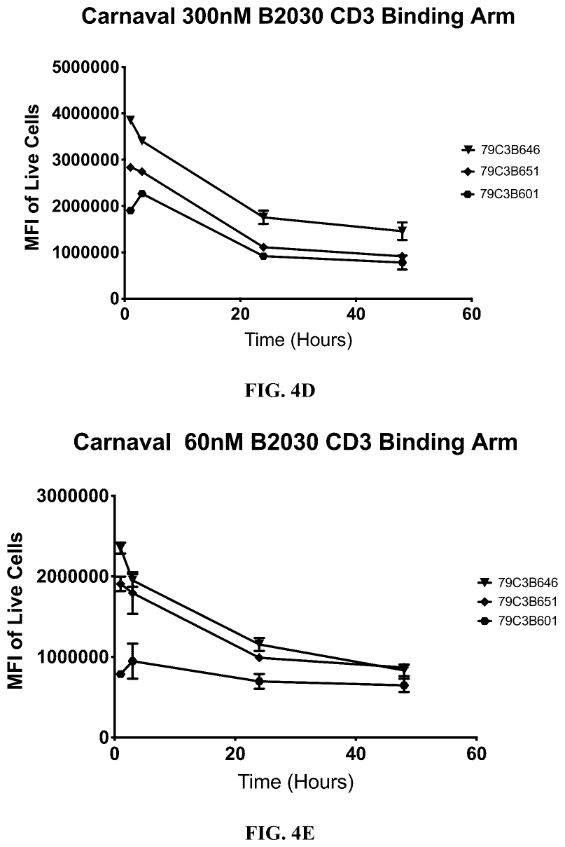

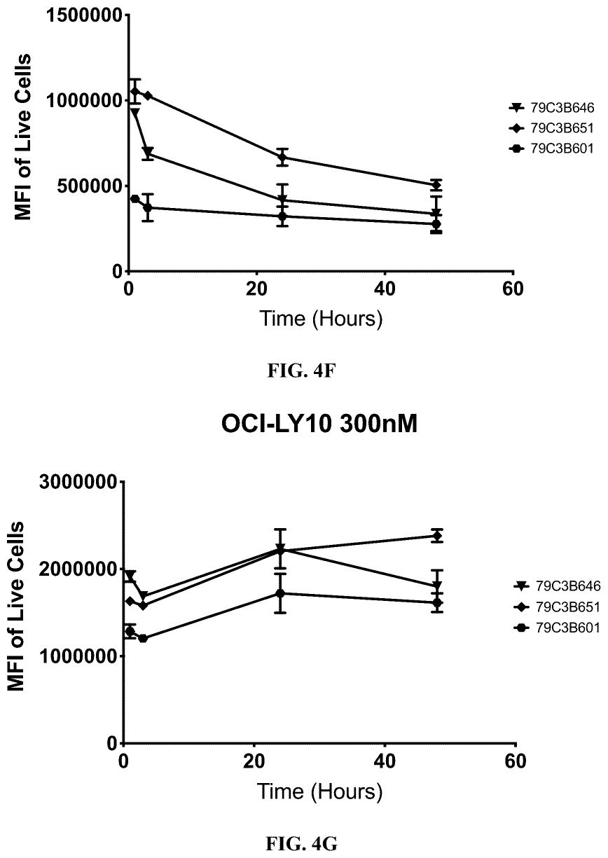

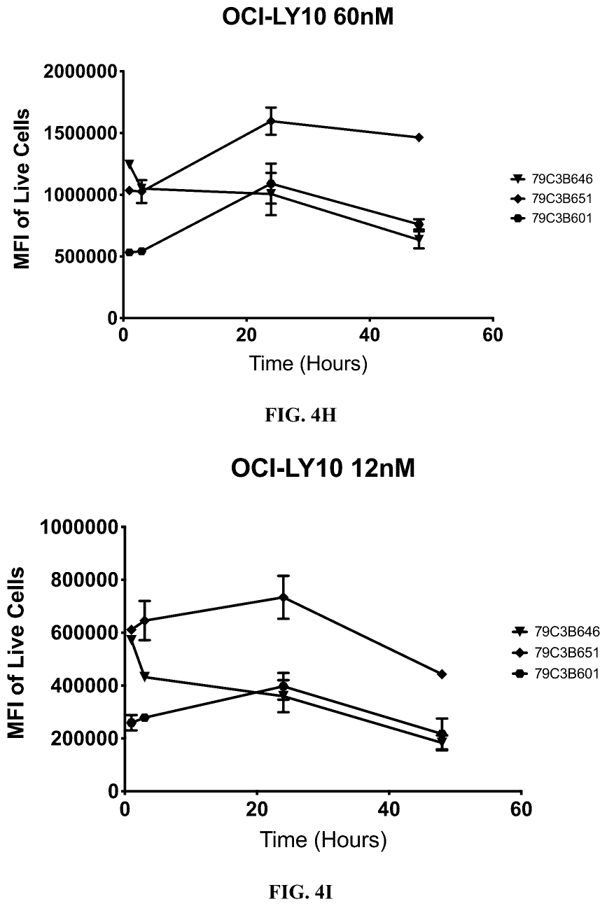

All CD79b×CD3 bispecific constructs showed steady CD79b binding kinetics with minimal loss of signal overtime, as shown in FIGS. 4A-4I. 79C3B651 showed superior binding kinetics and the least amount of signal loss over time, shown in FIGS. 4A-4I.

Kinetic Cell Binding of Trispecific CD79×CD20×CD3 Antibodies on CD79b⁺ and CD20⁺ Target Cells

The binding kinetics of the CD79b and CD20 binding arms of the CD79×CD20×CD3 trispecific molecules were assessed over a time course using cell lines that were validated by flow cytometry to have different endogenous expression levels of CD79b and CD20 on the cell surface, shown in Table 9.

TABLE 9
CD79b and CD20 Antigen Density of B Lymphoma Cell Lines

| | | CD79b Antigen | CD20 Antigen |
|---|---|---|---|
| | | Density | Density |
| | | (Antigen | (Antigen |
| Cell Line | Cell Type | Number/cell) | Number/cell) |
| HBL-1 | Diffuse large B-cell | 429,649 | 73,467 |
| | lymphoma line | | |
| OCI-LY-10 | Diffuse large B-cell | 38,885 | 67,352 |
| | lymphoma line | | |
| CARNAVAL | Diffuse large B-cell | 98,176 | 118,789 |
| | lymphoma line | | |

Diffuse large B-cell lymphoma cell lines were incubated for 1, 3, 24, and 48 hours with CD79b×CD20×CD3 test molecules C923B74, C923B99, and C923B38; CD79×CD3 test molecules 79C3B646, 79C3B651, and 79C3B601 and Null×CD20×CD3 control molecule C923B98 (300 nM, 60 nM, 12 nM) at 37° C. At each time point, cells were washed with BD stain buffer (BD Biosciences; Cat #554657), centrifuged at 1200 RPM for 3 mins, with supernatant discarded. Cells were then stained for 30 minutes at 4° C. with BD stain buffer containing AlexaFluor 647 labeled anti-human IgG secondary antibody (Jackson Immuno; Cat #109-606-098) at a 1:200 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed and EC50 values generated using GraphPad PRISM v.8.

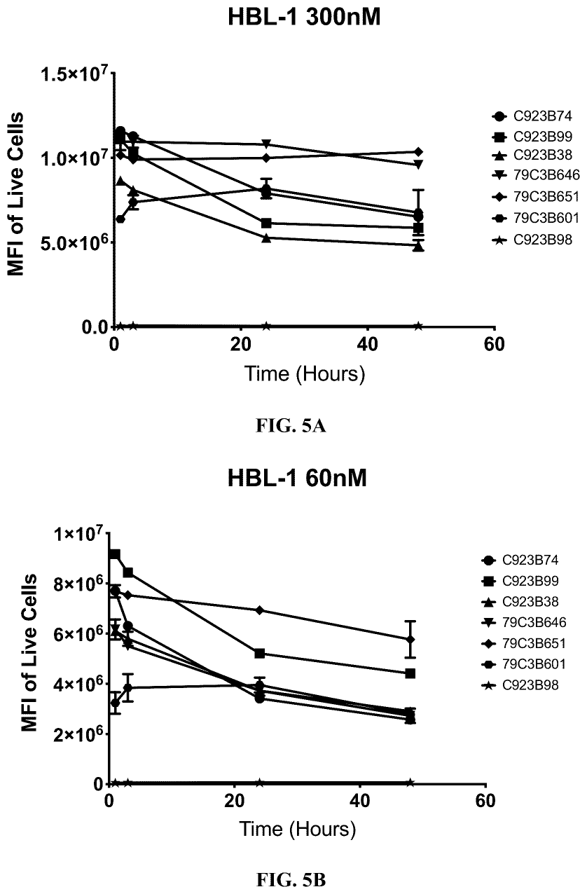

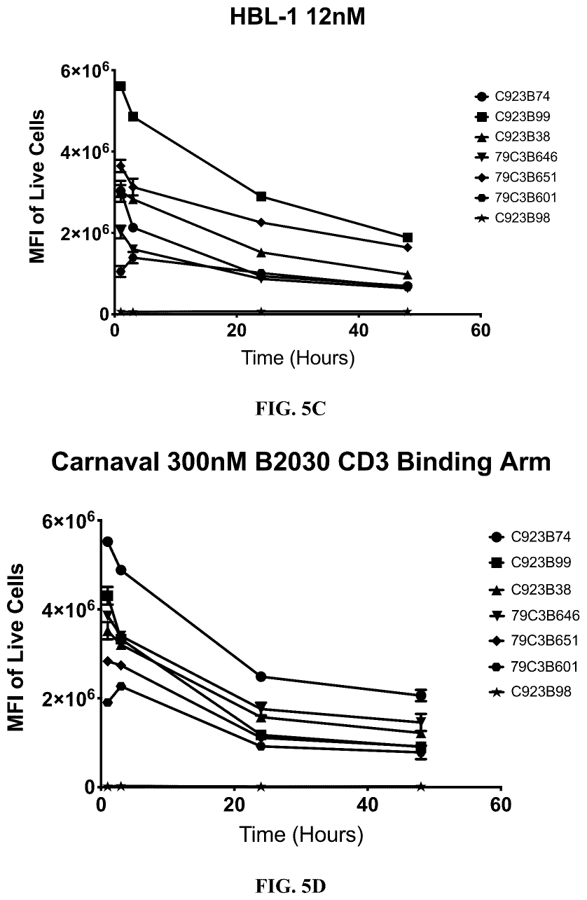

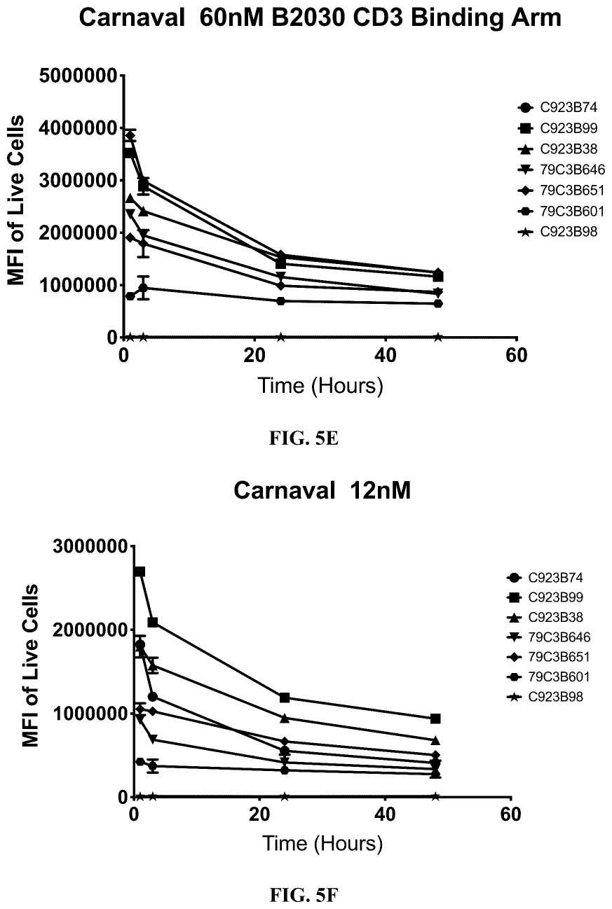

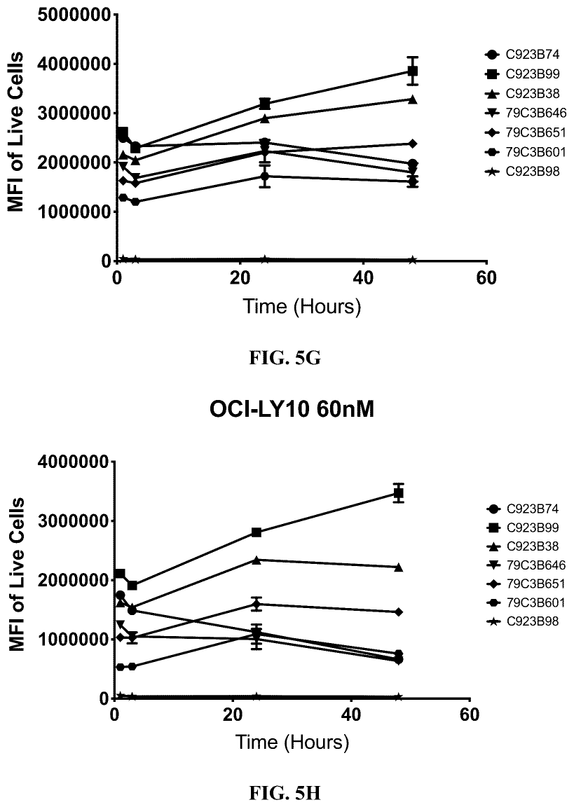

All CD79b×CD20×CD3 bispecific constructs showed steady CD79b binding kinetics with minimal loss of signal over time, shown in FIGS. 5A-5I. Trispecific construct C923B99 and bispecific construct 79C3B651, which both have the same CD79b and CD20 binding arms, showed superior binding kinetics and the least amount of signal loss over time, shown in FIGS. 5A-5I.

### Binding of Bispecific CD79×CD3 Antibodies and Trispecific CD79×CD20×CD3 Antibodies on Pan T-Cells

Binding of the CD3 arm of CD79×CD3 bispecific and CD79b×CD20×CD3 trispecific constructs was assessed using cryo-preserved, negatively selected, primary human CD3⁺ pan T cells. Primary human CD3⁺ pan T cells from four different donors were incubated for 1 hour with CD79b×CD20×CD3 test molecules C923B74, C923B99, and C923B38 or CD79×CD3 test molecules 79C3B646, 79C3B651 (1 uM starting concentration at 1:3 serial dilutions) at 37° C. All cells were washed with BD stain buffer (BD Biosciences; Cat #554657), centrifuged at 1200 RPM for 3 mins, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:300 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed using GraphPad PRISM v.8. Dose response curves were generated by transforming the x axis values using the formula x=lox. Data was then graphed using non-linear regression curve fit analysis “log(agonist) vs. response-variable slope (four parameter)”.

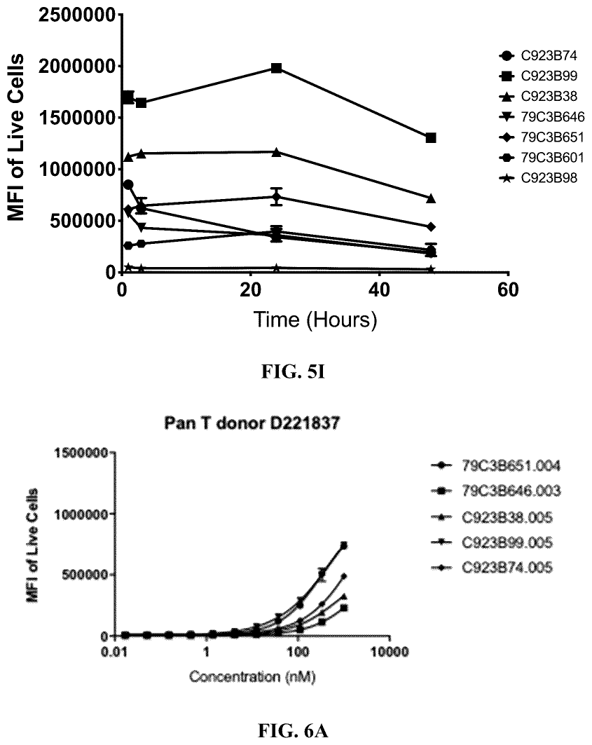

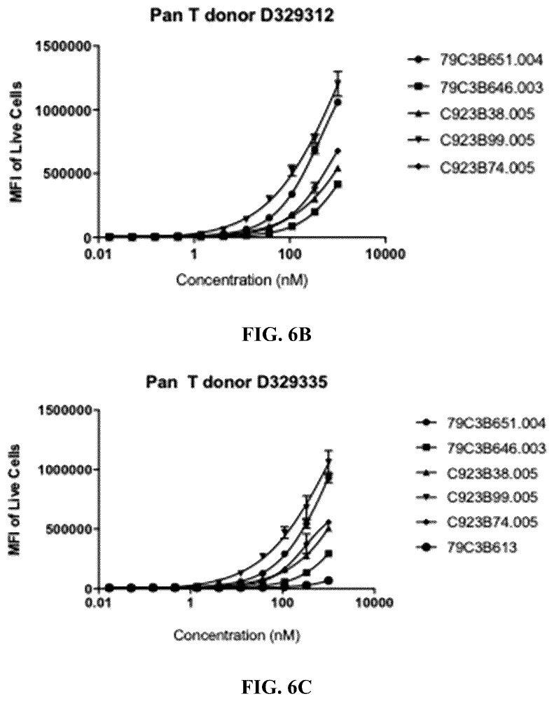

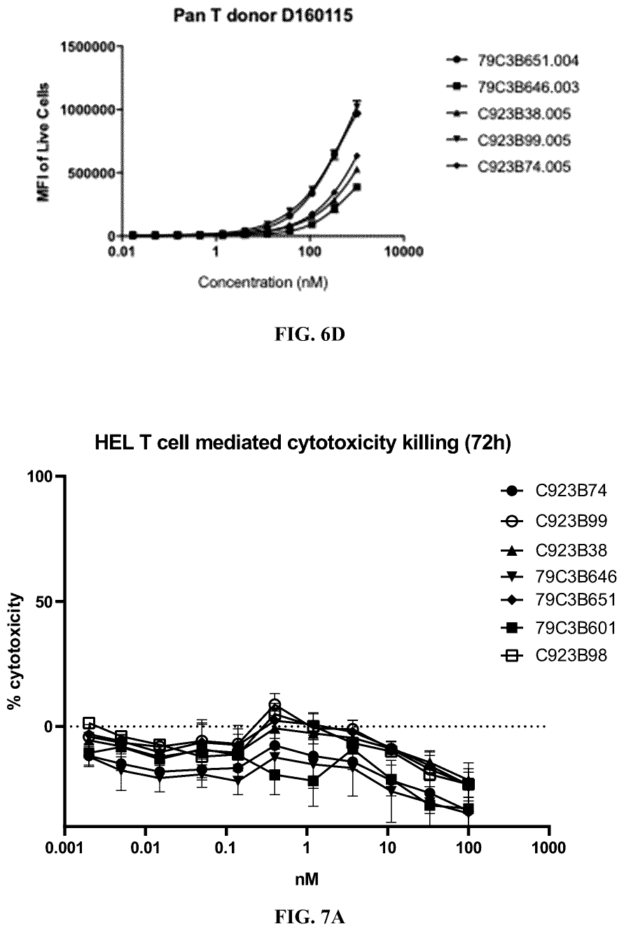

All CD79b×CD20×CD3 and CD79b×CD3 molecules showed moderate binding on all donor Pan T cells expressing endogenous CD3 on the cell surface, shown in FIGS. 6A-6D.

### Example 5: Functional Characterization: Antagonistic Activity of CD79×CD3 Bispecific and CD79×CD20×CD3 Trispecific Antibodies

Bispecific CD79×CD3 and Trispecific CD79×CD20×CD3 Mediated Cvtotoxicity Against CD79B⁺ and CD79B⁻ Target Cells

mKATE2 DLBCL target cells were maintained in complete RPMI (ThermoFisher, catalog #11875093) 1640 media containing 10% heat inactivated fetal bovine serum. Prior to the assay, antibodies were made at 3-fold serial dilutions in the at RPMI 1640 media containing 10% heat inactivated fetal bovine serum, at 4-fold expected final concentration. A volume of 50 μL of medium-diluted bsAb or trispecific Ab in each well of a 96-well plate were further diluted into 200 μL by adding a mix of target and effector cell suspension. The target cell lines were harvested by centrifuge at 400×g for 5 min, washed one time with phenol red-free RPMI 1640 media, counted and suspended in fresh complete phenol red-free RPMI 1640 media at 1×10⁶ cells/mL. Healthy donor T cells (isolated by CD3− negative selection provided by Discovery Life Sciences) were thawed in complete phenol red-free media (RPMI 1640 media containing 10% heat inactivated fetal bovine serum), counted and suspended in fresh complete phenol red-free RPMI 1640 media at 1×10⁶ cells/mL. Target cells and T cells were mixed to obtain 5:1 effector to target cell ratio. Cell suspension was added to antibody dilution wells according to plate layout (150 μL/well).

After mixing target and T cells with corresponding bsAb dilution, 80 μL from each well, containing 200 μl with 10000 target and 50000 T cells, were dispensed in a 384 well plate, in duplicate. Plates were sealed using a Breathe-Easy membrane seal. Next, co-cultures were placed in an IncuCyte ZOOM live-content imaging system, and images were automatically acquired in both phase and fluorescence channels every 6 hours for 3 to 6 days with a 4× objective lens (single whole well image). IncuCyte Zoom software was used to detect target cells based on mKATE2 expression using optimized process definition parameters. To measure the amount of target cells/well, the total red area was quantified, and raw values were exported in Excel (Microsoft Office). To quantify cancer cell killing over time, the average values for each replicate were pasted in Prism (GraphPad; version 7 for PC). Expansion indexes (EI) per timepoint were calculated by dividing value at Tx by T0. Growth inhibition (GI) was calculated by normalizing each timepoint to the value of the untreated well average at that timepoint. From the GI values, area under the curve (AUC) values were derived for each condition. After normalizing the AUC to the untreated control (target with effector), antibody concentrations were plotted against the AUC values as a dose response. EC50 values were generated using GraphPad PRISM v.8. Dose response curves were generated by transforming the x axis values using the formula x=lox. Data was then graphed using non-linear regression curve fit analysis “log(agonist) vs. response-variable slope (four parameter)”. Lead CD79b×CD3 bispecific antibodies and CD79b×CD20×CD3 trispecific antibodies (79C3B646, C923B74, 79C3B601, C923B38, 79C3B651, C923B99, 79C3B613, C923B98) were evaluated for cytotoxicity on HBL1 and OCI-Ly10 cells. IC50 (pM) values are listed in Table 10, Table 11, Table 12, and Table 13.

TABLE 10
HBL-1 killing Incucyte (Average of 2 independent experiments)

| Protein ID | CD79b | CD20 | CD3 | IC50 (pM) |
|---|---|---|---|---|
| 79C3B645 | CD9B330 | NA | CD3B2089 | 7189.0 |
| 79C3B646 | CD9B330 | NA | CD3B2030 | 257.4 |
| C923B73 | CD9B330 | C20B22 | CD3B2089 | 6805.0 |
| C923B74 | CD9B330 | C20B22 | CD3B2030 | 346.3 |
| 79C3B605 | CD9B374 | NA | CD3B2089 | 29549.0 |
| 79C3B601 | CD9B374 | NA | CD3B2030 | 203.9 |
| C923B36 | CD9B374 | C20B22 | CD3B2089 | 31040.0 |
| C923B38 | CD9B374 | C20B22 | CD3B2030 | 301.2 |
| 79C3B650 | CD9B643 | NA | CD3B2089 | 43314.0 |
| 79C3B651 | CD9B643 | NA | CD3B2030 | 32.5 |
| C923B95 | CD9B643 | C20B22 | CD3B2089 | 4891.0 |
| C923B99 | CD9B643 | C20B22 | CD3B2030 | 69.2 |

TABLE 11
OCI-Ly10 killing Incucyte (Average of 2 independent experiments)

| Protein ID | CD79b | CD20 | CD3 | IC50 (nM) |
|---|---|---|---|---|
| 79C3B645 | CD9B330 | NA | CD3B2089 | 18.0 |
| 79C3B646 | CD9B330 | NA | CD3B2030 | 18.3 |
| C923B73 | CD9B330 | C20B22 | CD3B2089 | 132.4 |
| C923B74 | CD9B330 | C20B22 | CD3B2030 | 25.6 |
| 79C3B605 | CD9B374 | NA | CD3B2089 | 54.3 |
| 79C3B601 | CD9B374 | NA | CD3B2030 | 11.7 |
| C923B36 | CD9B374 | C20B22 | CD3B2089 | 42.0 |
| C923B38 | CD9B374 | C20B22 | CD3B2030 | 8.0 |
| 79C3B650 | CD9B643 | NA | CD3B2089 | 7.0 |
| 79C3B651 | CD9B643 | NA | CD3B2030 | 4.7 |
| C923B95 | CD9B643 | C20B22 | CD3B2089 | 14.8 |
| C923B99 | CD9B643 | C20B22 | CD3B2030 | 5.6 |

TABLE 12
CARNAVAL killing (Incucyte)

| Protein ID | CD79b | CD20 | CD3 | IC50 (nM) |
|---|---|---|---|---|
| 79C3B646 | CD9B330 | NA | CD3B2030 | 1.393 |
| C923B74 | CD9B330 | C20B22 | CD3B2030 | 0.741 |
| 79C3B601 | CD9B374 | NA | CD3B2030 | 1.645 |
| C923B38 | CD9B374 | C20B22 | CD3B2030 | 0.465 |
| C923B99 | CD9B643 | C20B22 | CD3B2030 | 0.285 |

TABLE 13
Daudi killing (Incucyte)

| Protein ID | CD79b | CD20 | CD3 | IC50 (nM) |
|---|---|---|---|---|
| 79C3B646 | CD9B330 | NA | CD3B2030 | 0.597 |
| C923B74 | CD9B330 | C20B22 | CD3B2030 | 0.100 |
| 79C3B601 | CD9B374 | NA | CD3B2030 | 0.406 |
| C923B38 | CD9B374 | C20B22 | CD3B2030 | 0.071 |
| C923B99 | CD9B643 | C20B22 | CD3B2030 | <Conc tested |

### FACS T Cell Killing Data on Panel of Target Positive (CD79b+ and CD20+) and Target Negative (CD79B− and CD20−) Cell Lines

Functional activity of the CD79b×CD3 bispecific and CD79b×CD20×CD3 trispecific constructs was assessed at 72 hr time point in an in vitro T cell killing assay by flow cytometry using cell lines that were validated by flow cytometry to have different endogenous expression levels of CD79b and CD20 on the cell surface, shown in Table 17.

TABLE 14
CD79b and CD20 Antigen Density of B Lymphoma Cell Lines

| | | CD79b Antigen | CD20 Antigen |
|---|---|---|---|
| | | Density | Density |
| | | (Antigen | (Antigen |
| Cell Line | Cell Type | Number/cell) | Number/cell) |
| HBL-1 | Diffuse large B-cell | 429,649 | 73,467 |
| | lymphoma line | | |
| OCI-LY10 | Diffuse large B-cell | 38,885 | 67,352 |
| | lymphoma line | | |
| CARNAVAL | Diffuse large B-cell | 98,176 | 118,789 |
| | lymphoma line | | |
| K562 | Chronic myelogenous | 0 | 0 |
| | leukemia | | |
| HEL | Erythroleukemia | 0 | 0 |

Target cancer cells were maintained in complete RPMI 1640 (ThermoFisher, catalog #11875093) media containing 10% heat inactivated fetal bovine serum. Prior to the assay, antibodies were made at 3-fold serial dilutions in RPMI 1640 media containing 10% heat inactivated fetal bovine serum, at 4-fold expected final concentration. A volume of 50 μL of medium-diluted bispecific or trispecific Ab in each well of a 96-well plate were further diluted into 200 μL by adding a mix of target and effector cell suspension. The target cell lines were harvested by centrifuge at 400×g for 5 min, washed one time with RPMI 1640 media. Target cancer cells were stain targets with CellTrace CFSE (ThermoFisher; Cat #: C34554) diluted 1/5000. Healthy donor T cells (isolated by CD3− negative selection provided by Discovery Life Sciences) were thawed in complete media (RPMI 1640 media containing 10% heat inactivated fetal bovine serum), counted and suspended in fresh complete phenol red-free RPMI 1640 media at 1×10⁶ cells/mL. Target cells and T cells were mixed to obtain 5:1 effector to target cell ratio. Cell suspension was added to antibody dilution wells according to plate layout (150 μL/well). Cells were incubated for 72 hours with CD79b×CD3 or CD79b×CD20x×CD3 test molecules (100 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 15 minutes at room temperature with Fixable Live/Dead stain (ThermoFisher; Cat #65-0865-14) at a 1:1000 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 15), antibodies amount added as listed in the table. All cells were washed with BD stain buffer (BD Biosciences; Cat #554657), centrifuged at 1200 RPM for 3 mins, with supernatant discarded. Cells were analyzed using FACS Lyric (BD) flow cytometer and percent of cancer cell killing was generated using Cytobank. Percent of cancer cell killing was graphed and IC50 values generated using GraphPad PRISM v.8. Dose response curves were generated by transforming the x axis values using the formula x=lox. Data was then graphed using non-linear regression curve fit analysis “log(inhibitor) vs. response-variable slope (four parameter)”.

TABLE 15
Flow Panel Antibodies for T cell killing Assay

| Antibody | Conjugated | | Catalog | LOT | Amount added | |
|---|---|---|---|---|---|---|
| Name | Fluorophore | Vendor | Number | Number: | per well (μl) | |
| CD4 | V500 | BD Biosciences | 560768 | 9340575 | 2μ/well | |
| CD8 | PerCPCy5.5 | BD Biosciences | 560662 | 9290508 | 2 | μl/well |
| CD69 | PE | BD Biosciences | 560968 | 9049603 | 10 | μl/well |
| CD25 | BV421 | BD Biosciences | 562443 | 10302 | 2 | μl/well |

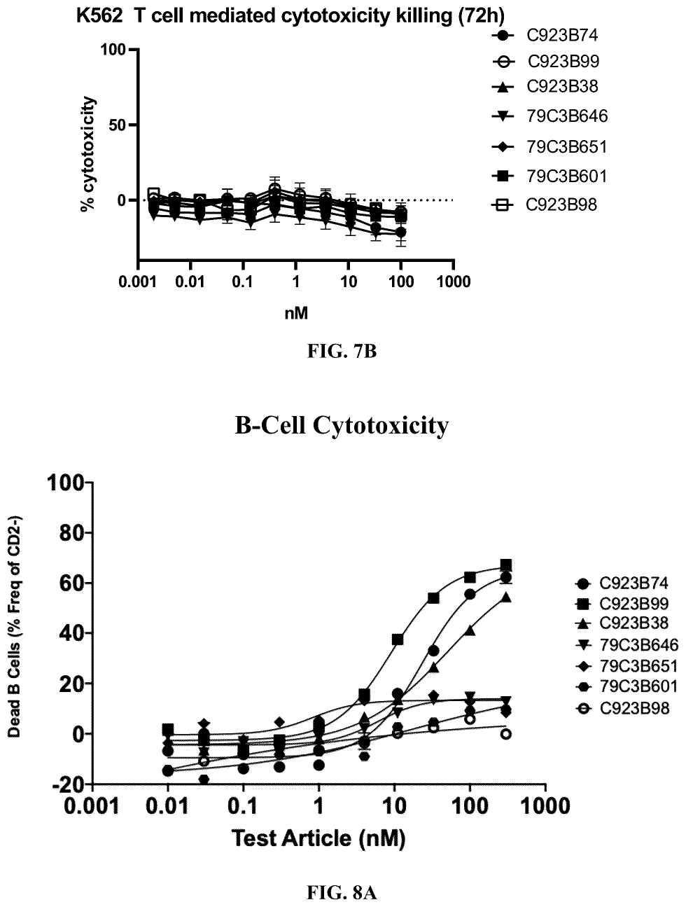

CD79b×CD20×CD3 trispecific mediated more potent cytotoxicity as compared to bispecific constructs in CD79b− and CD20− target positive cell lines. IC50 (pM) values are listed in Table 16. No killing has been observed in target negative cell lines (FIG. 7A-7B).

TABLE 16
Killing of target positive (CARNAVAL, OCI-Ly10) cell lines (FACS).

| | | | | CARNAVAL | HBL-1 | OCI-LY10 | |
|---|---|---|---|---|---|---|---|
| Protein ID | CD79b | CD20 | CD3 | IC50 (nM)* | IC50 (nM) | IC50 (nM)** | |
| 79C3B646 | CD9B330 | NA | CD3B2030 | 0.29 | 0.73 | >100 | nM |
| C923B74 | CD9B330 | C20B22 | CD3B2030 | 0.35 | 2.42 | 24.19 | |
| 79C3B601 | CD9B374 | NA | CD3B2030 | NA | 2.86 | >100 | nM |
| C923B38 | CD9B374 | C20B22 | CD3B2030 | 0.33 | 2.71 | 48.59 | |
| 79C3B651 | CD9B643 | NA | CD3B2030 | 0.25 | 2.20 | >100 | nM |

| | | | | | | |
|---|---|---|---|---|---|---|
| C923B99 | CD9B643 | C20B22 | CD3B2030 | 0.17 | 1.68 | 16.95 |

*average values of T cell mediated killing from 3 independent T cell donors
**average values of T cell mediated killing from 4 independent T cell donors

### Bispecific CD79b×CD3 Mediated Cytotoxicity Against Autologous B-Cells

Functional activity of the CD79b×CD3 bispecific constructs was assessed in an in vitro autologous B cell depletion assay. This functional assay utilizes PBMCs to focus on the killing of primary B cells as well as T cell activation on donor matched primary cells. Cryo-preserved PBMCs from 3 different human donors were incubated for 72 hours with CD79b×CD3 test molecules 79C3B646, 79C3B651, and 79C3B601 (300 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 10 minutes at room temperature with BD stain buffer containing Fc blocking agent (Accurate Chemical and Scientific Corp; Cat #NB309) and Near IR Fixable Live/Dead stain (Invitrogen; Cat #L10119) at a 1:400 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 17) at a 1:100 dilution. All cells were washed with BD stain buffer (BD Biosciences; Cat #554657), centrifuged at 1200 RPM for 3 mins, with supernatant discarded. Cells were analyzed using Intellicyt (Sartorius) flow cytometer and mean fluorescent intensity (MFI) was generated using Forcyt software (Sartorius). MFI was graphed and EC50 values generated using GraphPad PRISM v.8. Dose response curves were generated by transforming the x axis values using the formula x=lox. Data was then graphed using non-linear regression curve fit analysis “log(agonist) vs. response-variable slope (four parameter)”.

TABLE 17
Flow Panel Antibodies for Autologous B Cell Depletion Assay

| | Conjugated | | Catalog |
|---|---|---|---|
| Antibody Name | Fluorophore | Vendor | Number |
| Anti-human CD25 | BV650 | BD Biosciences | 563719 |
| Anti-Human CD4 | BV510 | Biolegend | 317444 |
| Anti-Human CD8 | PE-Cy7 | Biolegend | 301012 |
| Anti-Human CD20 | PE | Biolegend | 302306 |
| Anti-Human CD11c | AF647 | BD Biosciences | 565911 |
| Anti-Human CD2 | BV605 | BD Biosciences | 740391 |

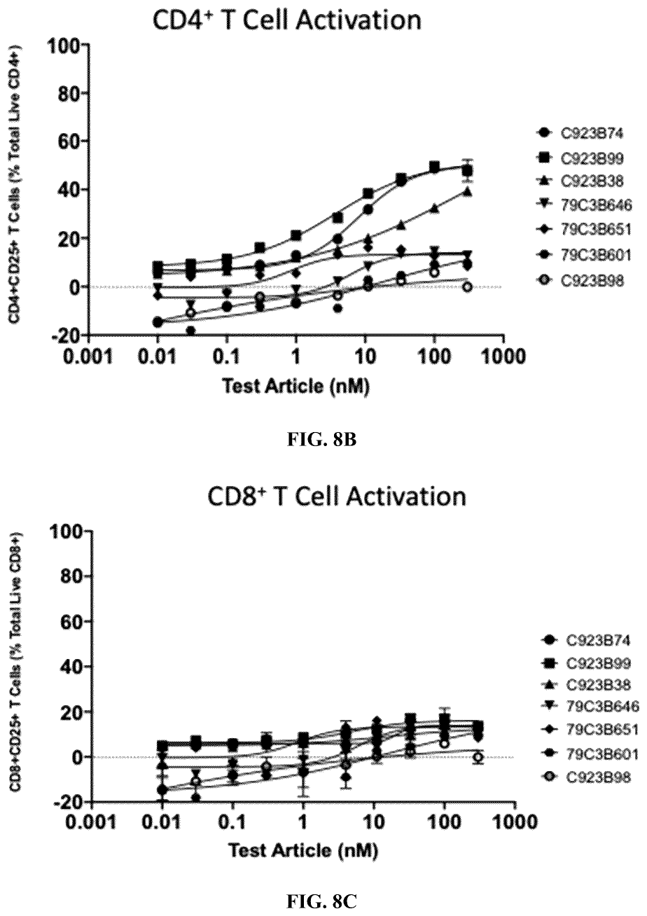

CD79b×CD3 bispecific constructs showed a maximum drug mediated cytotoxicity of 20 percent with low levels of CD4+ and CD8⁺ T cell activation as demonstrated by CD25 expression on these T cell subsets, as shown in FIGS. 8A-8C. The CD79b×CD20×CD3 trispecific has a synergistic effect on drug mediated cytotoxicity when compared to control molecules as shown in Table 18.

TABLE 40
CD79b × CD20 × CD3 EC50 Values and Maximum Cytotoxicity

| | | | | Donor 1 | | Donor 2 | | Donor 3 | |
|---|---|---|---|---|---|---|---|---|---|
| Construe | | | | EC50 | | EC50 | | EC50 | |
| Name | CD79b Arm | CD20 Arm | CD3 Arm | (nM) | Cₘₐₓ | (nM) | Cₘₐₓ | (nM) | Cₘₐₓ |
| C923 [symbol] | [symbol] -Fab | C [symbol] scFv | CD3 [symbol] | 78 | 34% | 23 | 66% | UD* | 40% |
| 79C [symbol] | | N/A | CD3 [symbol] | 40 | 9% | 6 | 14% | 8 | 35% |
| C [symbol] | N/A | C [symbol] scFv | CD3 [symbol] | UD* | 11% | UD* | 5% | 88 | 52% |
| C [symbol] | [symbol] -Fab | C [symbol] srFv | CD3 [symbol] | UD* | 35% | 9 | 67% | 23 | 66% |
| 79C3 [symbol] | | N/A | CD3 [symbol] | UD* | 5% | UD* | 13% | 17 | 34% |
| C [symbol] | N/A | C [symbol] scFv | CD3 [symbol] | UD* | 11% | UD* | 5% | 88 | 52% |
| C [symbol] | [symbol] -Fab | C [symbol] scFv | CD3 [symbol] | 1 | 24% | 50 | 30% | UD* | 50% |
| 79C3 [symbol] | | N/A | CD3 [symbol] | UD* | 10% | UD* | 10% | 20 | 32% |
| C23 [symbol] | N/A | C [symbol] scFv | CD3 [symbol] | UD* | 11% | UD* | 9% | 88 | 52% |

*Undetermined [symbol]
indicates data missing or illegible when filed

### Example 6: Biophysical Characterization

### Binding Affinity by SPR

General Protocol for SPR Affinity Assessment: Affinity assessment of the bispecific and trispecific constructs against human CD79b were measured using recombinantly expressed extracellular domain of CD79b short and long isoforms (CD9W7.001 and CD9W8.001, respectively) by Surface Plasmon Resonance (SPR) using a Biacore 8k SPR system (Biacore) at 25° C. in HBSP+ buffer. Cross-reactivity of the same antibody panel was also assessed against cyno and mouse antigens (CD9W1.001 and CD9W105.001, respectively). Briefly, a C1 sensor chip was immobilized with anti-human Fc (target immobilization levels of >400 RU) using vendor recommended amino coupling protocol. The test antibodies were captured through immobilized anti-Fc and was followed by the injection of different CD79b constructs at different concentration series (human CD79b short and long isoforms: 30 nM-0.37 nM at 3-fold dilutions; cyno and mouse CD79b: 3000 nM-37 nM at 3-fold dilutions). The association and dissociation phases were measured for 2 or 3 minutes and 30 minutes, respectively. Binding of the trispecifics (C923B168 and C923B3169) to CD3 was tested by injecting CD3W220.001 at 100 nM-1.23 nM at 3-fold dilutions, with association and dissociation phases were measured for 3 min and 15 min, respectively (CD79b-00478).

The raw binding sensorgrams were processed using Biacore Insight software (Biacore) by double-referencing and the processed sensorgrams were analyzed for cross-reactivity and filled to a 1:1 Langmuir model to obtain on-rates, off-rates and affinities.

SPR Binding Results: As shown in Table 19 and Table 20, She bispecific and trispecific antibodies bound to the human CD79b long isoform (hu CD79b long) with affinities from 0.02-0.06 nM, and to the CD79b short isoform (hu CD79b short) with affinities between 0.27-0.64 nM. The antibody panel showed very poor cross-reactivity to cyno CD79b (KD estimated>3000 nM) or did not bind to mouse CD79b. C923B168 binds recombinant CD3 antigen with an affinity of 0.5 nM. No quantitative kinetics/affinities were reported for those with complex kinetic binding profiles using the specified antigens, as noted in the summary tables below.

TABLE 19
Binding affinities for bispecific antibody constructs

| | KD to | KD to | |
|---|---|---|---|
| | hu CD79b | hu CD79b | KD to |
| Name | long (M) | short (M) | hu CD3 (M) |
| 79C3B601 | 4.6E−11 | 5.6E−10 | n.d** |
| 79C3B646 | 2.2E−11 | 5.8E−10 | n.d** |
| 79C3B651 | 5.2E−11 | 3.5E−10 | n.d** |
| 79C3B605 | n.d* | n.d* | n.d** |
| 79C3B645 | n.d* | n.d* | n.d** |
| 79C3B650 | n.d* | n.d* | n.d** |

*samples not submitted for SPR binding analysis
**Affinities for CD3 not determined due to complex SPR binding profiles observed for Cris 7b derived CD3 antibodies (historically observed results).

TABLE 20
Binding affinities for trispecific antibody constructs

| | KD to | KD to | KD to | KD to |
|---|---|---|---|---|
| | hu CD79b | hu CD79b | hu CD20 | hu CD3 |
| Name | long (M) | short (M) | (M) | (M) |
| C923B38 | 6.5E−11 | 6.4E−10 | n.d** | n.d** |
| C923B74 | 2.3E−11 | 3.9E−10 | n.d** | n.d** |
| C923B99 | 4.0E−11 | 2.7E−10 | n.d** | n.d** |
| C923B36 | n.d* | n.d* | n.d** | n.d** |
| C923B73 | n.d* | n.d* | n.d** | n.d** |
| C923B95 | n.d* | n.d* | n.d** | n.d** |
| C923B168 | 1.92E−10 | n.d* | n.d** | 4.96E−10 |
| C923B169 | 1.64E−10 | n.d* | n.d** | n.d** |

*samples not submitted for SPR binding analysis
**Affinities for CD20 or CD3 not determined due to SPR constraints with CD20 nanodiscs or complex binding profiles observed for Cris7b derived CD3 antibodies (historically observed results)

### Binding Epitope by HDX-MS

The CD79b epitopes bound by trispecific molecules CD9B3374 and CD9B3643 were mapped by Hydrogen Deuterium Exchange Mass Spectrometry (HDX-MS) according to the following protocol.

General Procedure for HDX-MS Data Acquisition. HDX-MS sample preparation was performed with automated HDx system (LEAP Technologies, Morrisville, NC). The columns and pump were: protease, protease type XIII (protease from Aspergillus saitoi, type XIII)/pepsin column (w/w, 1:1; 2.1×30 mm) (NovaBioAssays Inc., Woburn, MA); trap, ACQUITY UPLC BEH C18 VanGuard Pre-column (2.1×5 mm) (Waters, Milford, MA), analytical, Accucore C18 (2.1×100 mm) (Thermo Fisher Scientific, Waltham, MA); and LC pump, VH-P10-A (Thermo Fisher Scientific). The loading pump (from the protease column to the trap column) was set at 600 L/min with 0.10% aqueous formic acid. The gradient pump (from the trap column to the analytical column) was set from 9% to 35% acetonitrile in 0.1% aqueous formic acid in 20 min at 100 L/min.

MS Data Acquisition. Mass spectrometric analyses were carried out using an LTQ™ Orbitrap Fusion Lumos mass spectrometer (Thermo Fisher Scientific) with the capillary temperature at 275° C., resolution 120,000, and mass range (m/z) 300-1,800.

HDX-MS Data Extraction. BioPharma Finder 3.0 (Thermo Fisher Scientific) was used for the peptide identification of non-deuterated samples prior to the HDX experiments. HDExaminer version 2.5 (Sierra Analytics, Modesto, CA) was used to extract centroid values from the MS raw data files for the HDX experiments.

HDX-MS Data Analysis. The extracted HDX-MS data were further analyzed in Excel. All exchange time points (at pH 6.4 or pH 7.4 at 3.2° C.) were converted to the equivalent time points at pH 7.4 and 23° C.

### Results

HDX-MS analysis of CD9B374 and CD9B643 indicate binding to a nearly identical, conformational epitope of CD79 made up of residues 30-42 (SEDRYRNPKGSAC; SEQ ID NO: 66), 50-52 (PRF), 81-86 (EMENP; SEQ ID NO: 67), and 144-148 (GFSTL; SEQ ID NO: 68). The residue numbers are those of CD79B_Human (P40259).

### Thermal Stability of Trispecific CD79b×CD20×CD3 Antibodies by DSC and DSF

The thermal stability of C923B168 and C923B169 was determined by Differential Scanning Calorimetry (DSC) and differential scanning fluorimetry (DSF).

In this characterization, Tonset and Tagg were determined by DSF and the other thermal stability transitions of Tins were determined by DSC. As shown in Table 21, C923B168 and C923B169 have good thermal stability with Tonset>61° C. and Tm1>65° C.

TABLE 21
Transition temperatures for trispecific CD79bxCD20xCD3 antibodies:

| | Tonset | | Tm1 | | Tm2 | | Tm3 | | Tagg | |
|---|---|---|---|---|---|---|---|---|---|---|
| Sample ID | ° C. | σ | ° C. | σ | 0° C. | σ | ° C. | σ | 0° C. | σ |
| C923B168.008 | 61.3 | 0.15 | 65.5 | 0.03 | 73.5 | 0.18 | 77.3 | 0.07 | 73.8 | 0.37 |
| C923B169.008 | 61.7 | 0.07 | 68.4 | 0.03 | 75.1 | 0.44 | 77.7 | 0.21 | 74.2 | 0.5 |

### Example 7: Functional Characterization of CD79×CD20×CD3 Trispecific Antibodies Binding of Trispecific CD79b×CD20×CD3 Antibodies to Pan T-Cells

Binding of the CD3 arm of CD79b×CD20×CD3 trispecific constructs was assessed using cryo-preserved, negatively selected, primary human CD3⁺ pan T cells. Primary human CD3⁺ pan T cells from three different donors were incubated for 1 hour with CD79b×CD20×CD3 test molecules C923B169 and C923B168 (1 uM starting concentration at 1:3 serial dilutions) at 37° C. All cells were washed with BD stain buffer (BD Biosciences; Cat #554657), centrifuged at 1200 RPM for 3 mins, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:300 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed using GraphPad PRISM v.8. Dose response curves were generated by transforming the x axis values using the formula x=lox. Data was then graphed using non-linear regression curve fit analysis “log(agonist) vs. response-variable slope (four parameter)”.

All CD79b×CD20×CD3 molecules showed binding on all donor Pan T cells expressing endogenous CD3 on the cell surface, shown in Table 22.

TABLE 22
C923B169 and C923B168 CD79bxCD20xCD3
binding to Pan CD3 T cells.

| | Pan T cell binding, | | | Pan T cell max binding, | | |
|---|---|---|---|---|---|---|
| Construct | EC50 (nM) | | | MFI (×10⁶) | | |
| Name | Donor 1 | Donor 2 | Donor 3 | Donor 1 | Donor 2 | Donor 3 |
| C923B168 | 125 | 91 | 97 | 1.3 | 1.0 | 1.0 |
| C923B169 | UD* | UD* | UD* | 0.1 | 0.03 | 0.04 |

UD* = undetermined

FACS T Cell Killing Data on Panel of Target Positive (CD79b⁺ and CD20⁺) Cell Lines

Functional activity of the CD79b×CD20×CD3 trispecific constructs was assessed at 48 and 72 hr time point in an in vitro T cell killing assay by flow cytometry using cell lines that were validated by flow cytometry to have different endogenous expression levels of CD79b and CD20 on the cell surface, shown in Table 23.

TABLE 23
CD79b and CD20 Antigen Density of B Lymphoma Cell Lines

| | | CD79b Antigen | CD20 Antigen |
|---|---|---|---|
| | | Density | Density |
| | | (Antigen | (Antigen |
| Cell Line | Cell Type | Number/cell) | Number/cell) |
| OCI-LY10 | Diffuse large B-cell | 38,885 | 67,352 |
| | lymphoma line | | |
| CARNAVAL | Diffuse large B-cell | 98,176 | 118,789 |
| | lymphoma line | | |
| JEKO-1 | Mantle cell lymphoma | 280,000 | 50,000 |

Target cancer cells were maintained in complete RPMI-1640 (ThermoFisher, catalog #11875093) media containing 10% heat inactivated fetal bovine serum. Prior to the assay, antibodies were made at 3-fold serial dilutions in RPMI 1640 media containing 10% heat inactivated fetal bovine serum, at 4-fold expected final concentration. A volume of 50 μL of medium-diluted bispecific or trispecific Ab in each well of a 96-well plate were further diluted into 200 μL by adding a mix of target and effector cell suspension. The target cell lines were harvested by centrifuge at 400×g for 5 min, washed one time with RPMI 1640 media. Target cancer cells were stain targets with CellTrace CFSE (ThermoFisher; Cat #: C34554) diluted 1/5000. Healthy donor T cells (isolated by CD3− negative selection provided by Discovery Life Sciences) were thawed in complete media (RPMI 1640 media containing 10% heat inactivated fetal bovine serum), counted and suspended in fresh complete phenol red-free RPMI 1640 media at 1×10⁶ cells/mL. Target cells and T cells were mixed to obtain 5:1 effector to target cell ratio. Cell suspension was added to antibody dilution wells according to plate layout (150 μL/well). Cells were incubated for 48 and 72 hours with CD79b×CD20x×CD3 test molecules C923B169 and C923B168 (100 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 15 minutes at room temperature with Fixable Live/Dead stain (ThermoFisher; Cat #65-0865-14) at a 1:1000 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 24), antibodies amount added as listed in the table. All cells were washed with BD stain buffer (BD Biosciences; Cat #554657), centrifuged at 1200 RPM for 3 mins, with supernatant discarded. Cells were analyzed using FACS Lyric (BD) flow cytometer and percent of cancer cell killing was generated using Cytobank. Percent of cancer cell killing was graphed and 1C50 values generated using GraphPad PRISM v.8. Dose response curves were generated by transforming the x axis values using the formula x=lox. Data was then graphed using non-linear regression curve fit analysis “log(inhibitor) vs. response-variable slope (four parameter)”.

TABLE 24
Flow Panel Antibodies for T cell killing Assay

| | | | | | Amount | |
|---|---|---|---|---|---|---|
| Antibody | Conjugated | | Catalog | LOT | added per | |
| Name | Fluorophore | Vendor | Number | Number: | well (μl) | |
| CD4 | V500 | BD Biosciences | 560768 | 9340575 | 2 | /well |
| CD8 | PerCPCy5.5 | BD Biosciences | 560662 | 9290508 | 2 | ul/well |
| CD69 | PE | BD Biosciences | 560968 | 9049603 | 10 | ul/well |
| CD25 | BV421 | BD Biosciences | 562443 | 10302 | 2 | ul/well |

CD79b×CD20×CD3 trispecific mediated potent cytotoxicity. IC50 (nM) values and Max killing values are listed in Table 25 and Table 26.

TABLE 25
C923B169 and C923B168 CD79bxCD20xCD3 killing of target positive (CARNAVAL, OCI-
Ly10, JEK0-1) cell lines (FACS) at 48 hours. IC50 (nM) and percent of maximal killing
are listed in the table. Average values from 2 independent T cell donors.

| | Killing | | Killing | | | | | | Killing OCI- | | | |
|---|---|---|---|---|---|---|---|---|---|---|---|---|
| | CARNAVAL 1:1 | | CARNAVAL | | Killing JEKO-1 | | Killing JEKO-1 | | Ly10 1:1 | | Killing OCI-Ly10 | |
| | 48 hr | | 5:1 48 hr | | 1:1 48 hr | | 5:1 48 hr | | 48 hr | | 5:1 48 hr | |
| Protein ID | IC50 | Max | IC50 | Max | IC50 | Max | IC50 | Max | IC50 | Max | IC50 | Max |
| C923B169 | 110.3 | 49.7% | 0.179 | 87.2% | 27.307 | 60.8% | 0.027 | 93.5% | >100 | 22.2% | >100 | 19.7% |
| C923B168 | 13.6 | 58.8% | 0.012 | 95.0% | 7.466 | 65.3% | 0.002 | 96.7% | >100 | 25.1% | >100 | 29.8% |

TABLE 26
C923B169 and C923B168 CD79bxCD20xCD3 killing of target positive (CARNAVAL, OCI-
Ly10, JEK0-1) cell lines (FACS) at 72 hours. IC50 (nM) and percent of maximal killing
are listed in the table. Average values from 2 independent T cell donors.

| | Killing | | Killing | | | | | | Killing OCI- | | | |
|---|---|---|---|---|---|---|---|---|---|---|---|---|
| | CARNAVAL; | | CARNAVAL; | | Killing JEKO-1; | | Killing JEKO-1; | | Ly10; | | Killing OCI-Ly10; | |
| | 1:1 72hr | | 5:1 72hr | | 1:1 72hr | | 5:1 72hr | | 1:1 72hr | | 5:1 72hr | |
| Protein ID | IC50 | Max | IC50 | Max | IC50 | Max | IC50 | Max | IC50 | Max | IC50 | Max |
| C923B169 | 50.15 | 66.8% | 0.026 | 98.3% | 0.087 | 82.2 | 0.003 | 99.4% | >100 | 43.5% | 24.58 | 70.0% |
| C923B168 | 15.53 | 75.9% | 0.003 | 99.3% | 0.010 | 85.8% | 0.001 | 99.6% | 0.81 | 61.0% | 0.51 | 90.7% |

### C923B169 and C923B168 CD79b×CD20×CD3 Mediated Cytotoxicity Against Autologous B-Cells

Functional activity of the C923B169 and C923B168 CD79b×CD20×CD3 constructs was assessed in an in vitro autologous B cell depletion assay. This functional assay utilizes PBMCs to focus on the killing of primary B cells as well as T cell activation on donor matched primary cells. Cryo-preserved PBMCs from 3 different human donors were incubated for 72 hours with CD79b×CD20×CD3 test molecules C923B169 and C923B168 (300 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 10 minutes at room temperature with BD stain buffer containing Fc blocking agent (Accurate Chemical and Scientific Corp; Cat #NB309) and Near IR Fixable Live/Dead stain (Invitrogen; Cat #L10119) at a 1:400 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 27) at a 1:100 dilution. All cells were washed with BD stain buffer (BD Biosciences; Cat #554657), centrifuged at 1200 RPM for 3 mins, with supernatant discarded. Cells were analyzed using Intellicyt (Sartorius) flow cytometer. EC50 values generated using GraphPad PRISM v.8. Dose response curves were generated by transforming the x axis values using the formula x=lox. Data was then graphed using non-linear regression curve fit analysis “log(agonist) vs. response-variable slope (four parameter)”.

TABLE 27
Flow Panel Antibodies for Autologous B Cell Depletion Assay

| | Conjugated | | Catalog |
|---|---|---|---|
| Antibody Name | Fluorophore | Vendor | Number |
| Anti-human CD25 | BV650 | BD Biosciences | 563719 |
| Anti-Human CD4 | BV510 | Biolegend | 317444 |
| Anti-Human CD8 | PE-Cy7 | Biolegend | 301012 |
| Anti-Human CD20 | PE | Biolegend | 302306 |
| Anti-Human CD11c | AF647 | BD Biosciences | 565911 |
| Anti-Human CD2 | BV605 | BD Biosciences | 740391 |

CD79b×CD20×CD3 C923B169 and C923B168 constructs showed a maximum drug mediated cytotoxicity of 69-95 percent (Table 28) with low levels of CD4+ and CD8⁺ T cell activation as demonstrated by CD25 expression on these T cell subsets.

TABLE 28
C923B169 and C923B168 CD79bxCD20xCD3 killing of B cell in
the primary autologous B cell depletion assay at 72 hours.
EC50 (nM) and percent of maximal killing are listed in the
table. Values from 3 independent T cell donors listed.

| | D329465 | | D198013 | | D221837 | | Average values | |
|---|---|---|---|---|---|---|---|---|
| | | Max | | Max | | Max | | Max |
| | EC50 | Kill | EC50 | Kill | EC50 | Kill | EC50 | Kill |
| Name | (nM) | (%) | (nM) | (%) | (nM) | (%) | (nM) | (%) |
| C923B168 | 0.1 | 69 | 0.02 | 92 | 0.1 | 95 | 0.07 | 84 |
| C923B169 | 2.0 | 69 | 1.70 | 92 | 6.30 | 81 | 2.80 | 80 |

Sequence Listing:

SEQ ID NO: 1 CD20 Ab VH-CDR1
GYTFTSYNMH
SEQ ID NO: 2 CD20 Ab VH-CDR2
AIYPGNGDTS
SEQ ID NO: 3 CD20 Ab VH-CDR3
STYYGGDWYFNV
SEQ ID NO: 4 CD20 Ab VL-CDR1
RASSSVSYIH
SEQ ID NO: 5 CD20 Ab VL-CDR2
ATSNLAS
SEQ ID NO: 6 CD20 Ab VL-CDR3
QQWTSNPPT
SEQ ID NO: 7 CD20 Ab VH-CDR1
GYTFSSYNMH
SEQ ID NO: 8 CD20 Ab VH-CDR2
AIYPGAGDTS
SEQ ID NO: 9 CD20 Ab VH-CDR3
SNYYGSSGWYFDV
SEQ ID NO: 10 CD20 Ab VL-CDR1
RASLSVSSMH
SEQ ID NO: 11 CD20 Ab VL-CDR3
QQWIFNPPT
SEQ ID NO: 12 CD20 Ab VH
QVQLVQSGAEVKKPGSSVKVSCKASGYTFTSYNMHWVRQAPGQGLEWMGAIYPGNGDT
SYAQKFQGRVTITADKSTSTAYMELSSLRSEDTAVYYCARSTYYGGDWYFNVWGQGTLV
TVSS
SEQ ID NO: 13 CD20 Ab VH
CAGGTTCAGCTGGTTCAGTCTGGTGCCGAAGTGAAGAAACCTGGCTCCTCCGTGAAAG
TGTCCTGCAAGGCTTCCGGCTACACTTTTACCAGCTACAACATGCACTGGGTCCGACA
GGCCCCTGGACAAGGATTGGAATGGATGGGCGCTATCTACCCCGGCAACGGCGATAC
CTCTTACGCCCAGAAATTCCAGGGCAGAGTGACCATCACCGCCGACAAGTCTACCTCC
ACCGCCTACATGGAACTGTCCAGCCTGAGATCTGAGGACACCGCCGTGTACTACTGCG
CCCGGTCTACCTATTATGGCGGCGACTGGTACTTCAACGTGTGGGGCCAGGGAACCCT
GGTCACAGTCTCTTCT
SEQ ID NO: 14 CD20 Ab VL
AIQLTQSPSSLSASVGDRVTITCRASSSVSYIHWFQQKPGKAPKLIYATSNLASGVPSRFSG
ASGSGTDFTLTISSLQPEDFATYYCQQWTSNPPTFGQGTKLEIK
SEQ ID NO: 15 CD20 Ab VL
GCCATTCAGCTGACCCAGTCTCCATCCTCTCTGTCCGCCTCTGTGGGCGACAGAGTGAC
AATTACCTGCCGGGCCTCCTCCTCCGTGTCCTACATCCATTGGTTCCAGCAGAAGCCCG
GCAAGGCCCCTAAGCCTCTGATCTACGCCACCTCCAATCTGGCCTCTGGCGTGCCCTCC
AGATTTTCCGGATCTGGCTCTGGAACCGACTTTACCCTGACAATCTCCAGCCTGCAGCC
TGAGGACTTCGCCACCTACTACTGTCAGCAGTGGACCAGCAATCCTCCTACCTTTGGCC
AGGGCACCAAGCTGGAAATCAAG
SEQ ID NO: 16 CD20 Ab VH
QVQLV1QSGAEVKKPGSSVKVSCKASGYTFSSYNMHWVRQAPGQGLEWMGAIYPGAGDT
SYAQKFQGRVTITADESTSTAYMELSSLRSEDTAVYYCARSNYYGSSGWYFDVWGKGTT
VTVSS
SEQ ID NO: 17 CD20 Ab VH
CAGGTTCAACTGGTTCAGTCTGGCGCCGAAGTGAAGAAACCTGGCTCCTCCGTGAAGG
TGTCCTGCAAGGCTTCCGGCTACACCTTCTCCAGCTACAACATGCACTGGGTCCGACA
GGCCCCTGGACAAGGATTGGAATGGATGGGCGCTATCTACCCTGGCGCTGGCGATACC
TCTTACGCCCAGAAATTCCAGGGCAGAGTGACCATCACCGCCGACGAGTCTACCTCCA
CCGCCTACATGGAACTGTCCAGCCTGAGATCTGAGGACACCGCCGTGTACTACTGCGC
CCGGTCTAATTACTACGGCTCCAGCGGCTGGTACTTCGACGTGTGGGGAAAGGGCACC
ACCGTGACAGTCTCTTCT
SEQ ID NO: 18 CD20 Ab VL
EIVLTQSPATLSLSPGERATLSCRASLSVSSMHWYQQKPGQAPRLLIYATSNLASGIPARFS
GSGSGTDFTLTISSLEPEDFAVYYCQQWIFNPPTFGGGTKVEIK
SEQ ID NO: 19 CD20 Ab VL
GAGATCGTGCTGACCCAGTCTCCAGCCACACTGTCACTGTCTCCAGGCGAGAGAGCTA
CCCTGTCCTGTAGAGCCTCTCTGTCCGTGTCCTCCATGCACTGGTATCAGCAGAAGCCT
GGACAGGCCCCTCGGCTGCTGATCTACGCTACCTCTAATCTGGCCAGCGGTATCCCCG
CCAGATTTTCTGGTTCTGGCTCTGGCACCGACTTTACCCTGACCATCTCCAGCCTGGAA
CCTGAGGACTTCGCCGTGTACTACTGCCAGCAGTGGATCTTCAACCCTCCTACCTTTGG
CGGAGGCACCAAGGTGGAAATCAAG
SEQ ID NO: 20 CD20 Ab VH
QAYLQQSGAELVRPGASVKMSCKASGYTFTSYNMHWVKQTPRQGLEWIGAIYPGNGDTS
YNQKFKGKATLTVDKSSSTAYMQLSSLTSEDSAVYFCARVYYGSNYWYFDVWGTGTTV
TVSS
SEQ ID NO: 21 CD20 Ab VH
CAAGCATATCTGCAACAGAGCGGAGCTGAGCTGGTTCGGCCTGGCGCCTCTGTAAAAA
TGAGTTGCAAGGCCAGTGGTTATACATTCACATCATATAATATGCACTGGGTAAAGCA
AACTCCCCGACAGGGGCTTGAATGGATTGGCGCAATCTATCCCGGCAATGGGGATACA
TCCTACAATCAGAAATTCAAGGGCAAGGCAACACTGACCGTTGACAAATCCTCATCAA
CAGCCTACATGCAGCTCAGTTCCCTCACTAGCGAAGATTCTGCTGTGTATTTCTGTGCA
AGGGTGTATTATGGTTCTAATTACTGGTATTTCGATGTTTGGGGAACCGGAACTACCGT
AACTGTTTCTAGC
SEQ ID NO: 22 CD20 Ab VL
QIVLSQSPAILSASPGEKVTMTCRASSSVSYMHWYQQKPGSSPQVWIYATSNLASGVPVRF
SGSGSGTSYSLTISRVEAEDTATYYCQQWIFNPPTFGSGTKLEIR
SEQ ID NO: 23 CD20 Ab VL
CAAATAGTCCTTTCACAGTCCCCAGCTATTCTTTCAGCCTCTCCCGGTGAAAAGGTTAC
AATGACCTGCCGGGCAAGCTCCAGTGTCTCATATATGCACTGGTACCAACAAAAACCT
GGCAGTAGTCCTCAGGTGTGGATCTACGCTACAAGCAATCTCGCTTCCGGGGTTCCCG
TGAGGTTTAGCGGAAGCGGGTCTGGAACTAGTTATTCCTTGACAATTAGTCGGGTTGA
AGCCGAGGACACCGCCACTTACTATTGCCAACAGTGGATATTCAATCCACCCACCTTC
GGTTCAGGTACCAAGCTCGAAATCCGT
SEQ ID NO: 24 CD20 Ab VH
QAYLQQSGAELVRPGASVKMSCKTSGYTFSSYNMHWVKQTPRQALEWIGAIYPGNGDTS
YNQKFKGKATLTVDKSSSTAYMQLSSLTSEDSAVYFCTRSNYYGSSGWYFDVWGTGTTV
TVSS
SEQ ID NO: 25 CD20 Ab VH
CAAGCCTATCTTCAACAATCTGGGGCTGAGCTTGTCCGGCCAGGAGCCTCCGTCAAAA
TGAGCTGCAAAACCTCAGGTTATACTTTTAGTAGCTATAACATGCATTGGGTAAAACA
AACCCCCCGACAAGCATTGGAGTGGATAGGGGCCATATACCCCGGCAATGGAGACAC
AAGTTACAACCAGAAGTTTAAAGGCAAAGCTACACTCACAGTTGACAAATCCTCAAGT
ACTGCTTATATGCAACTCTCCTCTCTCACTTCCGAAGACAGTGCCGTATATTTTTGCAC
TCGGTCCAATTACTATGGATCTAGTGGCTGGTACTTTGACGTTTGGGGCACTGGGACA
ACTGTTACAGTGTCCAGC
SEQ ID NO: 26 CD20 Ab VL
QIVLSQSPAILSASPGEKVTMTCRASLSVSSMHWYQQKPGSSPKPWIYATSNLASGVPARF
SGSGSGTSYSLTISRVEAEDAATYYCQQWIFNPPTFGGGTKLEIK
SEQ ID NO: 27 CD20 Ab VL
CAGATTGTCCTGAGCCAATCCCCAGCAATTCTGAGTGCTAGCCCTGGAGAGAAGGTAA
CAATGACTTGTCGGGCATCCCTTAGCGTCTCATCCATGCATTGGTATCAACAAAAGCC
AGGTTCATCTCCAAAACCCTGGATTTACGCTACATCTAACCTGGCATCTGGGGTGCCTG
CCAGATTTAGTGGATCTGGTTCCGGCACATCATATTCCCTTACAATCAGCCGAGTGGA
AGCCGAGGATGCTGCAACCTATTACTGTCAACAATGGATATTTAACCCTCCCACCTTTG
GGGGTGGGACTAAACTCGAAATCAAG
SEQ ID NO: 28 Linker 1
GGSEGKSSGSGSESKSTGGS
SEQ ID NO: 29 Linker 2
GGGSGGGS
SEQ ID NO: 30 Linker 3
GGGSGGGSGGGS
SEQ ID NO: 31 Linker 4
GGGSGGGSGGGSGGGS
SEQ ID NO: 32 Linker 5
GGGSGGGSGGGSGGGSGGGS
SEQ ID NO: 33 Linker 6
GGGGSGGGGSGGGGS
SEQ ID NO: 34 Linker 7
GGGGSGGGGSGGGGSGGGGS
SEQ ID NO: 35 Linker 8
GGGGSGGGGSGGGGSGGGGSGGGGS
SEQ ID NO: 36 Linker 9
GSTSGSGKPGSGEGSTKG
SEQ ID NO: 37 Linker 10
IRPRAIGGSKPRVA
SEQ ID NO: 38 Linker 11
GKGGSGKGGSGKGGS
SEQ ID NO: 39 Linker 12
GGKGSGGKGSGGKGS
SEQ ID NO: 40 Linker 13
GGGKSGGGKSGGGKS
SEQ ID NO: 41 Linker 14
GKGKSGKGKSGKGKS
SEQ ID NO: 42 Linker 15
GGGKSGGKGSGKGGS
SEQ ID NO: 43 Linker 16
GKPGSGKPGSGKPGS
SEQ ID NO: 44 Linker 17
GKPGSGKPGSGKPGSGKPGS
SEQ ID NO: 45 Linker 18
GKGKSGKGKSGKGKSGKGKS
SEQ ID NO: 46 Linker 19
STAGDTHLGGEDFD
SEQ ID NO: 47 Linker 20
GEGGSGEGGSGEGGS
SEQ ID NO: 48 Linker 21
GGEGSGGEGSGGEGS
SEQ ID NO: 49 Linker 22
GEGESGEGESGEGES
SEQ ID NO: 50 Linker 23
GGGESGGEGSGEGGS
SEQ ID NO: 51 Linker 24
GEGESGEGESGEGESGEGES
SEQ ID NO: 52 Linker 25
GSTSGSGKPGSGEGSTKG
SEQ ID NO: 53 Linker 26
PRGASKSGSASQTGSAPGS
SEQ ID NO: 54 Linker 27
GTAAAGAGAAGGAAAGAAG
SEQ ID NO: 55 Linker 28
GTSGSSGSGSGGSGSGGGG
SEQ ID NO: 56 Linker 29
GKPGSGKPGSGKPGSGKPGS
SEQ ID NO: 57 Linker 30
GSGS
SEQ ID NO: 58 Linker 31
APAPAPAPAP
SEQ ID NO: 59 Linker 32
APAPAPAPAPAPAPAPAPAP
SEQ ID NO: 60 Linker 33
AEAAAKEAAAKEAAAAKEAAAAKEAAAAKAAA
SEQ ID NO: 61 Linker 34
GGGGSGGGGS
SEQ ID NO: 62 IgG1
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 63 IgG4
ASTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPSCPAPEFLGGPSVFLF
PPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGK
SEQ ID NO: 64 CD20 Ab VH-CDR3
VYYGSNYWYFDV
SEQ ID NO: 65 CD20 Ab VL-CDR1
RASSSVSVYMH

### REFERENCES

1. Kochenderfer, J. N. et al. Chemotherapy-refractory diffuse large B-cell lymphoma and indolent B-cell malignancies can be effectively treated with autologous T cells expressing an anti-CD19 chimeric antigen receptor. J Clin Oncol 33, 540-549 (2015).
2. Schuster, S. J. et al. Chimeric Antigen Receptor T Cells in Refractory B-Cell Lymphomas. N Engl J Med 377, 2545-2554 (2017).
3. Shalabi, H. et al. Sequential loss of tumor surface antigens following chimeric antigen receptor T-cell therapies in diffuse large B-cell lymphoma. Haematologica 103, e215-e218 (2018).
4. Sotillo, E. et al. Convergence of Acquired Mutations and Alternative Splicing of CD19 Enables Resistance to CART-19 Immunotherapy. Cancer Discov 5, 1282-1295 (2015).
5. Packard, T. A. & Cambier, J. C. B lymphocyte antigen receptor signaling: initiation, amplification, and regulation. F1000Prime Rep 5, 40 (2013).
6. Puri, K. D., Di Paolo, J. A. & Gold, M. R. B-cell receptor signaling inhibitors for treatment of autoimmune inflammatory diseases and B-cell malignancies. Int Rev Immunol 32, 397-427 (2013).
7. Polson, A. G. et al. Antibody-drug conjugates targeted to CD79 for the treatment of non-Hodgkin lymphoma. Blood 110, 616-623 (2007).
8. Palanca-Wessels, M. C. A. et al. Safety and activity of the anti-CD20 antibody-drug conjugate polatuzumab vedotin in relapsed or refractory B-cell non-Hodgkin lymphoma and chronic lymphocytic leukaemia: a phase 1 study. The Lancet Oncology 16, 704-715 (2015).
9. Astsaturov, I. A. et al. Differential expression of B29 (CD20) and mb-1 (CD79a) proteins in acute lymphoblastic leukaemia. Leukemia 10, 769-773 (1996).

1. An isolated protein comprising an antigen binding domain that binds Cluster of Differentiation 20 protein (CD20), wherein the antigen binding domain that binds CD20 comprises
a) a heavy chain complementarity determining region (HCDR) 1, a HCDR2 and a HCDR3 of a heavy chain variable region (VH) of SEQ ID NO: 12 and a light chain complementarity determining region (LCDR) 1, a LCDR2 and a LCDR3 of a light chain variable region (VL) of SEQ ID NO: 14;
b) a heavy chain complementarity determining region (HCDR) 1, a HCDR2 and a HCDR3 of a heavy chain variable region (VH) of SEQ ID NO: 16 and a light chain complementarity determining region (LCDR) 1, a LCDR2 and a LCDR3 of a light chain variable region (VL) of SEQ ID NO: 18;
c) a heavy chain complementarity determining region (HCDR) 1, a HCDR2 and a HCDR3 of a heavy chain variable region (VH) of SEQ ID NO: 20 and a light chain complementarity determining region (LCDR) 2, a LCDR2 and a LCDR3 of a light chain variable region (VL) of SEQ ID NO: 22; or
d) a heavy chain complementarity determining region (HCDR) 1, a HCDR2 and a HCDR3 of a heavy chain variable region (VH) of SEQ ID NO: 24 and a light chain complementarity determining region (LCDR) 1, a LCDR2 and a LCDR3 of a light chain variable region (VL) of SEQ ID NO: 26.

2. The isolated protein of claim 1, comprising the HCDR1, the HCDR2, the HCDR3, the LCDR1, the LCDR2 and the LCDR3 of
a) SEQ ID NOs: 1, 2, 3, 4, 5 and 6, respectively;
b) SEQ ID NOs: 7, 8, 9, 10, 5 and 11, respectively;
c) SEQ ID NOs: 1, 2, 64, 65, 5 and 11, respectively; or
d) SEQ ID NOs: 7, 2, 9, 10, 5 and 11, respectively.

3-10. (canceled)

11. The isolated protein of claim 1, wherein the antigen binding domain that binds CD20 comprises:
e) the VH of SEQ ID NO: 12 and the VL of SEQ ID NO: 14;
f) the VH of SEQ ID NO: 16 and the VL of SEQ ID NO: 18;
g) the VH of SEQ ID NO: 20 and the VL of SEQ ID NO: 22; or
h) the VH of SEQ ID NO: 24 and the VL of SEQ ID NO: 26.

12-53. (canceled)

54. The isolated protein of claim 1, wherein the protein comprises at least one mutation in a CH3 domain of the Ig constant region.

55. The isolated protein of claim 54, wherein the at least one mutation in the CH3 domain of the Ig constant region is selected from the group consisting of T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V and T350V/T366L/K392L/T394W, wherein residue numbering is according to the EU index.

56-61. (canceled)

62. An isolated multispecific protein comprising a first antigen binding domain that binds CD20 and a second antigen binding domain that binds a lymphocyte antigen.

63-66. (canceled)

67. The isolated multispecific protein of claim 62, wherein the lymphocyte antigen is selected from CD3, CD3 epsilon (CD38), CD8, KI2L4, NKG2E, NKG2D, NKG2F, BTNL3, CD186, BTNL8, PD-1, CD195, or NKG2C GD-e.

68-75. (canceled)

76. The isolated multispecific protein of claim 62, wherein the first antigen binding domain that binds CD20 comprises the HCDR1, the HCDR2, the HCDR3, the LCDR1, the LCDR2 and the LCDR3 of
i) SEQ ID NOs: 1, 2, 3, 4, 5 and 6, respectively;
j) SEQ ID NOs: 7, 8, 9, 10, 5 and 11, respectively;
k) SEQ ID NOs: 1, 2, 64, 65, 5 and 11, respectively; or
l) SEQ ID NOs: 7, 2, 9, 10, 5 and 11, respectively.

77. The isolated multispecific protein of claim 62, wherein the first antigen binding domain that binds CD20 comprises
m) the VH of SEQ ID NO: 12 and the VL of SEQ ID NO: 14;
n) the VH of SEQ ID NO: 16 and the VL of SEQ ID NO: 18;
o) the VH of SEQ ID NO: 20 and the VL of SEQ ID NO: 22; or
p) the VH of SEQ ID NO: 24 and the VL of SEQ ID NO: 26.

78-80. (canceled)

81. The isolated multispecific protein of claim 67, wherein the lymphocyte antigen is CD3ε.

82-96. (canceled)

97. A pharmaceutical composition comprising the isolated protein of claim 1 and a pharmaceutically acceptable carrier.

98. A polynucleotide encoding the isolated protein of claim 1.

99. A vector comprising the polynucleotide of claim 98.

100. A host cell comprising the vector of claim 99.

101-107. (canceled)

108. A method of treating a CD20 expressing cancer in a subject, comprising administering a therapeutically effective amount of the isolated protein of claim 1, or the pharmaceutical composition of claim 97 to the subject.

109-116. (canceled)

117. The method of claim 108, wherein the isolated protein is a multispecific protein.
